(19) United States
(12) Patent Application Publication
(10) Pub. No.: US 2021/0177982 A1
(43) Pub. Date: Jun. 17, 2021

(54) MUCUS-PENETRATING PEPTIDES, DELIVERY VEHICLES AND METHODS OF THERAPY

(71) Applicant: DNALite Therapeutics, Inc., San Francisco, CA (US)

(72) Inventors: Mubhij Ahmad, San Francisco, CA (US); Timothy Paul Day, San Francisco, CA (US); Cassandra Callmann, San Francisco, CA (US); Xixi Zhu, San Francisco, CA (US); Ismail Hafez, San Francisco, CA (US)

(21) Appl. No.: 17/098,053

(22) Filed: Nov. 13, 2020

Related U.S. Application Data
(63) Continuation of application No. PCT/US19/32484, filed on May 15, 2019.
(60) Provisional application No. 62/671,709, filed on May 15, 2018.

(51) Int. Cl. A61K 47/64; A61K 47/69

(52) U.S. Cl. CPC A61K 47/64; A61K 47/6929

(57) ABSTRACT

Provided are compositions including delivery vehicles with at least one mucus-penetrating property and mucus-penetrating peptides. Also disclosed are such compositions including a cargo and methods of making and using the same.

### CROSS-REFERENCES TO RELATED APPLICATIONS

This application is a Continuation of International Patent Application No. PCT/US2019/032484, filed May 15, 2019, which claims the benefit of U.S. Provisional Application No. 62/671,709 filed on May 15, 2018, the contents of which are incorporated herein by reference in their entirety.

### STATEMENT AS TO RIGHTS TO INVENTIONS MADE UNDER FEDERALLY SPONSORED RESEARCH OR DEVELOPMENT

This invention was made with Government support under National Science Foundation (NSF) Grant No. 1846078. The government has certain rights in the invention.

### BACKGROUND

Despite advances in gene therapy over the last 50 years, there remain many diseases that are recalcitrant to conventional methods, particularly in cases where a target location for delivery of therapy includes a layer of mucus.

### INCORPORATION BY REFERENCE

All publications, patents, and patent applications herein are incorporated by reference in their entirety to the same extent as if each individual publication, patent, or patent application was specifically and individually indicated to be incorporated by reference. In the event of a conflict between a term herein and a term in an incorporated reference, the term herein controls.

### SUMMARY OF THE INVENTION

One embodiment provides a composition comprising a peptide, a cargo and a delivery vehicle, wherein the peptide is a mucus-penetrating peptide, the peptide is conjugated directly or indirectly to the delivery vehicle to form a peptide-delivery vehicle conjugate, the delivery vehicle comprises at least one mucus-penetrating feature and the delivery vehicle partially or fully encapsulates the cargo. In some embodiments, the peptide or a portion thereof is exposed on the surface of the peptide-delivery vehicle conjugate.

In some embodiments, the peptide is selected from the group consisting of SEQ ID Nos. 1-35. In some embodiments, the average hydropathy of the amino acids of the peptide as measured by a Hodges score is less than or equal to 10 at pH 7. In some embodiments, the peptide comprises from 3 to 100 amino acids; and wherein the total number of amino acids with a Hodges score greater than 10 comprises no more than about 40% of the total number of amino acids in the peptide; and wherein the peptide comprises less than 5 pairs of adjacent amino acids where each amino acid of the pair has a Hodges score greater than 10. In some embodiments, the net charge of the peptide is less than about +2. In some embodiments, if the peptide comprises one or more cysteines, the cysteine does not contain a free thiol. In some embodiments, the composition is comprised within a nanoparticle. In some embodiments, the peptide is conjugated directly to the nanoparticle. In some embodiments, the nanoparticle has a diameter of no more than 500 nm. In some embodiments, the nanoparticle has a diameter of no more than 200 nm. In some embodiments, the nanoparticle has a diameter of no more than 100 nm.

In some embodiments, the nanoparticle comprises a lipid structure. In some embodiments, the lipid is selected from a liposome, a liposomal polyplex, a lipid nanoparticle and a lipoplex. In some embodiments, the delivery vehicle mucus-penetrating feature comprises one or more features selected from the group consisting of a mucus-penetrating surface modification to the delivery vehicle, a zwitterionic feature of the delivery vehicle, and a mucus-penetrating lipid composition of the delivery vehicle. In some embodiments, the surface modification is polyethylene glycol. In some embodiments, the surface modification is selected from one or more of poly (2-alkyl-2-oxazoline), poly(2-ethyl-2-oxazoline), and poly(2-methyl-2-oxazaline), a salt thereof, a di block polymer and a tri block polymer thereof. In some embodiments, the mucus-penetrating peptide is conjugated directly to the surface modification. In some embodiments, the peptide is covalently conjugated to the surface modification.

In some embodiments, the mucus-penetrating peptide is conjugated directly to the delivery vehicle. In some embodiments, the mucus-penetrating peptide is conjugated directly to a lipid structure comprised by the delivery vehicle. In some embodiments, the cargo comprises a nucleic acid. In some embodiments, the nucleic acid encodes for a protein or a biologically active portion of a protein directed to treating a disease or condition. In some embodiments, the disease or condition is a disease or condition that affects the gastrointestinal tract. In some embodiments, the disease or condition is at least one of: congenital diarrhea disease, irritable bowel syndrome, chronic inflammatory bowel disease, microvillus inclusion syndrome, familial polyposis (FAP), attenuated FAP, colorectal cancer, or any combination thereof. In some embodiments, the cargo comprises a dye. In some embodiments, the cargo comprises a drug or a therapeutic molecule. In some embodiments, the cargo comprises a protein. In some embodiments, the cargo comprises a nanoparticle. In some embodiments, the cargo comprises a small chemical molecule. In some embodiments, the peptide is selected from the group consisting of SEQ ID Nos. 1, 4, 5, 6, 7, 14, 20, 21, 22 and 29.

One embodiment provides a method of making a mucus-penetrating conjugate, the method comprising:

(a) selecting a peptide with at least one cell-penetrating property and at least one mucus-penetrating property;
(b) selecting a delivery vehicle with at least one mucus-penetrating property; and
(c) conjugating, indirectly or directly, the peptide and the delivery vehicle.

In some embodiments, the peptide is selected from the group consisting of SEQ ID Nos. 1-35. In some embodiments, the average hydropathy of the amino acids of the peptide as measured by a Hodges score is less than or equal to 10 at pH 7. In some embodiments, the average hydropathy of the amino acids of the peptide is less than or equal to 0.5 at pH 7. In some embodiments, the average hydropathy of the amino acid of the peptide is less than or equal to 0.5 at pH 7, as measured by a Fauchere score. In some embodiments, the peptide comprises from 3 to 100 amino acids; and wherein the total number of amino acids with a Hodges score greater than 10 comprises no more than about 40% of the total number of amino acids in the peptide; and

wherein the peptide comprises less than 5 pairs of adjacent amino acids where each amino acid of the pair has a Hodges score greater than 10. In some embodiments, the net charge of the peptide is less than about +2. In some embodiments, if the peptide comprises one or more cysteines, the cysteine does not contain a free thiol. In some embodiments, the peptide or a portion thereof is exposed on the surface of the mucus-penetrating conjugate. In some embodiments, the conjugate is comprised within a nanoparticle. In some embodiments, the nanoparticle is a lipid-containing nanoparticle. In some embodiments, the lipid is selected from a liposome, a liposomal polyplex, and a lipoplex. In some embodiments, the delivery vehicle mucus-penetrating property comprises one or more features selected from the group consisting of a mucus-penetrating surface modification to the delivery vehicle, a zwitterionic feature of the delivery vehicle, and a mucus-penetrating lipid composition of the delivery vehicle. In some embodiments, the delivery vehicle comprises a mucus-penetrating surface modification. In some embodiments, the surface modification is polyethylene glycol. In some embodiments, the surface modification is selected from one or more of poly (2-alkyl-2-oxazoline), poly(2-ethyl-2-oxazoline), and poly(2-methyl-2-oxazaline), a salt thereof, a di block polymer and a tri block polymer thereof. In some embodiments, the delivery vehicle partially or fully encapsulates a cargo. In some embodiments, the cargo comprises a nucleic acid.

In some embodiments, the nucleic acid encodes for a protein or a biologically active portion of a protein directed to treating a disease or condition of the gastrointestinal tract. In some embodiments, the disease or condition is a disease or condition affecting the gastrointestinal tract. In some embodiments, the disease or condition is at least one of: congenital diarrhea disease, irritable bowel syndrome, chronic inflammatory bowel disease, microvillus inclusion syndrome, familial polyposis (FAP), attenuated FAP, colorectal cancer, or any combinations thereof. In some embodiments, the nucleic acid encodes for a protein or biologically active portion of a protein selected from adenomatous polyposis coli (APC), defensin (HD-5), Myo5B, IL-10 and defensin alpha 6 (HD-6). In some embodiments, the cargo comprises a dye. In some embodiments, the cargo comprises a drug or a therapeutic molecule. In some embodiments, the cargo comprises a protein. In some embodiments, the cargo comprises a nanoparticle. In some embodiments, the cargo comprises a small chemical molecule. In some embodiments, for step (a) the peptide is first selected from Table 1, and wherein the selected peptide is modified to comprise mucus-penetrating properties by altering one or more amino acids of the peptide such that the average hydropathy of the amino acids of the modified peptide as measured by a Hodges score is less than or equal to 10 at pH 7. In some embodiments, the total number of amino acids in the modified peptide with a Hodges score greater than 10 comprises no more than about 40% of the total number of amino acids in the modified peptide; and wherein the modified peptide comprises less than 5 pairs of adjacent amino acids where each amino acid of the pair has a Hodges score greater than 10. In some embodiments, the net charge of the modified peptide is less than about +2. In some embodiments, if the modified peptide comprises one of more cysteines, the cysteine does not contain a free thiol.

In some embodiments, the peptide is selected from the group consisting of SEQ ID Nos. 1, 4, 5, 6, 7, 14, 20, 21, 22 and 29

One embodiment provides a method of delivering a gene therapy comprising administering a composition according to this disclosure. One embodiment provides a method of treating a disease or condition characterized by having at least one tissue targeted for therapy wherein the tissue comprises a layer of mucus, the method comprising administering a composition according to this disclosure. In some embodiments, the tissue targeted for therapy is selected from one or more of the eye, the gastrointestinal tract, the colon, the small intestine, the lung, and the cervix. In some embodiments, the disease or condition is selected from familial polyposis (FAP), attenuated FAP, colorectal cancer, chronic inflammatory bowel disease, irritable bowel syndrome, congenital diarrhea disease, microvillus inclusion syndrome, and any combinations thereof.

### BRIEF SUMMARY

Disclosed herein are delivery vehicles for therapy comprising a cargo and having mucus-penetrating as well as cell-penetrating properties. Diseases of the epithelium, such as colon cancer, cystic fibrosis, Crohn's disease and lung cancer, contribute to a significant portion of morbidity and mortality every year. Delivery of therapeutics, such as nucleic acids, small molecules, biologics and large molecules to mucosal epithelial cells for therapeutic purposes is made challenging by the physical barrier of the mucus. Accordingly, provided herein are delivery vehicles to penetrate a mucus layer and carry a cargo to the target tissues and cells. The provided delivery vehicles herein include mucus-penetrating features such as mucus-penetrating delivery vehicle compositions and mucus-penetrating polymer coatings and they are further coupled with mucus-penetrating peptides (MPPs) to have increased transport ability through the mucus associated with the target tissues. The combination of the MPPs with the mucus-penetrating features of a delivery vehicle allows the cargo to be delivered into the cells, rather than release of the cargo outside of the cells, which is the case with most clinically proven current applications of mucus penetrating systems which provide for only release outside the cell.

Provided herein are compositions having both a peptide and a delivery vehicle. The peptides of the composition are cell-penetrating and mucus-penetrating (these peptides are referred to herein as MPPs). The delivery vehicle also includes at least one mucus-penetrating feature. The peptide of the composition is conjugated directly or indirectly to the delivery vehicle, and the peptide or a portion thereof is exposed on the surface of the peptide-delivery vehicle conjugate.

The delivery vehicle may be a nanoparticle. In some cases, a delivery vehicle can have a diameter of from about 10 nm to about 100 nm, from about 100 nm to about 200 nm, from about 200 nm to about 300 nm, from about 300 nm to about 400 nm, and from about 400 nm to about 500 nm as measured by dynamic light scattering. The nanoparticle delivery vehicle may have a diameter of no more than 500 nm, no more than about 200 nm or no more than about 100 nm. In some embodiments, a delivery vehicle can be from about 1 nm to about 150 nm in diameter. In some embodiments, the nanoparticle is a lipid-containing nanoparticle. In some cases, a delivery vehicle can include a lipid structure such as a lipid nanoparticle, a liposome, a liposomal polyplex, or a lipoplex.

The compositions provided herein include delivery vehicles, including nanoparticles, where the delivery vehicle itself has at least one mucus-penetrating feature. Such mucus-penetrating features include, for example, a zwitterionic feature of the delivery vehicle or a lipid composition that confers mucus-penetrating properties to the delivery vehicle. A zwitterionic feature may include the formation of a delivery vehicle such as a nanoparticle with chitosan/chitosanate or DLPC lipid nanoparticles.

The mucus-penetrating feature may be a mucus-penetrating surface modification of the delivery vehicle, such as a mucus-penetrating surface modification of a nanoparticle. The surface modification may be one or more of polyethylene glycol, poly (2-alkyl-2-oxazoline), poly(2-ethyl-2-oxazoline), poly(2-n-propyl-2-oxazoline), and poly(2-methyl-2-oxazaline), a salt thereof, a di block polymer and a tri block polymer thereof. In some embodiments, the polyethylene glycol surface modification has an average molecular weight ranging from about 2000 Da to about 3000 Da. In some embodiments, the surface modification is a compound of

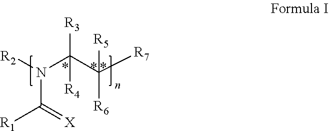

disclosed in PCT/US17/61111, which is incorporated by reference herein in its entirety. In some embodiments, a delivery vehicle includes more than one mucus-penetrating feature selected from a zwitterionic feature, a mucus-penetrating lipid composition that confers properties to the delivery vehicle and a mucus-penetrating surface modification, and combinations thereof.

The compositions herein include those where the MPP is conjugated directly to the delivery vehicle. In other embodiments, the MPP is indirectly conjugated to the delivery vehicle. The MPP may be conjugated directly to the surface modification, covalently or non-covalently.

The MPPs for use in the compositions and with the methods provided herein are cell-penetrating peptides (CPPs) that additionally have at least one mucus-penetrating feature. The vast majority of CPPs are not MPPs. Previously known CPPs generally fall into three group of peptides: cationic, amphipathic and hydrophobic. Due to the physical properties of the mucus, these CPPs will adhere to the mucus. For the simultaneous purposes of mucus penetration and cell penetration, a new class of peptides is provided herein referred to as mucus-penetrating peptides (MPPs). When conjugated, specifically, to a mucus penetrating delivery system will confer upon the delivery system a unique and improved ability to enter the underlying epithelial cells in the presence of physiologically relevant mucus.

The MPPs for use in the compositions and with the methods provided herein have characteristics that confer mucus-penetrating properties. In some embodiments, the MPPs herein have an average hydropathy of an amino acid sequence of the MPP as measured by a Hodges score of less than or equal to 10 at pH 7. In some cases, an average hydropathy of an amino acid sequence of an MPP as measured by a Fauchere score can be less than or equal to 0.5 at pH 7. In some embodiments, the MPP is between 3 and 100 amino acids. In some embodiments, an MPP has an amino acid sequence wherein no more than 40% of the amino acids of the MPP sequence has a Hodges score greater than 10. In some cases, a net charge of an MPP can be from about +2 to about −2. In some cases, a net charge of an MPP can be less than about +2. The MPP may have one or more cysteines. In some cases, if the peptide comprises one of more cysteines, the cysteine does not contain a free thiol.

In some embodiments an MPP is one of SEQ ID Nos. 1-35 and SEQ ID No. 36 provides a positive mucus binding control for hydrophobic peptides. In other embodiments, the MPP has an amino acid sequence at least about 80% homology, 90% homology, 95% homology, 98% homology or 99% homologous with any one of SEQ ID Nos. 1-35 and in addition has at least one mucus penetrating features including (a) an average hydropathy of an amino acid sequence of the MPP as measured by a Hodges score of less than or equal to 10 at pH 7; (b) average hydropathy of an amino acid sequence of an MPP as measured by a Fauchere score of less than or equal to 0.5 at pH 7. (c) 3 to 100 amino acids in length; (d) an amino acid sequence wherein no more than 40% of the amino acids of the MPP sequence has a Hodges score greater than 10; (e) a net charge of an MPP can be from about +2 to about −2; (0 one or more cysteines, where the cysteine does not contain a free thiol. In some embodiments, an MPP is one of SEQ ID Nos. 1, 4, 5, 6, 7, 14, 20, 21, 22 and 29.

The compositions herein include a cargo. A cargo can include a polynucleic acid, a dye, a drug, a protein, a lipid nanoparticle, or a chemical agent. In some cases, the cargo is a nucleic acid, including without limitation single-stranded, double-stranded or partially double-stranded nucleic acid, RNA, DNA and RNA-DNA hybrids. A cargo can comprise an isolated and purified circular polynucleic acid. The nucleic acid of a cargo may encode for a protein or biologically active portion of a protein. In some embodiments, cargo such as a nucleic acid encoding a protein is directed to the gastro-intestinal (GI) tract. In some embodiments, cargo such as a nucleic acid encoding a protein is directed to treating a disease or condition in the gastro-intestinal (GI) tract. In some embodiments, the encoded protein is all or a portion of adenomatous polyposis coli (APC), defensin (HD-5), or defensin alpha 6 (HD-6).

In some embodiments, the cargo is contained entirely within a delivery vehicle such as a nanoparticle. In some embodiments, the cargo is partially contained within the delivery vehicle. For example, in some cases, the cargo is a polynucleic acid and the isolated and purified circular polynucleic acid can be at least partially encapsulated in the delivery vehicle. In some cases, an isolated and purified circular polynucleic acid can be completely encapsulated in the delivery vehicle. In some cases, an isolated and purified polynucleic acid, such as DNA, RNA, circular or linear nucleic acid can encode a protein that is active in a gastrointestinal tract or an active fragment thereof. In some cases, a protein comprises adenomatous polyposis coli, (3-galactosidase, defensin alpha 5, defensin alpha 6, or any combination thereof. In some cases, an isolated and purified polynucleic acid, such as DNA, RNA, circular or linear nucleic acid can encode a protein or an active fragment thereof that is active outside the gastrointestinal tract.

Disclosed herein are pharmaceutical compositions comprising a delivery vehicle disclosed herein and at least one of: an excipient, a diluent, or a carrier.

Disclosed herein are methods of making a delivery vehicle. The methods include selecting a peptide with cell-penetrating and mucus-penetrating properties; selecting a delivery vehicle with at least one mucus-penetrating property; and conjugating, indirectly or directly, the peptide and the delivery vehicle.

In some embodiments of the method, the peptide is an MPP that has one or more of the following features: a) an average hydropathy of an amino acid sequence of the MPP as measured by a Hodges score of less than or equal to 10 at pH 7; (b) average hydropathy of an amino acid sequence of an MPP as measured by a Fauchere score of less than or equal to 0.5 at pH 7; (c) 3 to 100 amino acids in length; (d) an amino acid sequence wherein no more than 40% of the amino acids of the MPP sequence has a Hodges score greater than 10; (e) a net charge of an MPP can be from about +2 to about −2; (0 one or more cysteines, where the cysteine does not contain a free thiol. In some embodiments, the MPP is selected from one or more of SEQ ID Nos. 1-35. In other embodiments, the MPP has an amino acid sequence at least about 80% homology, 90% homology, 95% homology, 98% homology or 99% homologous with any one of SEQ ID Nos. 1-35 and in addition has at least one mucus penetrating features including (a) an average hydropathy of an amino acid sequence of the MPP as measured by a Hodges score of less than or equal to 10 at pH 7; (b) average hydropathy of an amino acid sequence of an MPP as measured by a Fauchere score of less than or equal to 0.5 at pH 7; (c) 3 to 100 amino acids in length; (d) an amino acid sequence wherein no more than 40% of the amino acids of the MPP sequence has a Hodges score greater than 10; (e) a net charge of an MPP can be from about +2 to about −2; (f) one or more cysteines, where the cysteine does not contain a free thiol. In some embodiments, an MPP is one of SEQ ID Nos. 1, 4, 5, 6, 7, 14, 20, 21, 22 and 29.

In the methods provided herein, the MPP or a portion thereof is exposed on the surface of the conjugate. In some embodiments, the delivery vehicle used in the methods is a nanoparticle. The nanoparticle may be a lipid-containing nanoparticle, such as a liposome, a liposomal polyplex, or a lipoplex. In some embodiments of the method, the nanoparticle includes a mucus-penetrating surface modification. In some embodiments, the surface modification is polyethylene glycol. poly (2-alkyl-2-oxazoline), poly(2-ethyl-2-oxazoline), poly (2-propyl-2-oxazoline), and poly(2-methyl-2-oxazaline), a salt thereof, a di block polymer and a tri block polymer thereof. In some embodiments, the polyethylene glycol surface modification has an average molecular weight ranging from about 2000 Da to about 3000 Da. In some embodiments, the surface modification is a compound of Formula I disclosed in PCT/US17/61111, which is incorporated by reference herein in its entirety.

In the methods provided herein, the delivery vehicle may include a cargo such as a polynucleic acid, a dye, a drug, a protein, a liposome, or a chemical agent. In some cases, the cargo is a nucleic acid, including without limitation single stranded, double-stranded or partially double stranded nucleic acid, RNA, DNA and RNA-DNA hybrids. A cargo can comprise an isolated and purified circular polynucleic acid. The nucleic acid of a cargo may encode for a protein or biologically active portion of a protein. In some embodiments, the encoded protein is all or a portion of adenomatous polyposis coli (APC), defensin (HD-5), or defensin alpha 6 (HD-6).

Disclosed herein are methods of treatment comprising administering the compositions disclosed herein to a subject in need thereof. The method provided herein include methods of treating a disease or condition characterized by having at least one tissue targeted for therapy wherein the tissue comprises a layer of mucus, and administering the compositions described herein. In some cases, a target of a treatment can comprise an eye, intestine, colon, lung, small intestine, intestinal tract, or cervix. In some cases, a subject has a disease selected from the group consisting of familial polyposis (FAP), attenuated FAP, colorectal cancer, chronic inflammatory bowel disease, chronic inflammatory bowel disease, microvillus inclusion disease, a congenital diarrhea condition or disease and any combination thereof.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure can be utilized, and the accompanying drawings of which:

FIG. 1 shows a representative cell-penetration assay (using Caco-2 cells) in which arbitrary fluorescence units are shown for peptides having the sequence of SEQ ID Nos: 28, 36 or 37 conjugated to FITC, compared against a negative control.

FIG. 2 shows a cell-penetration assay (% intensity in a dynamic light scattering (DLS) measurement) using an exemplary base system (30/60/10 MVL5/DOPC/Chol), in the presence or absence of mucin.

FIG. 3 shows a cell-penetration assay (% intensity in a DLS measurement) using an exemplary base system (5% DSPE-SS-PEG), in the presence or absence of mucin.

FIG. 4 shows a cell-penetration assay (% intensity in a DLS measurement) using SEQ ID NO. 36 conjugated systems in the presence of absence of mucin.

FIG. 5 shows a cell-penetration assay (% intensity in a DLS measurement) using SEQ ID NO. 1 conjugated systems in the presence or absence of mucin.

FIG. 6 shows a cell-penetration assay (% intensity in a DLS measurement) using SEQ ID NO. 2 conjugated systems in the presence of mucin.

FIG. 7 shows a cell-penetration assay (% intensity in a DLS measurement) using SEQ ID NO. 3 conjugated systems in the presence of mucin.

FIG. 8 shows a cell-penetration assay (% intensity in a DLS measurement) using SEQ ID NO. 4 conjugated systems in the presence or absence of mucin.

FIG. 9 shows a cell-penetration assay (% intensity in a DLS measurement) using SEQ ID NO. 5 conjugated systems in the presence or absence of mucin.

FIG. 10 shows a cell-penetration assay (% intensity in a DLS measurement) using SEQ ID NO. 6 conjugated systems in the presence or absence of mucin.

FIG. 11 shows a cell-penetration assay (% intensity in a DLS measurement) using SEQ ID NO. 7 conjugated systems in the presence or absence of mucin.

FIG. 12 shows a cell-penetration assay (% intensity in a DLS measurement) using SEQ ID NO. 8 conjugated systems in the presence or absence of mucin.

FIG. 13 shows a cell-penetration assay (% intensity in a DLS measurement) using SEQ ID NO. 9 conjugated systems in the presence or absence of mucin.

FIG. 14 shows a cell-penetration assay (% intensity in a DLS measurement) using SEQ ID NO. 10 conjugated systems in the presence or absence of mucin.

FIG. 15 shows a cell-penetration assay (% intensity in a DLS measurement) using SEQ ID NO. 12 conjugated systems in the presence or absence of mucin.

FIG. 16 shows a cell-penetration assay (% intensity in a DLS measurement) using SEQ ID NO. 13 conjugated systems in the presence or absence of mucin.

FIG. 17 shows a cell-penetration assay (% intensity in a DLS measurement) using SEQ ID NO. 14 conjugated systems in the presence or absence of mucin.

FIG. 18 shows a cell-penetration assay (% intensity in a DLS measurement) using SEQ ID NO. 15 conjugated systems in the presence or absence of mucin.

FIG. 19 shows a cell-penetration assay (% intensity in a DLS measurement) using SEQ ID NO. 16 conjugated systems in the presence or absence of mucin.

FIG. 20 shows a cell-penetration assay (% intensity in a DLS measurement) using SEQ ID NO. 17 conjugated systems in the presence or absence of mucin.

FIG. 21 shows a cell-penetration assay (% intensity in a DLS measurement) using SEQ ID NO. 19 conjugated systems in the presence or absence of mucin.

FIG. 22 shows a cell-penetration assay (% intensity in a DLS measurement) using SEQ ID NO. 20 conjugated systems in the presence or absence of mucin.

FIG. 23 shows a cell-penetration assay (% intensity in a DLS measurement) using SEQ ID NO. 21 conjugated systems in the presence or absence of mucin.

FIG. 24 shows cell-penetration assay (% intensity in a DLS measurement) using SEQ ID NO. 22 conjugated systems in the presence or absence of mucin.

FIG. 25 shows a cell-penetration assay (% intensity in a DLS measurement) using SEQ ID NO. 23 conjugated systems in the presence or absence of mucin.

FIG. 26 shows a cell-penetration assay (% intensity in a DLS measurement) using SEQ ID NO. 24 conjugated systems in the presence or absence of mucin.

FIG. 27 shows a cell-penetration assay (% intensity in a DLS measurement) using SEQ ID NO. 26 conjugated systems in the presence or absence of mucin.

FIG. 28 shows a cell-penetration assay (% intensity in a DLS measurement) using SEQ ID NO. 32 conjugated systems in the presence or absence of mucin.

FIG. 29 shows a cell-penetration assay (% intensity in a DLS measurement) using SEQ ID NO. 34 conjugated systems in the presence or absence of mucin.

FIG. 30 shows a representative plot for peptides analyzed according to their hydropathy scores using the Hodges method.

FIG. 31 shows mucus penetration of a SEQ ID No. 1 coupled lipid nanoparticle, compared to a lipid nanoparticle without SEQ ID No. 1.

FIGS. 32A-32C show distribution of lipid nanoparticles at the surface of intestinal epithelial cells. Lipid nanoparticles containing no coupled peptide are shown in FIG. 32A; Lipid nanoparticles coupled with the peptide of SEQ ID No. 37 are shown in FIG. 32B; and Lipid nanoparticles coupled with the peptide of SEQ ID No. 29 are shown in FIG. 32C.

FIG. 33 shows the results of a large screen cell penetration assay using various exemplary mucus-penetrating peptides of this disclosure (SEQ ID Nos. 1-21), a Pos-Tat peptide (SEQ ID No. 37), a vehicle control (DMSO), and a negative control.

FIG. 34 shows a cell-penetration assay using mucin.

### DETAILED DESCRIPTION OF THE DISCLOSURE

The following description and examples illustrate embodiments of the disclosure in detail. It is to be understood that this disclosure is not limited to the particular embodiments described herein and as such can vary. Those of skill in the art will recognize that there are numerous variations and modifications of the disclosure, which are encompassed within its scope.

### Definitions

The term “about” and its grammatical equivalents in relation to a reference numerical value and its grammatical equivalents as used herein can include a range of values plus or minus 10% from that value. For example, the amount “about 10” includes amounts from 9 to 11. The term “about” in relation to a reference numerical value can also include a range of values plus or minus 10%, 9%, 8%, 7%, 6%, 5%, 4%, 3%, 2%, or 1% from that value.

The term “administering” and its grammatical equivalents can refer to any method of providing a structure described herein to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, and parenteral administration, including injectable such as intravenous administration, intra-arterial administration, intramuscular administration, and subcutaneous administration. Administration can be continuous or intermittent. In various aspects, a structure disclosed herein can be administered therapeutically. In some instances a structure can be administered to treat an existing disease or condition. In further various aspects, a structure can be administered prophylactically to prevent a disease or condition.

The term “biodegradable” and its grammatical equivalents can refer to polymers, compositions and formulations, such as those described herein that are intended to degrade during use. The term “biodegradable” is intended to cover materials and processes also termed “bioerodible.”

The term “cancer” and its grammatical equivalents as used herein can refer to a hyperproliferation of cells whose unique trait—loss of normal controls—results in unregulated growth, lack of differentiation, local tissue invasion, and metastasis. With respect to the inventive methods, the cancer can be any cancer, including any of acute lymphocytic cancer, acute myeloid leukemia, alveolar rhabdomyosarcoma, bladder cancer, bone cancer, brain cancer, breast cancer, cancer of the anus, anal canal, rectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, Hodgkin lymphoma, hypopharynx cancer, kidney cancer, larynx cancer, leukemia, liquid tumors, liver cancer, lung cancer, lymphoma, malignant mesothelioma, mastocytoma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, stomach cancer, testicular cancer, thyroid cancer, ureter cancer, and/or urinary bladder cancer. As used herein, the term “tumor” refers to an abnormal growth of cells or tissues, e.g., of malignant type or benign type.

The term “cargo” as used herein can refer to one or more molecules or structures encompassed in a delivery vehicle for delivery to or into a cell or tissue. Non-limiting examples of cargo include a nucleic acid, a dye, a drug, a protein, a nanoparticle, a small chemical molecule and any combinations thereof.

The term “cell” and its grammatical equivalents as used herein can refer to a structural and functional unit of an organism. A cell can be microscopic in size and can consist of a cytoplasm and a nucleus enclosed in a membrane. A cell can refer to an intestinal crypt cell. A crypt cell can refer to the crypts of Lieberkuhn which are pit-like structures that surround the base of the villi in the intestine. A cell can be of human or non-human origin.

A “chemotherapeutic agent” or “Chemotherapeutic compound” and their grammatical equivalents as used herein, can be a chemical compound useful in the treatment of a disease, for example cancer.

“Conjugate” as used herein refers to the association, covalently or non-covalently of two or more molecules or structures, including without limitation, the association of a peptide, such as a mucus-penetrating peptide (MPP) with a delivery vehicle, a polymer and/or a surface modification.

The term “function” and its grammatical equivalents as used herein can refer to the capability of operating, having, or serving an intended purpose. Functional can comprise any percent from baseline to 100% of an intended purpose. For example, functional can comprise or comprise about 5, 10, 15, 20, 25, 30, 35, 40, 45, 50, 55, 60, 65, 70, 75, 80, 85, 90, 95, or up to about 100% of an intended purpose. In some cases, the term functional can mean over or over about 100% of normal function, for example, 125, 150, 175, 200, 250, 300%, 400%, 500%, 600%, 700% or up to about 1000% of an intended purpose.

The term “hydrophilic” and its grammatical equivalents as used herein refers to substances or structures that have polar groups that readily interact with water.

The term “hydrophobic” and it's grammatical equivalents as used herein refers to substances or structures that have polar groups that do not readily interact with water.

The term “mucus,” and its grammatical equivalents as used herein, can refer to a viscoelastic natural substance containing primarily mucin glycoproteins and other materials, which protects epithelial surface of various organs/tissues, including but not limited to respiratory, nasal, cervicovaginal, gastrointestinal, rectal, visual and auditory systems.

The term “structure” and its grammatical equivalents as used herein can refer to a nanoparticle or nanostructure. A structure can be a liposomal structure. A structure can also refer to a particle. A delivery vehicle can be a structure. A structure or particle can be a nanoparticle or nanostructure. A particle or structure can be of any shape having a diameter from about 1 nm up to about 1 micron. A nanoparticle or nanostructure can be or can be about 100 to 200 nm. A nanoparticle or nanostructure can also be up to 500 nm. Nanoparticles or nanostructures having a spherical shape can be referred to as “nanospheres”.

The term “lipid structure” as used herein encompasses liposomes, lipid nanoparticles and nucleic acid lipoplexes. “Liposomes” as used herein refers to a synthetic structure composed of one or more concentric lipid bilayers. “Nucleic acid lipoplexes” as used herein refers to liposomes that are mixed with nucleic acids to form organized structures (called lipoplexes). “Lipid nanoparticles” as used herein refers to a lipid monolayer enclosing cargo in a lipid core.

The terms “nucleic acid,” “polynucleotide,” and “oligonucleotide” and their grammatical equivalents can be used interchangeably and can refer to a deoxyribonucleotide and/or ribonucleotide polymer, in linear or circular conformation, and in either single- or double-stranded form. For the purposes of the present disclosure, these terms should not to be construed as limiting with respect to length. The terms can also encompass known analogues of natural nucleotides, as well as nucleotides that are modified in the base, sugar and/or phosphate moieties (e.g., phosphorothioate backbones). In general, an analogue of a particular nucleotide can have the same base-pairing specificity, i.e., an analogue of adenine “A” can base-pair with thymine “T”.

The term “pharmaceutically acceptable carrier” and their grammatical equivalents can refer to sterile aqueous or non-aqueous solutions, dispersions, suspensions or emulsions, as well as sterile powders for reconstitution into sterile injectable solutions or dispersions just prior to use. Proper fluidity can be maintained, for example, by the use of coating materials such as lecithin, by the maintenance of the required particle size in the case of dispersions and by the use of surfactants. These solutions, dispersions, suspensions or emulsions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly (orthoesters) and poly (anhydrides).

The term “predisposed” as used herein can be understood to mean an increased probability (e.g., at least 1%, 5%, 10%, 20%, 30%, 40%, 50%, 60%, 70%, 80%, 90%, 100%, 150%, 200%, or more increase in probability) that a subject will suffer from a disease or condition.

The term “promoter” as used herein can be a region of DNA that initiates transcription of a particular gene or portion thereof.

The term “recipient” and their grammatical equivalents as used herein can refer to a subject. A subject can be a human or non-human animal. The recipient can also be in need thereof, such as needing treatment for a disease such as cancer. In some cases, a recipient may be in need thereof of a preventative therapy. A recipient may not be in need thereof in other cases.

The term “risk” and its grammatical equivalent as used herein can refer to the probability that an event will occur over a specific time period and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a subject compared either to the absolute risks of low risk cohorts or an average population risk, which can vary by how clinical risk factors are assessed.

The term “subject” and its grammatical equivalents as used herein can refer to a human or a non-human. A subject can be a mammal. A subject can be a human mammal of a male or female gender. A subject can be of any age. A subject can be an embryo. A subject can be a newborn or up to about 100 years of age. A subject can be in need thereof. A subject can have a disease such as cancer.

The term “sequence” and its grammatical equivalents as used herein can refer to a nucleotide sequence, which can be DNA and/or RNA; can be linear, circular or branched; and can be either single-stranded or double stranded. A sequence can be of any length, for example, between 2 and 1,000,000 or more nucleotides in length (or any integer value there between or there above), e.g., between about 100 and about 10,000 nucleotides or between about 200 and about 500 nucleotides.

“Surface modification”, as used herein can refer to an agent or material which modifies one or more properties of a structure's surface, including, but not limited to, hydrophilicity (e.g., can make a surface more or less hydrophilic), surface charge (e.g., makes a surface neutral or near neutral or more negative or positive), and/or enhances transport in or through bodily fluids and/or tissues, such as mucus. A surface modification agent can be a polymer.

“Mucus-penetrating surface modification” as used herein can refer to a surface modification which has one or more properties which allow it and the structure it modifies to penetrate a naturally-occurring mucus layer of a mammalian cell layer or tissue such as mucus of the colon, lung, eye or cervix.

The term “stem cell” as used herein, can refer to an undifferentiated cell of a multicellular organism that is capable of giving rise to indefinitely more cells of the same type. A stem cell can also give rise to other kinds of cells by differentiation. Stem cells can be found in crypts. Stem cells can be progenitors of epithelial cells found on intestinal villi surface. Stem cells can be cancerous. A stem cell can be totipotent, unipotent or pluripotent. A stem cell can be an induced stem cell.

The terms “treatment” or “treating” and their grammatical equivalents can refer to the medical management of a subject with the intent to cure, ameliorate, stabilize, or prevent a disease, condition, or disorder. Treatment can include active treatment, that is, treatment directed specifically toward the improvement of a disease, condition, or disorder. Treatment can include causal treatment, that is, treatment directed toward removal of the cause of the associated disease, condition, or disorder. In addition, this treatment can include palliative treatment, that is, treatment designed for the relief of symptoms rather than the curing of the disease, condition, or disorder. Treatment can include preventative treatment, that is, treatment directed to minimizing or partially or completely inhibiting the development of a disease, condition, or disorder. Treatment can include supportive treatment, that is, treatment employed to supplement another specific therapy directed toward the improvement of the disease, condition, or disorder. In some instances, a condition can be pathological. In some instances, a treatment may not completely cure, ameliorate, stabilize or prevent a disease, condition, or disorder.

### Overview

Disclosed herein are compositions and methods useful for delivering a cargo for use in treating a disease or condition where delivery to the intended target tissue or cells includes penetration through mucus. The compositions and methods herein can be used, for example, for delivery of a gene therapy, delivery of a therapeutic molecule and for delivery of diagnostic molecules such as dye. The compositions and methods described throughout provide cell-penetrating and mucus-penetrating properties and can be used to deliver a cargo through a mucus layer to and/or into target cells. The compositions and methods herein can be used to provide treatment to cells and tissues with mucus layers such as to the colon, lung, eye and cervix. For example, the compositions and methods herein can be used to provide treatment such as local gene therapy to a site, such as an intestinal crypt cell for diseases and conditions including familial polyposis (FAP), attenuated FAP, colorectal cancer, chronic inflammatory bowel disease, chronic inflammatory bowel disease.

### Mucus Penetrating Cell-Penetrating Peptides (MPPs)

Cell penetrating peptides (CPPs) can be short polypeptides that can allow for increased uptake of drugs into cells. Cell-penetrating peptides can be peptide sequences that cross the cytoplasmic membrane efficiently, however they may be limited in their ability to cross a mucus-layer and reach the underlying cells and tissue.

Mucus-Penetrating Cell-Penetrating Peptides (MPPs) are a novel class of peptides that have cell-penetrating properties and in addition, permit penetration through a layer of mucus such as the naturally-occurring layers of mucus in the colon, lung, eye and cervix. MPP can further be used to target structures, such as liposomal structures, to intracellular components of cells. They can also be designed to specifically target certain cell types. MPPs can be conjugated to nanoparticles to allow penetration of the particles through the mucus layer and also for interaction with cells so as to result in increased penetration or targeting of cells. In some cases, a particle that has an MPP can be internalized into a cell with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particles that does not contain an MPP.

In some embodiments, the delivery vehicle comprises a mucus-penetrating peptide (MPP). The MPP may be conjugated to the delivery vehicle, a surface modification of the delivery vehicle or the cargo, such that the MPP is exposed such that it may come into contact, in whole or in part, with a mucus layer, mucus-containing tissue, organ or extracellular surface. The presence of the MPP confers improved penetration of the delivery vehicle through the mucus (diffusion and/or movement through). In some embodiments, the penetration is improved 2-fold, 3-fold, 4-fold, 5-fold, 6-fold, 7-fold, 8-fold, 9-fold, 10-fold, 15-fold, 20-fold, 25-fold, 30-fold, 50-fold, 100-fold, or more as compared to the delivery of the delivery vehicle and/or cargo that does not the MPP.

Numerous methods of determining the internalization behavior and/or transfection capability of a given MPP peptide are established in the art, for example, by attaching a detectable label (e.g. a fluorescent dye) to the peptide (and/or to the cargo to be transfected) or by fusing the peptide with a reporter molecule, thus enabling detection once cellular uptake of the peptide occurred, e.g., by means of FACS analysis or via specific antibodies. The skilled person is also well aware how to select the respective concentration ranges of the peptide and, if applicable, of the cargo to be employed in such methods, which may depend on the nature of the peptide, the size of the cargo, the cell type used, and the like.

An MPP can have an amino acid sequence having from about 3 to 100 amino acids, including without limitation from about 3 to 5, 5 to 10, 10 to 20, 20 to 40, 30 to 60, or 80 to 100 amino acids. An MPP can have from about 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or up to about 100 amino acids.

An MPP has the ability to penetrate a mucus-layer that overlays or surrounds a target cell or tissue. An MPP can be employed to penetrate the mucus layer of a target tissue such as the colon, lung, eye or cervix of a mammal. MPPs can be conjugated to delivery vehicles, including nanoparticles, to allow penetration of the delivery vehicle through the mucus layer and also for interaction with cells so as to result in increased penetration or targeting of cells. In some cases, a particle that has an MPP permeates a mucus layer with an efficacy of at least about 20%, 25%, 30%, 35%, 40%, 45%, 50%, 55%, 60%, 65%, 70%, 75%, 80%, 85%, 90%, 95%, or up to about 100% as compared to a comparable particles that does not contain an MPP. Numerous methods of determining the penetration of a mucus layer can be used to assess the penetration by an MPP or an MPP conjugated directly or indirectly with a delivery vehicle. In one method, the MPP conjugated to a delivery system carrying a fluorescent labelled cargo can be dropped on top of fresh porcine intestines. The intestines can be embedded, frozen and cryosectioned and mucus penetration analyzed via fluorescent microscopy.

MPPs can be designed to include characteristics that provide for mucus penetration and retain cell-penetration properties. In some cases, an MPP can be designed by considering hydrophilicity. A computational analysis can be used to quantify a degree of hydrophobicity or hydrophilicity of amino acids of a protein. In some cases, amino acid scale can be utilized in a computation analysis to determine a numerical value assigned to each type of amino acid. The most frequently used scales are the hydrophobicity or hydrophilicity scales and the secondary structure conformational parameters scales, but many other scales exist which are based on different chemical and physical properties of the amino acids. Various scales can be utilized to determine hydrophobicity or hydrophilicity for example, Kyte-Doolittle, Hopp-Woods, Eisenberg, Manavalan, Black, Fauchere, Janin, Rao & Argos, Tanford, Welling, Parker, Cowan Rose, Abraham & Leo, Bull & Breese, Guy, Miyazawa, Roseman, Wolfenden, Wilson, Rf mobility, Chothia, and any combination thereof.

In some cases, a Hodges study may be performed to identify a suitable MPP. A Hodges study can take into account intrinsic hydrophilicity or hydrophobicity of amino acid residues in peptides in the absence of nearest-Neighbor or conformational effects. Manifestations of a hydrophobic effect are evident in many facets of peptide structure. These include stabilization of protein globular structure in solution, the presence of amphipathic structures induced in peptides or membrane proteins in lipid environments, and protein—protein interactions associated with a protein subunit assembly, protein—receptor binding, and other intermolecular biorecognition processes. Approaches that can be utilized can include: chromatographic or nonchromatographic. Assays can include, partitioning, accessible surface area calculations, site-directed mutagenesis, physical property measurements, and chromatographic techniques. A partitioning assay can include liquid-liquid partitioning. A site-directed mutagenesis assay can include amino acid substitutions on a surface or within an interior of a protein. A physical property measurement can include surface tension of amino acid solutions, solvations free energy of amino acids, and apparent heat capacity of peptides. Chromatographic techniques can include reverse-phase high performance liquid chromatography (RP-HPLC). Using this RP-HPLC-based approach, a regression analysis of a random collection of peptides to relate peptide hydrophobicity to peptide retention behavior can be performed. In some cases, RP-HPLC can be applied to the separation of mixtures of synthetic model peptides with just single amino acid substitutions in a defined peptide sequence. RP-HPLC can be applied to the separation of mixtures of de novo designed model peptides with a particular sequence, where an X amino acid can be substituted by all naturally occurring amino acids and norvaline, norleucine, and ornithine. From the observed retention behavior of these model peptides, one can obtain intrinsic hydrophilicity/hydrophobicity values of the amino acid side chains at pH 2, 5, and 7 (the latter in the presence and absence of salts).

In some cases, to determine intrinsic hydrophilicity/hydrophobicity values for amino acid side chains in peptides/proteins, several criteria can be considered: (1) the model peptide sequence should have a reduced tendency to form any type of secondary structure (α-helix, β-sheet, or (3-turn) in any environment (aqueous or hydrophobic) that could restrict the interaction of the substitution site with the hydrophobic matrix during partitioning of the peptide between the mobile phase and stationary phase during RP-HPLC; (2) the peptide should be of sufficient length to ensure multisite binding; (3) the peptide should be of sufficient overall hydrophobicity to allow the substitution of all naturally occurring amino acid side chains while maintaining satisfactory retention behavior; (4) the distribution of amino acid side chains should be such that there is reduced clustering of hydrophobic side chains that may minimize the contribution of the substituting amino acid side chain; (5) the peptide should be long enough to maintain satisfactory retention behavior on substituting the amino acids but not so long as to diminish the full expression of the hydrophilicity/hydrophobicity of the substituted amino acid due to a chain length effect (generally for peptides >15 residues) on peptide retention times 65; (6) the substitution site should be next to a residue that has a minimal side chain in terms of size and hydrophobicity, thus allowing the substituting amino acid to express its true intrinsic hydrophilicity/hydrophobicity; and (7) there should be no nearest neighbor effects (i to i±1 interactions with the substituting residue)—such effects can be eliminated if there is free rotation of the bonds represented by the angles ψ(Cα-C) and φ(Cα-N), i.e., there is no steric hindrance between the substituting side chain at position i and its nearest-neighbor side chains at position i±1.

Several parameters can be considered in a computational analysis of a peptide hydrophilicity or hydrophobicity. For example, a window size can be the length of the interval to use for the profile computation, i.e. the number of amino acids examined at a time to determine a point of hydrophobic character. When computing the score for a given residue i, the amino acids in an interval of the chosen length, centered around residue i, are considered. In other words, for a window size n, the i−(n−1)/2 neighboring residues on each side of residue i to compute the score for residue i. The score for residue i is the sum of the scale values for these amino acids, optionally weighted according to their position in the window. One should choose a window that corresponds to the expected size of the structural motif under investigation: A window size of 5 to 7 is appropriate for finding hydrophilic regions that are likely to be exposed on the surface and may potentially be antigenic. Window sizes of 19 or 21 will make hydrophobic, membrane-spanning domains stand out rather clearly (typically >1.6 on the Kyte & Doolittle scale). Another parameter can be the relative weight of the window edges. The central amino acid of the window can have a weight of 100%. By default, the amino acids at the remaining window positions have the same weight, but you can attribute a larger weight (in comparison to the other residues) to the residue at the center of the window by setting the weight value for the residues at the extremities of the interval to a value between 0 and 100%. The decrease in weight between the center and the edges will either be linear or exponential, depending on the setting of the weight variation model option. In some cases, a scale can also be normalized. A scale can be unmodified or modified to normalize the values so that they all fit into the range from 0 to 1. Normalization is useful if you want to compare the results of profiles obtained with different scales, and makes plots with a more uniform appearance.

In some cases, a hydropathy of a peptide can be determined. A hydropathy can be a quantitative assessment based on a peptide amino acid sequence. A hydropathy can be determined by a variety of means. In some cases, a Fauchere score can be used to determine a peptide hydrophilicity or hydrophobicity, as in Table 1.

TABLE 1
Fauchere Amino Acid Hydrophobicity Scale at pH 7 (No salt)

| Amino Acid Abbreviation | Fauchere Score |
|---|---|
| Ala | 0.3 |
| Arg | −1.010 |
| Asn | −0.600 |
| Asp | −0.770 |
| Cys | 1.540 |
| Gln | −0.220 |
| Glu | −0.640 |
| Gly | 0 |
| His | 0.130 |
| Ile | 1.8 |
| Leu | 1.7 |
| Lys | −0.990 |
| Met | 1.230 |
| Phe | 1.79 |
| Pro | 0.720 |
| Ser | −0.04 |
| Thr | 0.260 |
| Trp | 2.250 |
| Tyr | 0.960 |
| Val | 1.220 |

A Fauchere score can be determined per residue of an amino acid or per peptide.

In some cases, a hydropathy can be determined by a Hodges study. A Hodges study can be utilized to measure hydrophilicity and hydrophobicity of amino acids by placing each amino acid within a 10-aminoacid peptide: Ac-X-G-A-K-G-A-G-V-G-L, where X is the amino acid being tested. A retention time of each peptide can subsequently be measured using reverse-phase HPLC, as in Table 2. In some cases, a Hodges study can be performed at pH 7. A Hodges study can also be performed in acidic or basic conditions such as from pH 1, 2, 3, 4, 5, 6, 7, 8, 9, 10, 11, or 12. A Hodges study can be performed in the presence or absence of salt. In some cases, a Hodges score can be normalized to Glycine. Glycine can have a score of 0. For example, a negative Hodges score can be given to amino acids that are more hydrophilic than Glycine. A Hodges score can be determined per residue of an amino acid or per peptide.

A Fauchere score or Hodges score per peptide can be determined by dividing a total Fauchere score or Hodges score by the number of amino acid residues present in a sequence. In a Fauchere study, a hydrophobicity score can be measured by determining a partition coefficient. In some cases, a peptide can be screened for an average hydropathy per residue score to be lower or equal to 10 as measured by a Hodges study. In some cases, a peptide can be screened for an average hydropathy per residue score to be lower or equal to 0.5 as measured by a Fauchere study. For example, a peptide may contain a hydropathy per residue that can be or can be about 10. For example, a peptide may contain a hydropathy per residue that can be or can be about 20, 19, 18, 17, 16, 15, 14, 13, 12, 11, 10, 9, 8, 7, 6, 5, 4, 3, 2, 1, 0.5, 0.3, 0.2, 0.1, or 0 as measured at pH 7 from a Hodges study or Fauchere study. In some cases, an MPP can contain no more than 4 adjacent residues with a Hodges score greater than 10. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 10. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 9. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 8. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 7. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 6. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 5. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 4. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 3. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 2. In some cases, an MPP can contain no more than 1, 2, 3, 4, 5, 6, 7, 8, 9, 10, 11, 12, 13, 14, 15, 16, 17, 18, 19, or 20 adjacent residues with a Hodges score greater than 1. In some cases, an MPP can contain no more than 1, 2, 3, 4, 5, 6, 7, 8, 9, 10, 11, 12, 13, 14, 15, 16, 17, 18, 19, or 20 adjacent residues with a Fauchere score greater than 0.5. In some cases, an MPP can contain no more than 1, 2, 3, 4, 5, 6, 7, 8, 9, 10, 11, 12, 13, 14, 15, 16, 17, 18, 19, or 20 adjacent residues with a Fauchere score greater than 0.4. In some cases, an MPP can contain no more than 1, 2, 3, 4, 5, 6, 7, 8, 9, 10, 11, 12, 13, 14, 15, 16, 17, 18, 19, or 20 adjacent residues with a Fauchere score greater than 0.3. In some cases, an MPP can contain no more than 1, 2, 3, 4, 5, 6, 7, 8, 9, 10, 11, 12, 13, 14, 15, 16, 17, 18, 19, or 20 adjacent residues with a Fauchere score greater than 0.2. In some cases, an MPP can contain no more than 1, 2, 3, 4, 5, 6, 7, 8, 9, 10, 11, 12, 13, 14, 15, 16, 17, 18, 19, or 20 adjacent residues with a Fauchere score greater than 0.1. In some cases, a peptide can be screened so that the MPP's total Hodges score is below 200, 190, 180, 170, 150, 140, 130, 120, 110, 100, 90, 80, 70, 60, 50, 40, 30, 20, or 10. In some cases, a peptide can be screened so that the MPP's total Fauchere score is below 20, 19, 18, 17, 16, 15, 14, 13, 12, 11, 10, 9, 8, 7, 6, 5, 4, 3, 2, or 1.

TABLE 2
Hydrophilicity/Hydrophobicity Coefficients determined
at 25° C. by RP-HPLC of Model MPPs by Hodges Study.

| | | | | pH 7^{b}, 10 mM PO₄ Buffer | | |
|---|---|---|---|---|---|---|
| | pH 2^{b} | | | | | +50 mM |
| | 20 mM | 20 mM | pH 5^{b} 10 mM | | +50 mM | NaCl |
| Amino Acid | H₃PO₄ Δ | TFA Δ | PO₄ Buffer | No Salt Δ | NaCl | O₄Δ |
| Substitution^{a} | ^{t}R(Gly)^{c} | ^{t}R(Gly) | ^{Δt}R(Gly) | ^{t}R(Gly) | ^{Δt}R(Gly) | ^{t}R(Gly) |
| Trp | 32.3 | 32.4 | 33.2 | 32.9 | 33.0 | 33.7 |
| Phe | 29.1 | 29.1 | 30.1 | 29.9 | 30.1 | 30.8 |
| n-Leu | 24.6 | 24.6 | 25.6 | 25.6 | 25.9 | 26.6 |
| Leu | 23.4 | 23.3 | 24.1 | 24.2 | 24.6 | 25.1 |
| Ile | 21.3 | 21.4 | 22.2 | 22.4 | 22.8 | 23.0 |
| Met | 16.1 | 15.7 | 16.4 | 16.3 | 17.3 | 16.8 |
| n-Val | 15.4 | 15.2 | 15.9 | 16.3 | 16.9 | 16.8 |
| Tyr | 15.4 | 14.7 | 15.2 | 15.4 | 16.0 | 15.1 |
| Val | 13.8 | 13.4 | 14.0 | 14.4 | 15.0 | 14.6 |
| Pro | 9.4 | 9.0 | 9.4 | 9.7 | 10.4 | 9.9 |
| Cys | 8.1 | 7.6 | 7.9 | 8.3 | 9.1 | 8.2 |
| Ala | 3.6 | 2.8 | 3.3 | 3.9 | 4.1 | 3.4 |
| Glu^{d} | 3.6 | 2.8 | −0.5 | −0.9 | −0.4 | −7.1 |
| Thr | 2.8 | 2.3 | 2.8 | 3.9 | 4.1 | 2.5 |
| Asp | 2.2 | 1.6 | −1.0 | −0.9 | −0.8 | −7.6 |
| Gln | 0.5 | 0.6 | 0.6 | 0.5 | 1.6 | 0.0 |
| Ser | 0.0 | 0.0 | 0.0 | 0.5 | 1.2 | 30.5 |
| Asn | 0.0 | −0.6 | 0.0 | 0.5 | 1.0 | 30.8 |
| Gly | 0.0 | 0.0 | 0.0 | 0.0 | 0.0 | 0.0 |
| Arg | −5.0 | 0.6 | −3.7 | 3.9 | 4.1 | 6.4 |
| His | −7.0 | 0.0 | −5.1 | 3.4 | 4.7 | 3.4 |
| Lys | −7.0 | 2.8 | −3.7 | −1.1 | −2.0 | 3.4 |
| Orn | −7.6 | −0.6 | −6.8 | −3.6 | −2.0 | 2.1 |

^{a}The L-amino acid substitutions at position X in the peptide sequence Ac-X-G-A-K-G-A-G-V-G L-amide; n-Leu, n-Val, and Orn denote norleucine, norvaline, and ornithine, respectively.

A peptide can be screened so that no more than 4 residues with a Hodges score greater than 10 are placed adjacent to each other. A peptide can be screened so that the total number of amino acids with a Hodges score greater than 10 do not account for greater than 40% of the total length of the peptide. In some cases, a CPP can be designed such that a total number of amino acids with a Hodges score greater than about 10 is 40%, 50%, 60%, 70%, 80%, 90%, 95%, 96%, 97%, 98%, 99%, or up to about 100% of the total length of the MPP peptide. In some cases, if an MPP contains a cysteine a thiol group may not be free. In some cases, a net charge of an MPP can be or can be about between −5 to +5. In some cases, a net charge of an MPP can be or can be about between −4 to +4. In some cases, a net charge of an MPP can be or can be about between −3 to +3. In some cases, a net charge of an MPP can be or can be about between −2 to +2. In some cases, a net charge of an MPP can be or can be about between −1 to +1.

In some cases, an MPP may be screened to meet certain characteristics, for example: (P/N/U)0-2(U/H)3-4(P/N/U)0-2; Where −2<=P-N=>2, and where H is a hydrophobic residue, P is a positively charged residue, U is an uncharged polar residue, and N is a negatively charged residue.

In some cases, an MPP may be screened to meet certain characteristics, for example: ((U0-15 (H0-4U1-15))0-15 (P/N)(U0-15 (H0-4U1-15)0-15))1-15; Where −2<=P-N=>2; Length <50; H0-4 indicates no hydrophobic stretches >4, and where H is a hydrophobic residue, P is a positively charged residue, U is an uncharged polar residue, and N is a negatively charged residue.

In some cases, an MPP may be screened and/or confirmed by a functional assay. For example, the MPP conjugated to a delivery system carrying a fluorescent labelled cargo can be dropped on top of fresh porcine intestines. The intestines can be embedded, frozen and cryosectioned and mucus penetration analyzed via fluorescent microscopy. In some cases, an MPP may be screened and/or confirmed by a bench-top assay such as a transwell assay, or by an in vivo mucus-penetration assay.

TABLE 3
In silico screened Mucus-Penetrating peptides
(MPPs). One letter code used. L-amino acids are
in upper case, D-amino acids in lower case.
Repetitions are written in parenthesis.
SEQ ID Nos. 36 and 37 are controls
(not from in silico screening).

| SEQ ID | |
|---|---|
| NO: | Sequence |
| 1 | MATKGGTVKA |
| 2 | MAKPAQGAKY |
| 3 | MSVTGGKMAP |
| 4 | TPKTMTQTYDFS |
| 5 | NSGTMQSASRAT |
| 6 | QAASRVENYMHR |
| 7 | KTIEAHPPYYAS |
| 8 | EPDNWSLDFPRR |
| 9 | NYTTYKSHFQDR |
| 10 | YPYDANHTRSPT |
| 11 | DPATNPGPHFPR |
| 12 | HPGSPFPPEHRP |
| 13 | TSHTDAPPARSP |
| 14 | RQSAGVL |
| 15 | STSTVSTPVPPPVDDTTWLQSAS |
| 16 | RQSAGVLGFAPTNIDDTSFHA |
| 17 | RQWVGDRA |
| 18 | RQSVLDSWGG |
| 19 | RWQVGDRADGE |
| 20 | VGDDSGGFSTTVSTEQNVPDPQV |
| 21 | ADDLENVNEGMRIH |
| 22 | LSTAADMQGVVTDGMASGLDKDYLKPDD |
| 23 | PSSSSSSRIGDP |
| 24 | DPVDTPNPTRRKPGK |
| 25 | TYRFRGPD |
| 26 | DATDRFHGPDAL |
| 27 | DPKGDPKGVTVTVTVTVTGKGDPKPD |
| 28 | TVDNPASTTNKDKLFAV |
| 29 | TVDNDAPTKRASKLFAV |
| 30 | EHGAMEI |
| 31 | NSDSECPLSHDGYCLHDGVCMYIEALDKYA |
| | CNCVVGYIGERCQYRDLKWWELR |
| 32 | NIENSTLATPLS |
| 33 | NSGTMQSASRAT |
| 34 | TSHTDAPPARSP |
| 35 | AEKVDPVKLNLTLSAAAEALTGLGDK |
| 36 | LIIYRDLISH |
| 37 | GRKKRRQRRRPQ (TAT sequence) |

An MPP described herein can comprise one or more sequences described in Table 3. An MPP provides the ability to penetrate through a naturally occurring mucus layer to reach target tissue or cells. An MPP may have an ability to translocate the plasma membrane and facilitate the delivery of various molecular cargos to the cytoplasm or an organelle of a target cell. An MPP can directly penetrate a cellular membrane. An MPP can use endocytosis-mediated entry into a cell. In some cases, an MPP can use translocation through the formation of a transitory structure. An MPP can have an amino acid sequence having from about 5 to about 10 amino acids, from about 10 amino acids to about 20 amino acids, from about 20 amino acids to about 30 amino acids, from about 30 amino acids to about 40 amino acids, from about 40 amino acids to about 60 amino acids. In some cases, an MPP can have from about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or up to about 99 amino acids or greater. Preferably, an MPP can comprise natural amino acids, amino acid derivatives, D-amino acids, modified amino acids, β-amino acid derivatives, α,α-substituted amino acid derivatives, N-substituted a-amino acid derivatives, aliphatic or cyclic amines, amino- and carboxyl-substituted cycloalkyl derivatives, amino- and carboxyl-substituted aromatic derivatives, γ-amino acid derivatives, aliphatic a-amino acid derivatives, diamines and polyamines. Further modified amino acids are known to the skilled artisan.

An amino acid residue of an MPP can be in an L-isomer configuration. In some embodiments, one or more, amino acid residues of an MPP can be present as D-isomers.

An MPP can facilitate cellular uptake of delivery vehicles such as nanoparticles. The delivery vehicle can include small chemical molecules and macromolecules, such as nucleic acids, peptides, proteins, drugs, liposomes, and combinations thereof. An MPP will be exposed to a surface of the delivery vehicle in whole or in part and the MPP will confer the ability to penetrate a mucus layer such that the delivery vehicle conjugated directly or indirectly to the MPP can also penetrate the mucus layer and reach the target cell or tissue.

In some embodiments, the delivery vehicle includes a mucus-penetrating feature such as through a surface modification and the conjugated MPP confers an improved ability of the delivery vehicle to penetrate the mucus layer and provides a targeting to the cell or tissue for the intended therapy and/or diagnostic.

An MPP may be derived from a viral source. For example, a sequence from a poliovirus VP1 BC loop can be TVDNPASTTNKDKLFAV, which has been shown to interact with the Poliovirus Receptor can be utilized or can also be utilized as a template to engineer peptides that retain an ability to penetrate cells and also engineered to include at least one mucus-penetrating feature described herein. In some cases, an MPP for use with the compositions and methods herein includes a sequence disclosed in Table 3 or from about 50%, 60%, 70%, 80%, 90%, 95%, 96%, 97%, 98%, 99%, or up to about 100% homology to a sequence disclosed in Table 3.

In further embodiments, the MPPs used in the present invention do not exert significant cytotoxic and or immunogenic effects to their respective target cells after having been internalized, that is, they do not interfere with cell viability (at least at concentrations that are sufficient to mediate cellular transfection and/or penetration).

Delivery Vehicles

A delivery vehicle for use in the compositions and with the methods herein may include a nanoparticle.

A delivery vehicle can have diameters from about 40 nm, 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, 250 nm, or up to about 550 nm. A delivery vehicle described herein can be a liposomal structure. A liposomal structure can be a vesicle in some cases. A vesicle can be unilamellar or multilamellar. Unilamellar vesicles can comprise a lipid bilayer and generally have diameters from about 50 nm to about 250 nm. Unilamellar vesicles can comprise a lipid bilayer and generally have diameters from about 50 nm, 60 nm, 70 nm, 80 nm, 90 nm, 100 nm, 110 nm, 120 nm, 130 nm, 140 nm, 150 nm, 160 nm, 170 nm, 180 nm, 190 nm, 200 nm, 210 nm, 220 nm, 230 nm, 240 nm, or up to about 250 nm.

The delivery vehicle may include a lipid structure such as a liposome, nucleic acid lipoplex, lipid nanoparticle or other type of lipid structure.

The nanoparticle may include a liposome. A liposome can be a vesicular structure that can form via the accumulation of lipids interacting with one another in an energetically favorable manner. Liposomes can generally be formed by the self-assembly of dissolved lipid molecules, each of which can contain a hydrophilic head group and hydrophobic tails. Liposomes can consist of an aqueous core entrapped by one or more bilayers composed of natural or synthetic lipids. In some cases, liposomes can be highly reactive and immunogenic, or inert and weakly immunogenic. Liposomes composed of natural phospholipids can be biologically inert and weakly immunogenic, and liposomes can possess low intrinsic toxicity.

Unilamellar vesicles can contain a large aqueous core and can be preferentially used to encapsulate drugs. In some cases, a unilamellar vesicle can partially encapsulate a drug. Multilamellar vesicles can comprise several concentric lipid bilayers in an onion-skin arrangement and have diameters from about 1-5 Onion-skin arrangements can have diameters from about 1 μm, 1.5 μm, 2.0 μm, 2.5 μm, 3 μm, 3.5 μm. 4 μm, 4.5 μm, or up to 5.0 μm or greater. Liposomal structures for use with the compositions and methods herein can include a liposome, a lipoplex, or a lipopolyplex, including liposomal structures described in PCT/US17/61111 which is incorporated by reference in its entirety herein.

The compositions and methods herein can include a cargo carried by the delivery vehicle to the target cell or tissue. A cargo may be a cargo comprises a nucleic acid, a dye, a drug, a protein, a nanoparticle, a protein, a small chemical molecule, a chemical agent or any combination thereof. In some cases, the cargo is a nucleic acid that encodes for a protein or biologically active portion of a protein such as adenomatous polyposis coli (APC), defensin (HD-5), and defensin alpha 6 (HD-6). In some cases, the cargo includes a nucleic acid encompassed in a nanoparticle such as a complex of nucleic acid and protamine.

In some cases, a cargo such as a nucleic acid can be fully encapsulated in a delivery vehicle. Full encapsulation can indicate that a cargo in a delivery vehicle may not be significantly degraded after exposure to serum or a nuclease or protease assay that would significantly degrade free cargo such as a DNA, RNA, or protein. In a fully encapsulated system, preferably less than about 25% of a cargo in a delivery vehicle can be degraded in a treatment that would normally degrade 100% of free cargo, more preferably less than about 10%, and most preferably less than about 5% of a cargo in a delivery vehicle can be degraded. In the context of polynucleic acids, full encapsulation may be determined by an Oligreen® assay. Oligreen® is an ultra-sensitive fluorescent nucleic acid stain for quantitating oligonucleotides and single-stranded DNA or RNA in solution (available from Invitrogen Corporation; Carlsbad, Calif.). “Fully encapsulated” can also indicate that a delivery vehicle may be serum-stable, that is, that the delivery vehicle does not rapidly decompose into its component parts upon in vivo administration.

In certain applications, it may be desirable to release a moiety (cargo or portion thereof) once a cargo has entered a cell. A moiety can be utilized to identify a number of cells that have received a cargo. A moiety can be an antibody, dye, scFv, peptide, glycoprotein, carbohydrate, ligand, polymer, a nucleic acid, to name a few. A moiety can be in contact with a linker. A linker can be non-cleavable. Accordingly, in some cases, a linker can be a cleavable linker. This may enable a moiety to be released from a delivery vehicle once contact to a target cell has been made. This may be desirable when a moiety has a greater therapeutic effect when separated from a delivery vehicle. In some cases, a moiety may have a better ability to be absorbed by an intracellular component of a cell, such as an intestinal crypt cell or intestinal crypt stem cell, when separated from a delivery vehicle. In some cases, a linker may comprise a disulfide bond, acyl hydrazone, vinyl ether, orthoester, or a N—PO3.

Accordingly, it may be necessary or desirable to separate a moiety from a delivery vehicle so that a moiety can enter an intracellular compartment. Cleavage of a linker releasing a moiety may be as a result of a change in conditions within a cell as compared to outside cells, for example, due to a change in pH within a cell. Cleavage of a linker may occur due to the presence of an enzyme within a cell which cleaves a linker once a drug, such as a polynucleic acid, enters a cell. Alternatively, cleavage of a linker may occur in response to energy or a chemical being applied to the cell. Examples of types of energies that may be used to effect cleavage of a linker include, but are not limited to light, ultrasound, microwave and radiofrequency energy. In some cases, a linker may be a photolabile linker. A linker used to link a complex may also be an acid labile linker. Examples of acid labile linkers include linkers formed by using cis-aconitic acid, cis-carboxylic alkatriene, polymaleic anhydride, and other acidlabile linkers.

Exemplary Lipids for Use with Delivery Vehicles

The lipids for inclusion into the delivery vehicles herein can include cationic and non-cationic lipids, and can include saturated and unsaturated cationic and non-cationic lipids. The lipid composition of the delivery vehicle may provide improved or increased penetration through mucus. In some embodiments, a delivery vehicle includes a cationic lipid. In some embodiments, a delivery vehicle includes a noncationic lipid. In some embodiments, a delivery vehicle includes both a cationic lipid and a noncationic lipid. In some embodiments, a delivery vehicle includes 1, 2, 3, 4 or more types of lipids selected from one or more of saturated cationic and unsaturated cationic and non-cationic saturated and non-cationic unsaturated lipids.

Saturated non-cationic lipids for use with the delivery vehicles herein include, for example, di-glycerol tetraether phospholipids, sphingoids, ceramides and phosphosphingolipids such as 1,2-Dialkyl-sn-glycero-3-phosphocholine, 1,2-dialkyl-sn-glycero-3-phosphoethanolamine, 1,2-Diaklyl-sn-glycero-3-phosphorylglycerol, 1,2-dialkyl-sn-glycero-3-Phosphatidylserine, 1,2-dialkyl-sn-glycero-3-Phosphate, Monoglycerol alkylate, Glyceryl hydroxyalkylate, Sorbitan monoalkylated, 1,2-dialkyl-sn-glycero-3-phosphoethanolamine-N-methyl, 1,2-dialkyl-sn-glycero-3-phosphomethanol, 1,2-dialkyl-sn-glycero-3-phosphoethanol, 1,2-dialkyl-sn-glycero-3-phosphoethanolamine-N,N-dimethyl, 1,2-dialkyl-sn-glycero-3-phosphopropanol, and 1,2-dialkyl-sn-glycero-3-phosphobutanol, where alkyl means conjugated derivatives of myristic acid, pentadecylic acid, palmitic acid, heptadecanoic acid, stearic acid, lauric acid, tridecylic acid, nonadecylic acid, arachidic acid, heneicosylic acid, behenic acid, tricosylic acid and lignoceric acid.

Unsaturated non-cationic lipids for use with the delivery vehicles herein include, for example, glycerophosphocholines, glycerophosphoethanolamines, glycerophosphoserines, glycerophosphoglycerols, glycerophosphoglycerophosphates, glycerophosphoinositols, glycerophosphoinositol monophosphates, glycerophosphoinositol bisphosphates, glycerophosphoinositol trisphosphates, glycerophosphates, glyceropyrophosphate, glycerophosphoglycerophosphoglycerols, cytidine-5′-diphosphate-glycerols, glycosylglycerophospholipids, glycerophosphoinositolglycans, di-glycerol tetraether phospholipids, sphingoids, ceramides, and phosphosphingolipids, such as 1,2-Dialkyl-sn-glycero-3-phosphocholine, 1,2-dialkyl-sn-glycero-3-phosphoethanolamine, 1,2-Diaklyl-sn-glycero-3-phosphorylglycerol, 1,2-dialkyl-sn-glycero-3-Phosphatidylserine, 1,2-dialkyl-sn-glycero-3-Phosphate, Monoglycerol alkylate, Glyceryl hydroxyalkylate, Sorbitan monoalkylated, 1,2-dialkyl-sn-glycero-3-phosphoethanolamine-N-methyl, 1,2-dialkyl-sn-glycero-3-phosphomethanol, 1,2-dialkyl-sn-glycero-3-phosphoethanol, 1,2-dialkyl-sn-glycero-3-phosphoethanolamine-N,N-dimethyl, 1,2-dialkyl-sn-glycero-3-phosphopropanol and 1,2-dialkyl-sn-glycero-3-phosphobutanol, where alkyl means a conjugated derivative of oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, and α-Linolenic acid.

Saturated cationic lipids for use with the delivery vehicles herein include, for example, those with an alkyl chain greater than 12 carbons in length, generally having a phase transition temperature greater than 20° C.) and being positively charged at pH greater than about 4, such as Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], and N1-[2-((1S)-1-[(3-aminopropyl)amino]-4-[di(3-amino-propyl)amino]butylcarboxamido)ethyl]-3,4-di[alkyl]-benzamide, where alkyl may refer to a conjugated derivative of myristoyl, pentadecenoyl, palmitoyl, heptadecanoyl, stearoyl, lauroyl, tridecanoyl, nonadecanoyl, arachidoyl, heneicasnoyl, behenoyl, tricosanoyl and lignoceroyl.

Unsaturated cationic lipids for use with the delivery vehicles herein include, for example, cationic lipids which are not saturated and are positively charged at a pH greater than about 4, such as Dimethyldioctadecylammonium, 1,2-dialkyl-sn-glycero-3-ethylphosphocholine, 1,2-dialkyl-3-dimethylammonium-propane, 1,2-dialkyl-3-trimethylammonium-propane, 1,2-di-O-alkyl-3-trimethylammonium propane, 1,2-dialkyloxy-3-dimethylaminopropane, N,N-dialkyl-N,N-dimethylammonium, N-(4-carboxybenzyl)-N,N-dimethyl-2,3-bis(alkyloxy)propan-1-aminium, 1,2-dialkyl-sn-glycero-3-[(N-(5-amino-1-carboxypentyl)iminodiacetic acid)succinyl], N1-[2-41 S)-1-[(3-aminopropyl)amino]-4-[di(3-amino-propyl)amino]butylcarboxamido)ethyl]-3,4-di[alkyl]-benzamide, 1,2-Dialkyloxy-N,N-dimethylaminopropane, 4-(2,2-diocta-9,12-dienyl-[1,3]dioxolan-4-ylmethyl)-dimethylamine, O-alkyl ethylphosphocholines, MC3, MC2, MC4, 3β[N-(N′,N′-dimethylaminoethane)-carbamoyl]cholesterol and N4-Cholesteryl-Spermine, where alkyl may refer to a conjugated derivative of oleic acid, elaidic acid, gondoic acid, erucic acid, nervonic acid, mead acid, paullinic acid, vaccenic acid, palmitoleic acid, Docosatetraenoic acid, Arachidonic acid, Dihomo-γ-linolenic acid, γ-Linolenic acid, linolelaidic acid, linoleic acid, Docosahexaenoic acid, Eicosapentaenoic acid, Stearidonic acid, and α-Linolenic acid.

In other cases, an anionic liposome may be used to deliver other therapeutic agents. Anionic lipoplexes can be composed of physiologically safe components including anionic lipids, cations, and DNA. Commonly used lipids in this category are phospholipids that can be found naturally in cellular membranes such as phosphatidic acid, phosphatidylglycerol, and phosphatidylserine

Divalent cations can be incorporated into an anionic liposome system to enable the condensation of nucleic acids prior to envelopment by anionic lipids. Several divalent cations can be used in anionic lipoplexes such as Ca2+, Mg2+, Mn2+, and Ba2+. In some cases, Ca2+ can be utilized in an anionic liposome system.

In some cases, a cationic lipid may attain a positive charge through one or more amines present in a polar head group. In some cases, a liposome can be a cationic liposome. In some cases, a liposome may be a cationic liposome used to carry negatively charged polynucleic acid, such as DNA. In some cases, a cationic (and neutral) lipid may be used for gene delivery.

A cationic lipid can be used to form a liposome. Cationic lipids may commonly attain a positive charge through one or more amines present in the polar head group. A solution of cationic lipids, often formed with neutral helper lipids, can be mixed with DNA to form a positively charged complex termed a lipoplex. Reagents for cationic lipid transfection can include N-[1-(2,3-dioleyloxy)propyl]-N,N,N-trimethylammonium chloride (DOTMA), [1,2-bis(oleoyloxy)-3-(trimethylammonio)propane] (DOTAP), 3β[N-(N′,N′-dimethylaminoethane)-carbamoyl] cholesterol (DC-Chol), and dioctadecylamidoglycylspermine (DOGS). Dioleoylphosphatidylethanolamine (DOPE), a neutral lipid, may often be used in conjunction with cationic lipids because of its membrane destabilizing effects at low pH, which can aide in endolysosomal escape.

A liposome may be formed with neutral helper lipids. A liposome may be generated using cholesterol, N-[1-(2,3-dioleyloxy)propyl]-N,N,N-trimethylammonium chloride (DOTMA), [1,2-bis(oleoyloxy)-3 (trimethylammonio)propane] (DOTAP), 3β[N-(N′,N′-dimethylaminoethane)-carbamoyl] cholesterol (DC-Chol), dioctadecylamidoglycylspermine (DOGS), Dioleoylphosphatidylethanolamine (DOPE), N1-[2-((l S)-1-[(3-aminopropyl)amino]-4-[di(3-amino-propyl)amino]butylcarboxamido)ethyl]-3,4-di[oleyloxy]-benzamide (MVL5), glyceryl mono-oleate (GMO), 1,2-Distearoyl-sn-glycero-3-phosphoethanolamine (DSPE), 1,2-distearoyl-sn-glycero-3-phosphocholine (DSPC), Dimethyldioctadecylammonium (DDAB), a salt thereof, and any combination thereof. Liposomes for use with the compositions and methods herein can be found for example in PCT/US17/61111, which is incorporated herein in its entirety.

### Exemplary Surface Modifications of Delivery Vehicle

Lipids or liposomes or delivery vehicle of the present disclosure may be modified by a surface modification. A surface modification can enhance an average rate at which a delivery vehicle or liposomal structure moves in mucus compared to a comparable delivery vehicle or liposomal structure. A comparable delivery vehicle or liposomal structure may not be surface modified or a comparable liposomal structure may be modified with a polyethylene gycol (PEG) polymer. A modification can facilitate protection from degradation in vivo. A modification may also assist in trafficking of a delivery vehicle or liposome. For example, a modification may allow a delivery vehicle or liposome to traffic within a gastrointestinal (GI) track with an acidic pH due to pH sensitive modifications. A surface modification can also improve an average rate at which a delivery vehicle or liposome moves in mucous. For example, a modification may enhance a rate by 1×, 2×, 3×, 4×, 5×, 6×, 7×, 8×, 9×, 10×, 20×, 30×, 40×, 50×, 60×, 70×, 80×, 90×, 100×, 300×, 500×, 700×, 900×, or up to about 1000× when compared to a comparable delivery vehicle or liposomal structure without a modification or a delivery vehicle or liposomal structure with a modification comprising PEG. In some cases, a modification to a delivery vehicle occurs via a bond. A bond can be covalent, noncovalent, polar, ionic, hydrogen, or any combination thereof. A bond can be considered an association of two groups or portions of groups. For example, a delivery vehicle can be bonded to a PEG via a linker comprising a covalent bond. In some cases, a bond can occur between two adjacent groups. Bonds can be dynamic. A dynamic bond can occur when one group temporarily associates with a second group. For example, a polynucleic acid in suspension within a liposome may bond with portions of a lipid bilayer during its suspension.

In some cases, a surface modification to the delivery vehicles herein can be a polyethylene glycol (PEG) addition. Methods of modifying liposomal surfaces with PEG can include its physical adsorption onto a liposomal surface, its covalent attachment onto liposomes, its coating onto a liposome, or any combination thereof. In some cases, PEG can be covalently attached to a lipid particle before a liposome can be formed.

A variety of molecular weights of PEG may be used. PEG can range from about 10 to about 100 units of an ethylene PEG component which may be conjugated to phospholipid through an amine group comprising or comprising about 1% to about 20%, preferably about 5% to about 15%, about 10% by weight of the lipids which are included in a lipid bilayer.

In certain cases, a nanostructure can further comprise at least one targeting agent. The term targeting agent can refer to a moiety, compound, antibody, etc. that specifically binds a particular type or category of cell and/or other particular type compounds, (e.g., a moiety that targets a specific cell or type of cell). A targeting agent can be specific (e.g., have an affinity) for the surface of certain target cells, a target cell surface antigen, a target cell receptor, or a combination thereof. In some cases, a targeting agent can refer to an agent that has a particular action (e.g., cleaves) when exposed to a particular type or category of substances and/or cells, and this action can drive the nanostructure to target a particular type or category of cell. Thus, the term targeting agent can refer to an agent that can be part of a nanostructure and plays a role in the nanostructure's targeting mechanism, although the agent itself may or may not be specific for the particular type or category of cell itself. In certain instances, the efficiency of the cellular uptake of a polynucleic acid delivered by a nanostructure can be enhanced and/or made more specific by incorporation of targeting agents into the present nanostructures. In certain embodiments, nanostructures described herein can comprise one or more small molecule targeting agents (e.g., carbohydrate moieties). Suitable targeting agents also include, by way of non-limiting example, antibodies, antibody-like molecules, or peptides, such as an integrin-binding peptides such as RGD-containing peptides, or small molecules, such as vitamins, e.g., folate, sugars such as lactose and galactose, or other small molecules. Cell surface antigens include a cell surface molecule such as a protein, sugar, lipid or other antigen on the cell surface. In specific embodiments, the cell surface antigen undergoes internalization. Examples of cell surface antigens targeted by the targeting agents of embodiments of the present nanoparticles include, but are not limited, to the transferrin receptor type 1 and 2, the EGF receptor, HER2/Neu, VEGF receptors, integrins, NGF, CD2, CD3, CD4, CDS, CDI9, CD20, CD22, CD33, CD43, i1)38. CD56, CD69, and the leucine-rich repeat-containing G-protein coupled receptor 5 (LGR5). A targeting agent can also comprise an artificial affinity molecule, e.g., a peptidomimetic or an aptamer. Peptidomimetics can refer to compounds in which at least a portion of a peptide, such as a therapeutic peptide, is modified, and the three-dimensional structure of the peptidomimetic remains substantially the same as that of the peptide. Peptidomimetics (both peptide and non-peptidyl analogues) may have improved properties (e.g., decreased proteolysis, increased retention or increased bioavailability). Peptidomimetics generally have improved oral availability, which makes them especially suited to treatment of disorders in a human or animal. It should be noted that peptidomimetics may or may not have similar two-dimensional chemical structures, but share common three-dimensional structural features and geometry.

In some embodiments, the targeting agent can be a proteinaceous targeting agent (e.g., a peptide, and antibody, an antibody fragment). In some specific embodiments, a nanostructure can comprise a plurality of different targeting agents. In the embodiments herein, the compositions and methods include an MPP which provides mucus-penetration ability to the compositions and can also provide cell penetration. In some embodiments, the MPP can act also as a targeting agent. In other embodiments, a targeting agent is included in the composition in addition to an MPP.

In some embodiments, one or more targeting agents (which can be an MPP, a separate targeting agent or a combination of an MPP and a separate targeting agent) can be coupled to the polymers that form the nanostructure. In some cases, the targeting agents can be bound to a polymer that coats a nanostructure. In some instances, a targeting agent can be covalently coupled to a polymer. In some cases, a targeting agent can be bound to a polymer such that a targeting agent can be substantially at or near the surface of the resulting nanostructure. In certain embodiments, a monomer comprising a targeting agent residue (e.g, a polymerizable derivative of a targeting agent such as an (alkyl) acrylic acid derivative of a peptide) can be co-polymerized to form the copolymer forming the nanostructure provided herein. In certain embodiments, one or more targeting agents can be coupled to the polymer of the present nanoparticles through a linking moiety. In some embodiments, the linking moiety coupling the targeting agent to the membrane-destabilizing polymer can be a cleavable linking moiety (e.g., comprises a cleavable bond). In some embodiments, the linking moiety can be cleavable and/or comprises a bond that can be cleavable in endosomal conditions. In some embodiments, the linking moiety can be cleavable and/or comprise a bond that can be cleaved by a specific enzyme (e.g., a phosphatase, or a protease). In some embodiments, the linking moiety can be cleavable and/or comprise a bond that may be cleavable upon a change in an intracellular parameter (e.g., pH, redox potential), in some embodiments, a linking moiety can be cleavable and/or comprise a bond that can be cleaved upon exposure to a matrix metalloproteinase (MMP) (e.g., MMP-cleavable peptide linking moiety).

In certain cases, a targeting mechanism of a nanoparticle can depend on a cleavage of a cleavable segment in a polymer. For instance, the present polymers can comprise a cleavable segment that, when cleaved, exposes the nanoparticle and/or the core of a nanoparticle. The cleavable segment can be located at either or both terminal ends of the present polymers in some embodiments. In some embodiments the cleavable segment is located along a length of a polymer, and optionally can be located between blocks of a polymer. For example, in certain embodiments the cleavable segment can be located between a first block and a second block of a polymer, and when a nanoparticle can be exposed to a particular cleaving substance the first block can be cleaved from a second block. In specific embodiments a cleavable segment can be an MMP-cleavable peptide that can be cleaved upon exposure to MMP.

Attachment of a targeting agent, such as an antibody, to a polymer can be achieved in any suitable manner, e.g., by any one of a number of conjugation chemistry approaches including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl tinkers, sulfhydryl-sulfhydryl linkers, carbohydrate-hydroxyl linkers, carbohydrate-carbohydrate linkers, and hydroxyl-hydroxyl linkers. In specific embodiments, “click” chemistry can be used to attach the targeting agent to the polymers of the nanoparticles provided herein. A large variety of conjugation chemistries are optionally utilized, in some embodiments, targeting agents can be attached to a monomer and the resulting compound can then be used in a polymerization synthesis of a polymer (e.g., copolymer) utilized in a nanoparticle described herein. In some embodiments, a targeting agent can be attached to the sense or antisense strand of siRNA bound to a polymer of a nanoparticle. In certain embodiments, a targeting agent can be attached to a 5′ or a 3′ end of the sense or the antisense strand.

Methods for linking compounds can include but are not limited to proteins, labels, and other chemical entities, to nucleotides. Cross-linking reagents such as n-maleimidobutyryloxy-succinimide ester (GMBS) and sulfo-GMBS, have reduced immunogenicity. Substituents have been attached to the 5′ end of preconstructed oligonucleotides using amidite or H-phosphonate chemistry. Substituents can also be attached to the 3′ end of oligomers. This last method utilizes 2,2′-dithioethanol attached to a solid support to displace diisopropylamine from a 3′ phosphonate bearing the acridine moiety and is subsequently deleted after oxidation of the phosphorus. Alternatively, an oligonucleotide may include one or more modified nucleotides having a group attached via a linker arm to the base. For example, the attachment of biotin to the C-5 position of dUTP by an allylamine linker arm may be utilized. The attachment of biotin and other groups to the 5-position of pyrimidines via a linker arm may also be performed.

Chemical cross-linking may include the use of spacer arms, i.e., linkers or tethers. Spacer arms provide intramolecular flexibility or adjust intramolecular distances between conjugated moieties and thereby may help preserve biological activity. A spacer arm may be in the form of a peptide moiety comprising spacer amino acids. Alternatively, a spacer arm may be part of the cross-linking reagent, such as in “long-chain SPDP”.

A variety of coupling or crosslinking agents such as protein A, carbodiimide, dimaleimide, dithio-bis-nitrobenzoic acid (DTNB), N-succinimidyl-5-acetyl-thioacetate (SATA), and N-succinimidyl-3-(2-pyrid-yldithio)propionate (SPDP), 6-hydrazinonicotimide (HYNIC), N3S and N2S2 can be used in well-known procedures to synthesize targeted constructs. For example, biotin can be conjugated to an oligonucleotide via DTPA using a bicyclic anhydride method. In addition, sulfosuccinimidyl 6-(biotinamido)hexanoate (NHS-LC-biotin, which can be purchased from Pierce Chemical Co. Rockford, Ill.), “biocytin,” a lysine conjugate of biotin, can be useful for making biotin compounds due to the availability of a primary amine. In addition, corresponding biotin acid chloride or acid precursors can be coupled with an amino derivative of the therapeutic agent by known methods. By coupling a biotin moiety to the surface of a particle, another moiety may be coupled to avidin and then coupled to the particle by the strong avidin-biotin affinity, or vice versa. In certain embodiments where a polymeric particle comprises PEG moieties on the surface of the particle, the free hydroxyl group of PEG may be used for linkage or attachment (e.g., covalent attachment) of additional molecules or moieties to the particle.

In embodiments, a liposome modification can provide biocompatibility and can be modified to possess targeting species including, for example, targeting peptides including antibodies, aptamers, polyethylene, or combinations thereof. A targeting species can also be a receptor. In some cases, a T cell receptor (TCR), B cell receptor (BCR), single chain variable fragment (scFv), chimeric antigen receptor (CAR), or combinations thereof are used.

### Mucus-Penetrating Particles and Particle Treatments

Mucus-penetrating particle as used herein, can refer to particles which have been coated with a mucosal penetration enhancing coating. In some cases, a particle can be or can deliver a particle of an active agent, such as a therapeutic, diagnostic, prophylactic, and/or nutraceutical agent (i.e., drug particle) that can be coated with a mucosal penetrating enhancing coating. In other cases, particles can be formed of a matrix material, such as a polymeric material, in which a therapeutic, diagnostic, prophylactic, and/or nutraceutical agent can be encapsulated, dispersed, and/or associated. Coating material can be covalently or non-covalently associated with a drug particle or polymeric particle II.

Further, provided herein can be a delivery vehicle that can pass through a mucosal barrier at a greater rate than other delivery vehicle, e.g., unmodified delivery vehicle. A delivery vehicle may pass through a mucosal barrier at a rate that is at least 2, 5, 10, 20, 30, 50, 100, 200, 500, 1000- or greater fold higher than, e.g., an unmodified delivery vehicle of a similar size. In some cases, a non-PEG modified delivery vehicle can penetrate a mucosal barrier more efficiently than a PEG-modified delivery vehicle as measured by a transwell migration assay.

The delivery vehicles for use with the compositions and methods herein can contain polymers. A polymer can be any polymeric particle. Any number of biocompatible polymers can be used to prepare delivery vehicles such as nanoparticles. In one embodiment, a biocompatible polymer can be biodegradable. In another embodiment, a particle may not be non-degradable. In other embodiments, particles can be a mixture of degradable and non-degradable particles.

The delivery vehicles of the compositions and methods herein can have a near-neutral zeta potential from about −100 mV to about 100 mV. An MPP can have a zeta potential from about −50 mV to about 50 mV, from about −30 mV to about 30 mV, from about −20 mV to about 20 mV, from about −10 mV to about 10 mV, from about −5 mV to about 5 mV.

Biodegradable polymers typically differ from non-biodegradable polymers in that the former may degrade during use. In certain embodiments, such use involves in vivo use, such as in vivo therapy, and in other certain embodiments, such use involves in vitro use. In general, degradation attributable to biodegradability involves the degradation of a biodegradable polymer into its component subunits, or digestion, e.g., by a biochemical process, of the polymer into smaller, non-polymeric subunits. In certain embodiments, two different types of biodegradation may generally be identified. For example, one type of biodegradation may involve cleavage of bonds (whether covalent or otherwise) in the polymer backbone. In such biodegradation, monomers and oligomers typically result, and even more typically, such biodegradation occurs by cleavage of a bond connecting one or more of subunits of a polymer. In contrast, another type of biodegradation may involve cleavage of a bond (whether covalent or otherwise) internal to sidechain or that connects a side chain to the polymer backbone. For example, a therapeutic agent or other chemical moiety attached as a side chain to the polymer backbone may be released by biodegradation. In certain embodiments, one or the other or both general types of biodegradation may occur during use of a polymer. The degradation rate of a biodegradable polymer often depends in part on a variety of factors, including the chemical identity of the linkage responsible for any degradation, the molecular weight, crystallinity, biostability, and degree of cross-linking of such polymer, the physical characteristics (e.g., shape and size) of the implant, and the mode and location of administration. For example, the greater the molecular weight, the higher the degree of crystallinity, and/or the greater the biostability, the biodegradation of any biodegradable polymer is usually slower.

In certain embodiments a biodegradable polymer may also have a therapeutic agent or other material associated with it, the biodegradation rate of such polymer may be characterized by a release rate of such materials. For example, the biodegradation rate may depend on not only the chemical identity and physical characteristics of the polymer, but also on the identity of material(s) incorporated therein. In some cases, polymeric formulations of the present invention biodegrade within a period that is acceptable in a desired application. In certain embodiments, such as in vivo therapy, such degradation occurs in a period usually less than about five years, one year, six months, three months, one month, fifteen days, five days, three days, or even one day or less (e.g., 4-8 hours) on exposure to a physiological solution with a pH between 6 and 8 having a temperature of between 25 and 37° C. In other embodiments, the polymer degrades in a period of between about one hour and several weeks, depending on the desired application.

Polymers for use with the compositions and methods herein are those such as provided in PCT/US17/61111, which is incorporated by reference herein in its entirety.

In some cases, a delivery vehicle containing a cargo such as a therapeutic, diagnostic, prophylactic, and/or nutraceutical agent can be coated with a mucosal penetration enhancing coating. A delivery vehicle can be a microparticle or a nanoparticle. A coating can be applied using any means, techniques, supplies, or combinations thereof. A mucosal penetration enhancing coating can be covalently or non-covalently associated with a lipid, polymer, or any combination. In some embodiments, it may be non-covalently associated. In other embodiments, a lipid or polymer can contain a reactive functional group or one can be incorporated to which a mucosal penetration enhancing coating can be covalently bound.

Nanoparticles may be coated with or contain one or more surface altering agents. In some cases, a surface-alternating agent can provide a direct therapeutic effect, such as reducing inflammation. A nanoparticle can be coated such as a coating provides a nanoparticle with a near-neutral zeta potential. A coating can be PEGylation. A coating can be a partial coating or a full coating. Examples of surface-altering agents include, but are not limited to, proteins, including anionic proteins (e.g., albumin), surfactants, sugars or sugar derivatives (e.g., cyclodextrin), therapeutics agents, and polymers. Polymers may also include heparin, polyethylene glycol (“PEG”) and poloxomers (polyethylene oxide block copolymers). A polymer may be PEG, PLURONIC F127®, PEG2000, or any derivative, modified version thereof, or combination thereof.

A surface-altering agent may increase charge or hydrophilicity of the delivery vehicle or liposomal particle, or otherwise decrease interactions between the particle and mucus, thereby promoting motility through mucus. A surface-altering agent may enhance the average rate at which the polymeric or liposomal particles, or a fraction of the particles, move in or through mucus. Examples of suitable surface-altering agents include but are not limited to anionic protein (e.g., serum albumin), nucleic acids, surfactants such as cationic surfactants (e.g., dimethyldioctadecyl-ammonium bromide), sugars or sugar derivatives (e.g., cyclodextrin), polyethylene glycol, mucolytic agents, or other non-mucoadhesive agents. Certain agents, e.g., cyclodextrin, may form inclusion complexes with other molecules and can be used to form attachments to additional moieties and facilitate the functionalization of the particle surface and/or the attached molecules or moieties. In some cases, a surface altering agent can cause a surface modification. A surface altering agent can be PEG, PEG can be a polymer used in a delivery vehicle. A surface modification can be interchanged with modification. In some cases, a modification can refer to a surface modification. In other cases, a modification may not refer to a surface modification.

In some cases, mucus disruptive agents can be delivered or can be found on a particle. Mucus can be a biological gel that coats tissue surfaces generally exposed to the external environment such as the airways, GI tract, eyes and reproductive tract. It can form a defensive barrier that captures or blocks foreign bodies and pathogenic bacteria from reaching the underlying cells and causing damage or disease. Mucus is predominantly comprised of water (around 95%), glycoproteins (2-5%), lipids, and salts. Glycosylated proteins can be from a MUC family. In some routes of drug administration, such as oral, nasal, pulmonary or vaginal, mucus may act as a barrier. delivery vehicle carrying a polynucleic acid or other cargo may need to be specifically designed to penetrate a mucosal layer before they are removed via mucus clearance. Enhancing mucosal penetration and permeation is therefore essential to avoid capture and excretion from a mucosal barrier, and to fully exploit the benefits of nanoparticle-based drug delivery.

Mucus disruptive agents can be an NSAID, a miRNA against B-catenin or an agent that may be known to disrupt mucus. Mucus disruptive agents can be surface altering agents. In some cases, disrupting mucous can be eliminating production of mucous. In other cases, disrupting mucous can be reducing the production of mucous. For example, reducing mucous may mean reducing the production of mucous by targeting a cell that generates mucous. Mucous disruption may also mean adjusting the consistency of mucous. For example, mucous disruption may mean loosening the consistency of mucous.

In some cases, a nanoparticle can be coated with or contain polyethylene glycol (PEG). Alternatively, a PEG can be in the form of blocks covalently bound (e.g., in the interior or at one or both terminals) to a lipid used to form a nanoparticles. In particular embodiments, a nanoparticle can be formed from block copolymers containing PEG. A nanoparticle can also be prepared from block copolymers containing PEG, wherein PEG may be covalently bound to a terminal of a base lipid. Representative PEG molecular weights can include 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, 500 kDa, and 1 MDa and all values within the range of 300 Daltons to 1 MDa. A PEG can be about 2 kDa in some cases. PEG of any given molecular weight may vary in other characteristics such as length, density, and branching.

A PEG coating can be applied at any concentration. In some cases, a concentration between lipid to PEG can be 5 to 10%. A concentration can be at least 5% or at most 10%. In some cases, a concentration can be over 10%. A concentration can be or can be about 1%, 2%, 3%, 4%, 5%, 6%, 7%, 8%, 9%, 10%, or over 10%. In some embodiments, PEG surface density can be controlled by preparing a nanoparticle from a mixture of PEGylated and non-PEGylated particles. For example, a surface density of PEG on nanoparticles can be precisely controlled by preparing particles from a mixture of poly (lactic-co-glycolic acid) and poly (ethylene glycol) (PLGA-PEG).

In some cases, a PEG coating can be measured for density on a nanoparticle. Quantitative 1H nuclear magnetic resonance (NMR) can be used to measure surface PEG density on nanoparticles. In some cases, a density can be or can be about 10 to 16 PEG chains/100 nm2. In some cases a density can be over 10 to 16 PEG chains/100 nm2. This density threshold may vary depending on a variety of factors including a liposome of a nanoparticle, particle size, and/or molecular weight of PEG. Density of a coating that can be applied to a liposome can be varied based on a variety of factors including a surface altering material and a composition of a particle. In one embodiment, density of a surface altering material, such as PEG, as measured by ¹H NMR can be or can be about, 0.1, 0.2, 0.5, 0.8, 1, 2, 5, 8, 10, 15, 20, 25, 40, 50, 60, 75, 80, 90, or 100 chains per nm2. The range above can be inclusive of all values from 0.1 to 100 units per nm2. In some cases, a density of a surface altering material, such as PEG, can be or can be about 1 to about 25 chains/nm2, can be or can be about 1 to about 20 chains/nm2, can be or can be about 5 to about 20 chains/nm2, can be or can be about 5 to about 18 chains/nm2, can be or can be about 5 to about 15 chains/nm2, or can be or can be about 10 to about 15 chains/nm2. In other cases a density can be or can be about 0.05 to about 0.5 PEG chains/nm2. PEG can be 10 to 20 chains per 100 nm2.

A concentration of a surface altering material, such as PEG, can also be varied. In particular embodiments, a density of a surface-altering material (e.g., PEG) can be such that a surface-altering material (e.g. PEG) adopted an extended brush configuration. In other embodiments, a mass of a surface-altering moiety can be at least or can be at least about 1/10,000, 1/7500, 1/5000, 1/4000, 1/3400, 1/2500, 1/2000, 1/1500, 1/1000, 1/750, 1/500, 1/250, 1/200, 1/150, 1/100, 1/75, 1/50, 1/25, 1/20, 1/5, 1/2, or 9/10 of a mass of a nanoparticle. The range above can be inclusive of all values from 1/10,000 to 9/10.

A polymer such as PEG or POZ, can be at a density from about 0.05 μg/nm 2 to about 0.25 μg/nm2. A polymer can also be at a density from about 0.01 μg/nm 2, 0.02 μg/nm 2, 0.03 μg/nm 2, 0.04 μg/nm 2, 0.05 μg/nm 2, 0.06 μg/nm 2, 0.07 μg/nm 2, 0.08 μg/nm 2, 0.09 μg/nm 2, 0.1 μg/nm 2, 0.15 μg/nm 2, 0.2 μg/nm 2, 0.25 μg/nm 2, 0.3 μg/nm 2, 0.35 μg/nm 2, 0.4 μg/nm 2, 0.45 μg/nm 2, 0.5 μg/nm 2, 0.55 μg/nm 2, 0.6 μg/nm 2, 0.65 μg/nm 2, 0.7 μg/nm 2, 0.75 μg/nm 2, 0.8 μg/nm 2 μg/nm 2, 0.85 μg/nm 2, 0.9 μg/nm 2, 0.95 μg/nm 2, or up to 1 μg/nm 2. In some embodiments μg/nm 2 with regard to density can refer to μg polymer per nm 2 delivery vehicle or liposomal structure surface. In some embodiments μg refers to microgram. In some embodiments, nm refers to nanometer.

In some cases, a polymer can be a poly (2-alkyl-2-oxazoline) addition. Similar to PEG, poly (2-alkyl-2-oxazoline) has “stealth” properties, is non-toxic and biocompatible, has a pendent group for further functionalization, and a high degree of renal clearance with low bioaccumulation. Poly (2-alkyl-2-oxazoline) can increase mucosal penetration of a structure. In some cases, non-PEG coated structures may have increased mucosal penetration to structures coated with PEG. Increased mucosal penetration can be measured by a transwell migration assay. Additional assays that can be utilized to measure mucosal penetration can comprise multiple particle tracking (MPT), Ussing chamber, or a combination thereof. In some cases, a mucosal penetration assay can record a delivery vehicle's dynamic transit in a mucus using fluorescence microscopy, such as fluorescence recovery after photobleaching (FRAP) and multiple particle tracking (MPT). FRAP can be the fluorescently labeled delivery vehicle's exposure to a laser beam to form a floating white spot. The diffusion coefficient can be obtained by recovery of a fluorescence intensity, which may result following diffusion of a fluorescently labeled molecule into an area with a flow of delivery vehicle.

To better understand a fate of a particles and how results might translate in humans, a mucosal penetration study can adopt an animal model to investigate a therapeutic effect or pharmacokinetics of a delivery vehicle, which mainly include isolated intestinal experiments, in situ experiments and in vivo experiments. For example, in an in situ experiment of mucosal penetration a portion of a small intestine can be excised from an abdominal cavity, subsequently ligated at both ends to make an isolated “loop”, and a delivery vehicle can be directly injected into a loop. After a chosen time period, an animal can be sacrificed and the intestinal loop can be removed from a body cavity for further morphology or quantitative analysis.

In some cases a coating can be an enteric coating. Enteric coatings can be utilized to prevent or minimize dissolution in the stomach but allow dissolution in the small intestine. In some embodiments, a coating can include an enteric coating. An enteric coating can be a barrier applied to oral medication that prevents release of medication before it reaches the small intestine. Delayed-release formulations, such as enteric coatings, can an irritant effect on the stomach from administration of a medicament from dissolving in the stomach. Such coatings are also used to protect acid-unstable drugs from the stomach's acidic exposure, delivering them instead to a basic pH environment (intestine's pH 5.5 and above) where they may not degrade.

Dissolution can occur in an organ. For example, dissolution can occur within a duodenum, jejunum, ilium, and/or colon, or any combination thereof. In some cases, dissolution can occur in proximity to a duodenum, jejunum, ilium, and/or colon. Some enteric coatings work by presenting a surface that is stable at a highly acidic pH found in the stomach, but break down rapidly at a less acidic (relatively more basic) pH. Therefore, an enteric coated pill may not dissolve in the acidic environment of the stomach, but can dissolve in an alkaline environment present in a small intestine. Examples of enteric coating materials include, but are not limited to, methyl acrylate-methacrylic acid copolymers, cellulose acetate succinate, hydroxy propyl methyl cellulose phthalate, hydroxy propyl methyl cellulose acetate succinate (hypromellose acetate succinate), polyvinyl acetate phthalate (PVAP), methyl methacrylate-methacrylic acid copolymers, sodium alginate and stearic acid.

An enteric coating can be applied at a functional concentration. An enteric coating can be cellulose acetate phthalate, Polyvinyl acetate phthalate, Hydroxypropylmethylcellulose acetate succinate, Poly(methacylic acid-co-ethyl acrylate) 1:1, Poly(methacrylic acid-co-ethyl acrylate) 1:1, Poly(methacylic acid-co-methyl methacrylate) 1:1, Poly(methacylic acid-co-methyl methacrylate) 1:1, Poly(methacylic acid-co-methyl methacrylate) 1:2, Poly(methacylic acid-co-methyl methacrylate) 1:2, Poly(methyl acrylate-co-methyl methacrylate-co-methacrylic acid) 7:3:1, or any combination thereof. An enteric coating can be applied from about 6 mg/(cm2) to about 12 mg/(cm2). An enteric coating can also be applied to a structure from about 1 mg/(cm2), 2 mg/(cm2), 3 mg/(cm2), 4 mg/(cm2), 5 mg/(cm2), 6 mg/(cm2), 7 mg/(cm2), 8 mg/(cm2), 9 mg/(cm2), 10 mg/(cm2), 11 mg/(cm2), 12 mg/(cm2), 13 mg/(cm2), 14 mg/(cm2), 15 mg/(cm2), 16 mg/(cm2), 17 mg/(cm2), 18 mg/(cm2), 19 mg/(cm2), to about 20 mg/(cm2).

In some embodiments, a pharmaceutical composition can be orally administered from a variety of drug formulations designed to provide delayed-release. Delayed oral dosage forms include, for example, tablets, capsules, caplets, and may also comprise a plurality of granules, beads, powders or pellets that may or may not be encapsulated. Tablets and capsules can represent oral dosage forms, in which case solid pharmaceutical carriers can be employed. In a delayed-release formulation, one or more barrier coatings may be applied to pellets, tablets, or capsules to facilitate slow dissolution and concomitant release of drugs into the intestine. Typically, a barrier coating can contain one or more polymers encasing, surrounding, or forming a layer, or membrane around a therapeutic composition or active core. In some embodiments, active agents, such as a polynucleic acid, can be delivered in a formulation to provide delayed-release at a pre-determined time following administration. The delay may be up to about 10 minutes, about 20 minutes, about 30 minutes, about 1 hour, about 2 hours, about 3 hours, about 4 hours, about 5 hours, about 6 hours, or up to 1 week in length. In some cases, an enteric coating may not be used to coat a particle.

Polymers or coatings that can be used to achieve enteric release can be anionic polymethacrylates (copoly-merisate of methacrylic acid and either methyl-methacrylate or ethylacrylate (Eudragit®), cellulose based polymers, e.g. cellulose acetatephthalate (Aquateric®) or polyvinyl derivatives, e.g. polyvinyl acetate phthalate (Coateric®) in some cases.

Depending upon the ratio of polynucleic acid to polymer and the nature of the particular polymer employed, the rate of polynucleic acid, such as minicircle DNA, release can be controlled. In some cases, a depot injectable formulation can be prepared by entrapping a polynucleic acid in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

A nanoparticle may have a variety of shapes and cross-sectional geometries that may depend, in part, upon the process used to produce it. In one case, a nanoparticle may have a shape that can be a sphere, a rod, a tube, a flake, a fiber, a plate, a wire, a cube, or a whisker. A nanoparticle may include particles having two or more of the aforementioned shapes. In another case, a cross-sectional geometry of the particle may be one or more of circular, ellipsoidal, triangular, rectangular, or polygonal. In one embodiment, a nanoparticle may be a non-spherical particle. For example, a nanoparticle may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis can be substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

Cargo for Deliver Vehicle

Provided herein are delivery vehicles with mucus-penetrating features (including with an MPP) that include a cargo. In some cases, a cargo can be, for example, a nucleic acid, a dye, drug, protein, a nanoparticle, or chemical agent. Cargo can include, for example, a chemical compound, therapeutic agent, small molecule drug, biologic drug, peptide, polypeptide, protein, antibody, polynucleotide, oligonucleotide, DNA, double stranded DNA, single stranded DNA, minicircle DNA, double stranded RNA, single stranded RNA, RNAs (including shRNA and siRNA), nucleic acid vector for expression of RNA and protein, dye, fluorescent dye, polysaccharide, saccharide, lipid, peptidomimetic, or a combination thereof. The cargo may have a therapeutic function, a diagnostic function, a localization or tagging function. The cargo may act in concert with other molecules present at or delivered to the cells and tissue of interest.

In some embodiments, the cargo can be a nucleic acid. A nucleic acid can be a vector. Nucleic acid can be DNA- or RNA-based. DNA-based vectors can be non-viral, and include molecules such as plasmids, minicircles, closed linear DNA (doggybone), linear DNA, and single-stranded DNA. A nucleic acid that can be present in a lipid-nucleic acid particle includes any form of nucleic acid that is known. The nucleic acids used herein can be single-stranded DNA or RNA, or double-stranded DNA or RNA, or DNA-RNA hybrids. Examples of double-stranded DNA include structural genes, genes including control and termination regions, and self-replicating systems such as viral or plasmid DNA. Examples of double-stranded RNA include siRNA and other RNA interference reagents. Single-stranded nucleic acids include antisense oligonucleotides, ribozymes, microRNA, and triplex-forming oligonucleotides. The nucleic acid that is present in a lipid-nucleic acid particle may include one or more of the oligonucleotide modifications described below. Nucleic acids may be of various lengths, generally dependent upon the particular form of nucleic acid. For example, in particular embodiments, plasmids or genes may be from about 1,000 to 100,000 nucleotide residues in length. In particular embodiments, oligonucleotides may range from about 10 to 100 nucleotides in length. In various related embodiments, oligonucleotides, single-stranded, double-stranded, and triple-stranded, may range in length from about 10 to about 50 nucleotides, from about 20 to about 50 nucleotides, from about 15 to about 30 nucleotides, from about 20 to about 30 nucleotides in length. In particular embodiments, oligonucleotides may range from about 2 nucleotides to 10 nucleotides in length.

DNA-based vectors can also be viral, and include adeno-associated virus, lentivirus, adenovirus, and others. Vectors can also be RNA. RNA vectors can be linear or circular forms of unmodified RNA. They can also include various nucleotide modifications designed to increase half-life, decrease immunogenicity, and/or increase level of translation. A vector as used herein can be composed of either DNA or RNA. In some embodiments, a vector can be composed of DNA. Vectors can be capable of autonomous replication in a prokaryote such as E. coli, used for growth. In some embodiments a vector may be stably integrated into a genome of an organism. In other cases, a vector can remain separate, either in a cytoplasm or a nucleus. In some embodiments, a vector can contain a targeting sequence. In some embodiments, a vector can contain an antibiotic resistance gene. A vector can contain regulatory elements for regulating gene expression. In some cases, a mini-circle can be enclosed within a liposome.

A cargo can be a gene, high molecular weight DNA, plasmid DNA, an antisense oligonucleotide, peptides, peptidomimetics, ribozymes, peptide nucleic acids, a chemical agent such as a chemotherapeutic molecule, or any large molecule including, but not limited to, DNA, RNA, viral particles, growth factors cytokines, immunomodulating agents and other proteins, including proteins which when expressed present an antigen which stimulates or suppresses the immune system.

Cargo can include, for example, small molecule drugs, peptides, proteins, antibodies, DNA (minicircle DNA for example), double stranded DNA, single stranded DNA, double stranded RNA, single stranded RNA, RNAs (including shRNA and siRNA (which may also be expressed by the plasmid DNA incorporated as cargo within a liposome), antiviral agents such as acyclovir, zidovudine and the interferons; antibacterial agents such as aminoglycosides, cephalosporins and tetracyclines; antifungal agents such as polyene antibiotics, imidazoles and triazoles; antimetabolic agents such as folic acid, and purine and pyrimidine analogs; antineoplastic agents such as the anthracycline antibiotics and plant alkaloids; sterols such as cholesterol; carbohydrates, e.g., sugars and starches; amino acids, peptides, proteins such as cell receptor proteins, immunoglobulins, enzymes, hormones, neurotransmitters and glycoproteins; radiolabels such as radioisotopes and radioisotope-labeled compounds; radiopaque compounds; fluorescent compounds; mydriatic compounds; bronchodilators; local anesthetics; dyes, fluorescent dyes, including fluorescent dye peptides which may be expressed by a DNA incorporated within a liposome, or any combination thereof.

In some cases, the cargo can be a portion of a gene that can be expressed by a nucleic acid. A portion of a gene can be from three nucleotides up to the entire whole genomic sequence. For example, a portion of a gene can be from about 1% up to about 100% of an endogenous genomic sequence. A portion of a gene can be from about 1%, 10%, 20%, 30%, 40%, 50%, 60%, 70%, 80%, 90%, or up to about 100% of a whole genomic sequence of a gene.

Minicircle (MC) DNA can be similar to plasmid DNA as both may contain expression cassettes that may permit transgene products to be made at high levels shortly after delivery. In some cases, a MC can differ in that MC DNA can be devoid of prokaryotic sequence elements (e.g., bacterial origin of replication and antibiotic-resistance genes). Removal of prokaryotic sequence elements from a backbone plasmid DNA can be achieved via site-specific recombination in Escherichia coli before episomal DNA isolation. The lack of prokaryotic sequence elements may reduce MC size relative to its parental full-length (FL) plasmid DNA, which may lead to enhanced transfection efficiencies. The result may be that when compared with their FL plasmid DNA counterparts, MCs can transfect more cells and may permit sustained high level transgene expression upon delivery.

In some cases, a minicircle DNA can be free of a bacterial origin of replication. For example, a minicircle DNA or closed linear DNA, can be free of a bacterial origin of replication from about 50% of a bacterial origin of replication sequence or up to 100% of a bacterial origin of replication. In some cases, a bacterial origin of replication is truncated or inactive. A polynucleic acid can be derived from a vector that initially encoded a bacterial origin of replication. A method can be utilized to remove the entirety of a bacterial origin of replication or a portion thereof, leaving a polynucleic acid free of a bacterial origin of replication. In some cases, a bacterial origin of replication can be identified by its high adenine and thymine content.

Minicircle DNA vectors can be supercoiled minimal expression cassettes, derived from conventional plasmid DNA by site-specific recombination in vivo in Escherichia coli for the use in non-viral gene therapy and vaccination. Minicircle DNA may lack or have reduced bacterial backbone sequences such as an antibiotic resistance gene, an origin of replication, and/or inflammatory sequences intrinsic to bacterial DNA. In addition to their improved safety profile, minicircles can greatly increase efficiency of transgene expression.

In some cases, a nucleic acid can encode for a heterologous sequence. A heterologous sequence can provide for subcellular localization (e.g., a nuclear localization signal (NLS) for targeting to a nucleus; a mitochondrial localization signal for targeting to a mitochondria; a chloroplast localization signal for targeting to a chloroplast; an ER retention signal; and the like). In some case, a polynucleic acid, such as minicircle DNA or closed linear DNA, can comprise a nuclear localization sequence (NLS).

In some embodiments, a vector encodes a protein such as APC. A vector can comprise one or more nuclear localization sequences (NLSs). A number of NLS sequences can be from about 1, 2, 3, 4, 5, 6, 7, 8, 9, 10, or more NLSs. In some embodiments, a vector comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxyl-terminus, or a combination of these (e.g. one or more NLS at the amino-terminus and one or more NLS at the carboxyl terminus). When more than one NLS is present, each may be selected independently of the others, such that a single NLS may be present in more than one copy and/or in combination with one or more other NLSs present in one or more copies.

Non-limiting examples of NLSs can include an NLS sequence derived from: the NLS of the SV40 virus large T-antigen, having the amino acid sequence PKKKRKV; the NLS from nucleoplasmin (e.g. the nucleoplasmin bipartite NLS with the sequence KRPAATKKAGQAKKKK); the c-myc NLS having the amino acid sequence PAAKRVKLD or RQRRNELKRSP; the hRNPA1 M9 NLS having the sequence NQSSNFGPMKGGNFGGRSSGPYGGGGQYFAKPRNQGGY; the sequence RMRIZFKNKGKDTAELRRRRVEVSVELRKAKKDEQILKRRNV of the IBB domain from importin-alpha; the sequences VSRKRPRP and PPKKARED of the myoma T protein; the sequence POPKKKPL of human p53; the sequence SALIKKKKKMAP of mouse c-abl IV; the sequences DRLRR and PKQKKRK of the influenza virus NS1; the sequence RKLKKKIKKL of the Hepatitis virus delta antigen; the sequence REKKKFLKRR of the mouse M×1 protein; the sequence KRKGDEVDGVDEVAKKKSKK of the human poly(ADP-ribose) polymerase; and the sequence RKCLQAGMNLEARKTKK of the steroid hormone receptors (human) glucocorticoid. In general, the one or more NLSs can be of sufficient strength to drive accumulation of the minicircle DNA vector or short linear DNA vector in a detectable amount in the nucleus of a eukaryotic cell. A eukaryotic cell can be a human intestinal crypt cell.

Detection of accumulation in the nucleus may be performed by any suitable technique. For example, a detectable marker may be fused to a vector, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. An embodiment herein can exhibit time dependent pH triggered release of a liposome cargo into a target site. An embodiment herein can contain and provide cellular delivery of complex multiple cargoes. An additional cargo can be a small molecule, an antibody, an inhibitor such as a DNAse inhibitor or RNAse inhibitor.

In some cases, a particle may contain a DNAse inhibitor. A DNAse inhibitor may be localized within a particle or on a particle. In other cases, a polynucleic acid encoding for an inhibitor can be enclosed within a particle. In other cases, an inhibitor can be a DNA methyltransferase inhibitor such as DNA methyltransferase inhibitors-2 (DMI-2). DMI-2 can be produced by Streptomyces sp. strain No. 560. A structure of DMI-2 can be 4′″R,6aR,10S,10aS-8-acetyl-6a,10a-dihydroxy-2-methoxy-12-methyl-10-[4′-[3″-hydroxy-3″,5″-dimethyl-4″ (Z-2′″,4″′-dimethyl-2′″-heptenoyloxy) tetrahydropyran-1″-yloxy]-5′-methylcyclohexan-1′-yloxy]-1,4,6,7,9-pentaoxo-1,4,6,6a,7,8,9,10,10a,11-decahydronaphthacene. Other inhibitors, such as chloroquine, can also be enclosed within a particle or on a particle, such as on a surface of a particle.

Among the compositions and methods herein include compositions that have a cargo of nucleic acid that can be delivered to cells of the intestinal tract. For example, a polynucleic acid can be delivered by the mucus-penetrating compositions herein, such as delivery vehicles and delivery vehicles with an MPP to cells in the GI tract, such as an intestinal crypt stem cell. For example, a delivered polynucleic acid can be: (1) not normally found in intestinal epithelial stem cells; (2) normally found in intestinal epithelial stem cells, but not expressed at physiological significant levels; (3) normally found in intestinal epithelial stem cells and normally expressed at physiological desired levels in the stem cells or their progeny; (4) any other DNA which can be modified for expression in intestinal epithelial stem cells; and (5) any combination of the above. In some cases, the mucus-penetrating compositions herein can deliver a cargo, such as a nucleic acid, to cells of the GI tract and wherein a protein product encoded by the nucleic acid is secreted or otherwise transported to other cells and tissues.

A variety of protein and polypeptides can be delivered to cells of the GI tracts, such as an intestinal crypt stem cell, including proteins for treating metabolic disorders and endocrine disorders. Examples of proteins are phenylalanine hydroxylase, insulin, anti-diuretic hormone and growth hormone. Disorders include phenylketonuria, diabetes, organic acidurias, tyrosinemia, urea cycle disorders, familial hypercholesteremia. Genes for any of the proteins or peptides which can correct the defects in phenylketonuria, diabetes, organic acidurias, tyrosinemia, urea cycle disorders, familial hypercholesteremia can be introduced into stem cells such that the protein or peptide products are expressed by the intestinal epithelium. Coagulation factors such as antihemophilic factor (factor VIII), Christmas factor (factor IX) and factor VII can likewise be produced in the intestinal epithelium. Proteins which can be used to treat deficiency of a circulatory protein can also be expressed in the intestinal epithelium. Proteins which can be used to treat deficiency of a circulatory protein can be, for example, albumin for the treatment of an albuminemia, alpha-1-antitrypsin, hormone binding protein. Additionally, the intestinal symptoms of cystic fibrosis can be treated by inserting the gene for the normal cystic fibrosis transmembrane conductance regulator into the stem cells of intestinal epithelium. Abetalipoproteinemia can be treated by the insertion of the apolipoprotein B. Disaccharidase intolerance can be treated by the insertion of sucrase-isomaltose, lactase-phlorizin hydrolase and maltase-glucoamylase. The insertion of the intrinsic factor for the absorption of vitamin B12 or the receptor for the intrinsic factor/cobalamin complex for absorption of vitamin B12, as well as the transporter for bile acids can be inserted into the intestinal epithelium. Further, any drug which can be encoded by nucleic acid can be inserted into the stem cell of the intestinal epithelium to be secreted in localized, high concentrations for the treatment of cancer. In this respect, one skilled in the art will readily recognize that antisense RNA can be encoded into the stem cells after production of antisense it can incorporate into the cancerous cells for the treatment of cancer. Other examples for delivery include nucleic acids encoding proteins to treat congenital diarrhea diseases such as microvillus inclusion disease with Myo5B and inflammatory bowel disease with IL-10.

In some cases, a protein that is encoded by a nucleic acid comprised within delivery vehicle can be measured and quantified. In some cases, modified cells can be isolated and a western blot performed on modified cells to determine a presence and a relative amount of protein production as compared to unmodified cells. In other cases, intracellular staining of a protein utilizing flow cytometry can be performed to determine a presence and a relative amount of protein production. Additional assays can also be performed to determine if a protein, such as APC, is functional. For example, modified cells expressing an APC transgene, can be measured for cytosolic β-catenin expression and compared to unmodified cells. Reduced expression of β-catenin in the cytosol of modified cells as compared to unmodified cells can be indicative of a functional APC transgene. In other cases, a murine model of FAP can be utilized to determine functionality of a transgene encoding an APC protein. For example, mice with FAP can be treated with modified cells, encoding for APC, and a reduction of FAP disease measured versus untreated mice.

In certain embodiments, the compositions and methods herein include a cargo that can comprise an imaging agent that may be further attached to a detectable label (e.g., the label can be a radioisotope, fluorescent compound, enzyme or enzyme co-factor). The active moiety may be a radioactive agent, such as: radioactive heavy metals such as iron chelates, radioactive chelates of gadolinium or manganese, positron emitters of oxygen, nitrogen, iron, carbon, or gallium, 43K, 52Fe, 57Co, 67Cu, 67Ga, 68Ga, 123I, 125I, 131I, 132I, or 99Tc. A delivery vehicle including such a moiety may be used as an imaging agent and be administered in an amount effective for diagnostic use in a mammal such as a human. In this manner, the localization and accumulation of the imaging agent can be detected. The localization and accumulation of the imaging agent may be detected by radioscintiography, nuclear magnetic resonance imaging, computed tomography, or positron emission tomography. As will be evident to the skilled artisan, the amount of radioisotope to be administered is dependent upon the radioisotope. Those having ordinary skill in the art can readily formulate the amount of the imaging agent to be administered based upon the specific activity and energy of a given radionuclide used as the active moiety. Typically 0.1-100 millicuries per dose of imaging agent, 1-10 millicuries, and 2-5 millicuries can be administered. Thus, compositions useful as imaging agents can comprise a targeting moiety conjugated to a radioactive moiety that can comprise 0.1-100 millicuries, in some embodiments preferably 1-10 millicuries, in some embodiments preferably 2-5 millicuries, in some embodiments more preferably 1-5 millicuries. The means of detection used to detect the label is dependent of the nature of the label used and the nature of the biological sample used, and may also include fluorescence polarization, high performance liquid chromatography, antibody capture, gel electrophoresis, differential precipitation, organic extraction, size exclusion chromatography, fluorescence microscopy, or fluorescence activated cell sorting (FACS) assay. A targeting moiety can also refer to a protein, nucleic acid, nucleic acid analog, carbohydrate, or small molecule. The entity may be, for example, a therapeutic compound such as a small molecule, or a diagnostic entity such as a detectable label. A locale may be a tissue, a particular cell type, or a subcellular compartment. In one embodiment, the targeting moiety can direct the localization of an active entity. The active entity may be a small molecule, protein, polymer, or metal. The active entity, such as a liposome comprising a nucleic acid, may be useful for therapeutic, prophylactic, or diagnostic purposes. In some cases, a moiety may allow a delivery vehicle to penetrate a blood brain barrier.

In other cases, a computerized tomography scan (CT) can or magnetic resonance imaging (MRI) can be taken. A CT can be taken on a slice thickness of 5 mm or less. If CT scans have slice thickness greater than 5 mm, the minimum size for a measurable lesion should be twice the slice thickness. In some cases, an FDG-PET scan can be used. FDG-PET can be used to evaluate new lesions. A negative FDG-PET at baseline, with a positive FDG-PET at follow up is a sign of progressive disease (PD) based on a new lesion. No FDG-PET at baseline and a positive FDG-PET at follow up: if a positive FDG-PET at follow-up corresponds to a new site of disease confirmed by CT, this is PD. If a positive PDG-PET at follow up corresponds to a pre-existing site of disease on CT that may not be progressing on a basis of anatomic imagines, this may not be PD. In some cases, FDG-PET may be used to upgrade a response to a CR in a manner similar to biopsy in cases where a residual radiographic abnormality is thought to represent fibrosis or scarring. A positive FDG-PET scan lesion means one which is FDG avid with an uptake greater than twice that of the surrounding tissue on an attenuation corrected image.

In some cases an evaluation of a lesion can be performed. A complete response (CR) can be a disappearance of all target lesions. Any pathological lymph nodes (target or non-target) may have reduction in short axis to less than 10 mm. A partial response (PR) can be at least a 30% decrease in a sum of the diameters of target lesions, taking as reference the baseline sum of diameters. Progressive disease (PD) can be at least a 20% increase in the sum of the diameters of target lesions, taking as reference the smallest sum. In addition to the relative increase of 20%, the sum must also demonstrate an absolute increase of at least 5 mm. Stable disease (SD) can be neither sufficient shrinkage to quality for PR nor sufficient increase to quality for PD, taking as reference the smallest sum of diameters.

In some cases, non-target lesions can be evaluated. A complete response of a non-target lesion can be a disappearance and normalization of tumor marker level. All lymph nodes must be non-pathological in size (less than 10 mm short axis). If tumor markers are initially above the upper normal limit, they must normalize for a patient to be considered a complete clinical response. Non-CR/Non-PD is persistence of one or more non-target lesions and or maintenance of tumor marker level above the normal limit. Progressive disease can be appearance of one or more new lesions and or unequivocal progression of existing non-target lesions. Unequivocal progression should not normally trump target lesion status.

In some cases, a best overall response can be the best response recorded from the start of treatment until disease progression/recurrence.

### Pharmaceutical Compositions and Formulations

The compositions described throughout can be formulation into a pharmaceutical medicament and be used to treat a human or mammal, in need thereof, diagnosed with a disease or condition, particularly in tissues and cells that are associated with a layer of mucus through which the therapeutic agent must be delivered. Medicaments can be co-administered with any additional therapy.

A disease that can be treated with a delivery vehicle can be cancerous or non-cancerous. A disease can be familial adenomatous polyposis (FAP), attenuated FAP, cancer, chronic inflammatory bowel disease, chronic inflammatory bowel disease, ileal Crohn's or any combination thereof. In some cases, a disease can be identified by genetic screening. For example, a genetic screen can identify a BCRA mutation in a subject that can predispose them to breast cancer. In other cases, a genetic screen can identify a mutation in an APC gene that can result in FAP. A disease can also be for example, an ocular disease, a reproductive disease, a gastrointestinal disease, A disease can be a genetic disease. A disease can produce polyps in a gastrointestinal tract. In some cases, a disease is FAP. FAP can progress to cancer. A gastrointestinal disease can be hereditary. For example, a hereditary gastrointestinal disease can be Gilbert's syndrome, telangiectasia, mucopolysaccaride, Osler-Weber-Rendu syndrome, pancreatitis, keratoacanthoma, biliary atresia, Morquio's syndrome, Hurler's syndrome, Hunter's syndrome, Crigler-Najjar, Rotor's, Peutz-Jeghers' syndrome, Dubin-Johnson, Osteochondroses, Osteochondrodysplasias, polyposis, or a combination thereof.

For oral administration, an excipient may include pharmaceutical grades of mannitol, lactose, starch, magnesium stearate, sodium saccharine, talcum, cellulose, glucose, gelatin, sucrose, magnesium carbonate, and the like. If desired, a liposomal composition may also contain minor amounts of non-toxic auxiliary substances such as wetting agents, emulsifying agents, or buffers.

A composition can be administered orally, by subcutaneous or other injection, intravenously, intracerebrally, intramuscularly, parenterally, transdermally, nasally or rectally. The form in which the compound or composition is administered depends at least in part on the route by which the compound is administered. In some cases, a liposomal composition can be employed in the form of solid preparations for oral administration; preparations may be tablets, granules, powders, capsules or the like. In a tablet formulation, a composition is typically formulated with additives, e.g. an excipient such as a saccharide or cellulose preparation, a binder such as starch paste or methyl cellulose, a filler, a disintegrator, and other additives typically used in the manufacture of medical preparations. Methods for preparing such dosage forms may be apparent to those skilled in the art. A liposomal composition to be administered may contain a quantity of a nanoparticle in a pharmaceutically effective amount for therapeutic use in a biological system, including a patient or subject. A pharmaceutical composition may be administered daily or administered on an as needed basis. In certain embodiments, a pharmaceutical composition can be administered to a subject prior to bedtime. In some embodiments, a pharmaceutical composition can be administered immediately before bedtime. In some embodiments, a pharmaceutical composition can be administered within about two hours before bedtime, preferably within about one hour before bedtime. In another embodiment, a pharmaceutical composition can be administered about two hours before bedtime. In a further embodiment, a pharmaceutical composition can be administered at least two hours before bedtime. In another embodiment, a pharmaceutical composition can be administered about one hour before bedtime. In a further embodiment, a pharmaceutical composition can be administered at least one hour before bedtime. In a still further embodiment, a pharmaceutical composition can be administered less than one hour before bedtime. In still another embodiment, the pharmaceutical composition can be administered immediately before bedtime. A pharmaceutical composition is administered orally or rectally.

An appropriate dosage (“therapeutically effective amount”) of an active agent(s) in a composition may depend, for example, on the severity and course of a condition, a mode of administration, a bioavailability of a particular agent(s), the age and weight of a subject, a subject's clinical history and response to an active agent(s), discretion of a physician, or any combination thereof. A therapeutically effective amount of an active agent(s) in a composition to be administered to a subject can be in the range of about 100 μg/kg body weight/day to about 1000 mg/kg body weight/day whether by one or more administrations. In some embodiments, the range of each active agent administered daily can be from about 100 μg/kg body weight/day to about 50 mg/kg body weight/day, 100 μg/kg body weight/day to about 10 mg/kg body weight/day, 100 μg/kg body weight/day to about 1 mg/kg body weight/day, 100 μg/kg body weight/day to about 10 mg/kg body weight/day, 500 μg/kg body weight/day to about 100 mg/kg body weight/day, 500 μg/kg body weight/day to about 50 mg/kg body weight/day, 500 μg/kg body weight/day to about 5 mg/kg body weight/day, 1 mg/kg body weight/day to about 100 mg/kg body weight/day, 1 mg/kg body weight/day to about 50 mg/kg body weight/day, 1 mg/kg body weight/day to about 10 mg/kg body weight/day, 5 mg/kg body weight/dose to about 100 mg/kg body weight/day, 5 mg/kg body weight/dose to about 50 mg/kg body weight/day, 10 mg/kg body weight/day to about 100 mg/kg body weight/day, and 10 mg/kg body weight/day to about 50 mg/kg body weight/day.

As used herein, “pharmaceutically acceptable carrier” includes any and all solvents, dispersion media, coatings, antibacterial and antifungal agents, isotonic and absorption delaying agents, sweeteners, salts, buffers, and the like. The pharmaceutically acceptable carriers may be prepared from a wide range of materials including, but not limited to, flavoring agents, sweetening agents and miscellaneous materials such as buffers and absorbents that may be needed in order to prepare a particular therapeutic composition.

The compositions described herein can be formulated under sterile conditions within a reasonable time prior to administration. In some cases, a secondary therapy can also be administered. For example, another therapy such as chemotherapy or radiation therapy may be administered before or subsequent to the administration of the complex, for example within 12 hr. to 7 days. A combination of therapies, such as both chemotherapy and radiation therapy may be employed in addition to the administration of the complex. Other therapies for use with the compositions and methods herein include the use of chemotherapeutic agents, cytotoxic/antineoplastic agents, anti-angiogenic agents and other known cancer therapeutics, small molecules and biologics.

### Method of Use

The compositions herein can be used for therapeutic and diagnostic applications. In some embodiments, the compositions described herein are employed as a diagnostic to monitor a therapy for a disease or condition affecting a cell or tissue that has a mucus-layer. The compositions and methods herein provide a means for delivering a diagnostic agent through the mucus layer to reach the target cells or tissue. As an example of such diagnostic, the compositions herein can be used as a diagnostic for familial adenomatous polyposis (FAP) or other disease state in a patient. A patient may be administered an effective amount of composition that includes a mucus-penetrating delivery vehicle as well as an MPP, and a diagnostic method may include determining a level of cargo incorporated into a cell genome whereupon a difference in cargo levels before the start of therapy in a patient and during and/or after therapy will evidence the effectiveness of therapy in a patient, including whether a patient has completed therapy or whether the disease state has been inhibited or eliminated.

In other cases, the compositions described herein may be administered to a subject as a preventive measure. For example, a subject may not have diagnosed disease and may appear to be predisposed to a disease such as cancer, such as colon cancer, where the affected cell or tissue has a mucus-layer. The compositions and methods herein provide a means for delivering the preventative agent through the mucus layer to reach the target cells or tissue. In some cases, the compositions described herein may be administered to a subject to treat an existing disease or condition, particularly where the cell or tissue targeted for the therapeutic delivery has a mucus-layer

In some cases the composition employed contains a cargo that is delivered to the cell and can then genetically modify the target cell(s). For example, a polynucleic acid may transduce a cell that it contacts. An efficiency of transduction or transfection with a polynucleic acid described herein, for example, can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted. An efficiency of cellular uptake with a structure, such as the compositions described herein having a mucus-penetrating delivery vehicle with an MPP can permit efficient penetration and transit through the mucus layer to the target cells and thereby have an efficient uptake by the target cell(s), for example, uptake can be or can be about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.5%, 99.9%, or more than 99.9% of the total number of cells that are contacted. In some cases, the compositions can have a higher percent of cellular uptake as compared to a comparable delivery vehicle that does include an MPP. The improvement over a non-MPP containing composition can be from about 10%, 15%, 20%, 25%, 30%, 35%, 40%, 45%, 50%, 55%, 60%, 65%, 70%, or up to about 80% better. In some cases, an efficiency of transfection or integration of a polynucleic acid cargo delivered to a cell by an MPP-containing delivery vehicle composition can be from about 5%, 10%, 15%, 20%, 25%, 30%, 35%, 40%, 45%, 50%, 55%, 60%, or up to 65% better than a comparable delivery vehicle that does not include an MPP.

The compositions provided herein for delivering a cargo can be functional for at least or at least about 1, 2, 3, 4, 5, 6, 7, 8, 9, 10, 11, 12, 13, 14, 15, 16, 17, 18, 19, 20, 21, 22, 23, 24, 25, 6, 27, 28, 29, 30, 40, 50, 60, 70, 80, 90, or 100 days after introduction to a subject in need thereof. Structures can be functional for at least or at least about 1, 2, 3, 4, 5, 6, 7, 8, 9, 10, 11, or 12 months after introduction into a subject. A structure, such as a liposome, can be functional for at least or at least about 1, 2, 3, 4, 5, 6, 7, 8, 9, 10, 15, 20, 25, or 30 years after introduction to a subject. In some cases, a liposome can be functional for up to the lifetime of a recipient. Further, a structure such as a liposome can function at 100% of its normal intended operation. Liposomes can also function 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% of their normal intended operation. Function of a liposome may refer to the efficiency of delivery, persistence of a liposome, stability of a liposome, or any combination thereof.

The compositions provided herein can deliver a cargo, such as a minicircle DNA vector, to a target cell. In some cases, function can include a percent of cells that received a minicircle DNA vector from the delivery vehicle composition. In other cases, function can refer to a frequency or efficiency of protein generation from a polynucleic acid. For example, a delivery vehicle composition may deliver a vector to a cell that encodes for at least a portion of a gene, such as APC. A frequency of efficiency of APC generation from a vector may describe a functionality of a vector or liposome.

A minicircle vector concentration can be from 0.5 nanograms to 50 micrograms. A minicircle vector concentration can be from about 0.5 ng, 1 ng, 2 ng, 5 ng, 10 ng, 50 ng, 100 ng, 150 ng, 200 ng, 300 ng, 400 ng, 500 ng, 600 ng, 700 ng, 800 ng, 900 ng, 1000 ng, 1 μg, 2 μg, 5 μg, 10 μg, 20 μg, 30 μg, 40 μg, 50 μg, 60 μg, or up to 50 μg or greater. In some cases, the amount of nucleic acid (e.g., ssDNA, dsDNA, RNA) that may be introduced to a cell by a structure may be varied to optimize transfection efficiency and/or cell viability. In some cases, less than about 100 picograms of nucleic acid may be introduced to a subject. In some cases, at least about 100 picograms, at least about 200 picograms, at least about 300 picograms, at least about 400 picograms, at least about 500 picograms, at least about 600 picograms, at least about 700 picograms, at least about 800 picograms, at least about 900 picograms, at least about 1 microgram, at least about 1.5 micrograms, at least about 2 micrograms, at least about 2.5 micrograms, at least about 3 micrograms, at least about 3.5 micrograms, at least about 4 micrograms, at least about 4.5 micrograms, at least about 5 micrograms, at least about 5.5 micrograms, at least about 6 micrograms, at least about 6.5 micrograms, at least about 7 micrograms, at least about 7.5 micrograms, at least about 8 micrograms, at least about 8.5 micrograms, at least about 9 micrograms, at least about 9.5 micrograms, at least about 10 micrograms, at least about 11 micrograms, at least about 12 micrograms, at least about 13 micrograms, at least about 14 micrograms, at least about 15 micrograms, at least about 20 micrograms, at least about 25 micrograms, at least about 30 micrograms, at least about 35 micrograms, at least about 40 micrograms, at least about 45 micrograms, or at least about 50 micrograms, of nucleic acid may be added to each cell sample (e.g., one or more cells being electroporated). In some cases, the amount of nucleic acid (e.g., dsDNA) required for optimal transfection efficiency and/or cell viability may be specific to the cell type.

In some cases, an effective amount of a structure can mean an amount sufficient to increase the expression level of at least one gene which can be decreased in a subject prior to the treatment or an amount sufficient to alleviate one or more symptoms of cancer. For example, an effective amount can be an amount sufficient to increase the expression level of at least one gene selected from the group consisting of gastrointestinal differentiation genes, cell cycle inhibition genes, and tumor suppressor genes by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

In some embodiments, an effective amount means an amount sufficient to decrease the expression level of at least one gene which may be increased in the subject prior to the treatment or an amount sufficient to alleviate one or more symptoms of cancer. For example, an effective amount can be an amount sufficient to decrease the expression level of a gene by at least 5%, 10%, 15%, 20%, 25%, 30%, 35%, 40%, 45%, 50%, 55%, 60%, 65%, 70%, 75%, 80%, 85%, 90%, 95%, 100%, 200%, 300%, 400%, 500%, 1000%, 1500%, or more compared to a reference value or the expression level without the treatment of any compound.

An effective amount for a subject will depend upon the subject's body weight, size, and health; the nature and extent of the condition; and the therapeutic selected for administration. An effective amount for a given situation can be determined by routine experimentation that may be within the skill and judgment of a clinician. An effective amount, as used herein, can refer to an amount of delivery vehicle composition sufficient to produce a measurable biological response (e.g., presence of cargo and/or cargo biological activity in a cell). Actual dosage levels of the delivery vehicle composition can be varied so as to administer an amount that may be effective to achieve the desired response for a particular subject and/or application. The selected dosage level will depend upon a variety of factors including the type of tissue being addressed, the types of cells, combination with other drugs or treatments, severity of the condition being treated, and the physical condition and prior medical history of the subject being treated. Preferably, a minimal dose can be administered, and a dose can be escalated in the absence of dose-limiting toxicity to a minimally effective amount.

A polynucleic acid cargo delivered by a delivery vehicle composition may encode for a tumor-suppressor gene. A tumor-suppressor gene can generally encode for a protein that in one way or another can inhibit cell proliferation. Loss of one or more of these “brakes” may contribute to the development of a cancer. In some cases, introducing a tumor suppressor gene encoding for a protein may ameliorate disease, prevent disease, or treat disease in a subject.

In some cases, a subject who inherits a mutant allele of APC, a tumor-suppressor gene, may have a high risk of developing colon cancer. Inheriting one mutant allele of another tumor-suppressor gene increase to almost 100 percent the probability that a subject will develop a specific tumor. In some cases, a subject that has inherited a mutant allele of APC, or a tumor-suppressor gene, may receive delivery vehicle composition described herein. In some cases, the delivery vehicle composition may contain a cargo polynucleic acid encoding for a protein produced by a mutant allele inherited in a subject. A mutant allele can be a tumor-suppressor protein such as APC. A protein can also be GLB1, DEFA5, WAC, DEFA6, or a combination thereof. Additional tumor-suppressor genes can be delivered. In some cases, a tumor suppressor can be a WW domain-containing adaptor with coiled-coil (WAC) gene.

Suitable formulations can include aqueous and non-aqueous sterile injection solutions that can contain antioxidants, buffers, bacteriostats, bactericidal antibiotics and solutes that render the formulation isotonic with the bodily fluids of the intended recipient; and aqueous and non-aqueous sterile suspensions, which can include suspending agents and thickening agents. Suitable inert carriers can include sugars such as lactose. In some cases, the compositions can take such forms as suspensions, solutions or emulsions in oily or aqueous vehicles, and can contain formulatory agents such as suspending, stabilizing and/or dispersing agents. Alternatively, the active ingredient can be in powder form for constitution with a suitable vehicle, e.g., sterile pyrogen-free water, before use.

A carrier can be a solvent or dispersion medium containing, for example, water, ethanol, one or more polyols (e.g., glycerol, propylene glycol, and liquid polyethylene glycol), oils, such as vegetable oils (e.g, peanut oil, corn oil, sesame oil, etc.), and combinations thereof. The proper fluidity can be maintained, for example, by the use of a coating, such as lecithin, by the maintenance of the required particle size in the case of dispersion and/or by the use of surfactants. In many cases, it will be preferable to include isotonic agents, for example, sugars or sodium chloride. Solutions and dispersions of the active compounds as the free acid or base or pharmacologically acceptable salts thereof can be prepared in water or another solvent or dispersing medium suitably mixed with one or more pharmaceutically acceptable excipients including, but not limited to, surfactants, dispersants, emulsifiers, pH modifying agents, and combination thereof. Suitable surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimoniuni bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-beta-alanine, sodium N-lauryl-beta-iminodipropionate, myristoamphoacetate, lauryl betaine and lauryl sulfobetaine. The formulation can contain a preservative to prevent the growth of microorganisms. Suitable preservatives include, but are not limited to, parabens, chlorobutanol, phenol, sorbic acid, and thimerosal. The formulation may also contain an antioxidant to prevent degradation of the active agent(s). The formulation is typically buffered to a pH of 3-8 for parenteral administration upon reconstitution. Suitable buffers include, but are not limited to, phosphate buffers, acetate buffers, and citrate buffers. Water soluble polymers can be often used in formulations for parenteral administration. Suitable water-soluble polymers include, but are not limited to, polyvinylpyrrolidone, dextran, carboxymethylcellulose, and polyethylene glycol.

Sterile injectable solutions can be prepared by incorporating the active compounds in the required amount in the appropriate solvent or dispersion medium with one or more of the excipients listed above, as required, followed by filtered sterilization. Generally, dispersions can be prepared by incorporating the various sterilized active ingredients into a sterile vehicle which contains the basic dispersion medium and the required other ingredients from those listed above. In the case of sterile powders for the preparation of sterile injectable solutions, a method of preparation can be vacuum-drying and freeze-drying techniques which yield a powder of the active ingredient plus any additional desired ingredient from a previously sterile-filtered solution thereof. The powders can be prepared in such a manner that the particles are porous in nature, which can increase dissolution of the particles. Methods for making porous particles are well known in the art.

A formulation can be an ocular formulation or a topical formation. Pharmaceutical formulations for ocular administration can be in the form of a sterile aqueous solution or suspension of particles formed from one or more polymer-drug conjugates. Acceptable solvents include, for example, water, Ringer's solution, phosphate buffered saline (PBS), and isotonic sodium chloride solution. The formulation may also be a sterile solution, suspension, or emulsion in a nontoxic, parenterally acceptable diluent or solvent such as 1,3-butanediol. In still other embodiments, the delivery vehicle composition can be formulated for topical administration to mucosa. Suitable dosage forms for topical administration include creams, ointments, salves, sprays, gels, lotions, emulsions, liquids, and transdermal patches. The formulation may be formulated for transmucosal, transepithelial, transendothelial, or transdermal administration. The compositions contain one or more chemical penetration enhancers, membrane permeability agents, membrane transport agents, emollients, surfactants, stabilizers, and combination thereof. In some embodiments, the delivery vehicle composition can be administered as a liquid formulation, such as a solution or suspension, a semi-solid formulation, such as a lotion or ointment, or a solid formulation. In some embodiments, the delivery vehicle composition can be formulated as liquids, including solutions and suspensions, such as eye drops or as a semi-solid formulation, such as ointment or lotion for topical application to mucosa, such as the eye or vaginally or rectally. The formulation may contain one or more excipients, such as emollients, surfactants, emulsifiers, and penetration enhancers.

In some cases, formulations can be presented in unit-dose or multi-dose containers, for example sealed ampoules and vials, and can be stored in a frozen or freeze-dried (lyophilized) condition requiring only the addition of sterile liquid carrier immediately prior to use. For oral administration, the compositions can take the form of, for example, tablets or capsules prepared by a conventional technique with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). The tablets can be coated in some cases. Liquid preparations for oral administration can take the form of, for example, solutions, syrups or suspensions, or they can be presented as a dry product for constitution with water or other suitable vehicle before use. Such liquid preparations can be prepared by conventional techniques with pharmaceutically acceptable additives such as suspending agents (e.g., sorbitol syrup, cellulose derivatives or hydrogenated edible fats); emulsifying agents (e.g. lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations can also contain buffer salts, flavoring, coloring and sweetening agents as appropriate. Preparations for oral administration can be suitably formulated to give controlled release of the active compound. For buccal administration the compositions can take the form of tablets or lozenges formulated in conventional manner. In some cases, compositions can also be formulated as a preparation for implantation or injection. Thus, for example, a structure can be formulated with suitable polymeric, aqueous, and/or hydrophilic materials, or resins, or as sparingly soluble derivatives (e.g., as a sparingly soluble salt). The compounds can also be formulated in rectal compositions, creams or lotions, or transdermal patches.

In some cases, a pharmaceutical composition may include a salt. A salt can be relatively non-toxic. Examples of pharmaceutically acceptable salts include those derived from mineral acids, such as hydrochloric acid and sulfuric acid, and those derived from organic acids, such as ethanesulfonic acid, benzenesulfonic acid, p-toluenesulfonic acid, and the like. Examples of suitable inorganic bases for the formation of salts include the hydroxides, carbonates, and bicarbonates of ammonia, sodium, lithium, potassium, calcium, magnesium, aluminum, zinc and the like. Salts may also be formed with suitable organic bases, including those that are non-toxic and strong enough to form such salts. For purposes of illustration, the class of such organic bases may include mono-, di-, and trialkylamines, such as methylamine, dimethylamine, and triethylamine; mono-, di- or trihydroxyalkylamines such as mono-, di-, and triethanolamine; amino acids, such as arginine and lysine; guanidine; N-methylglucosamine; N-methylglucamine; L-glutamine; N-methylpiperazine; morpholine; ethylenediamine; N-benzylphenethylamine; (trihydroxymethyl)aminoethane; and the like.

In some cases, delivery vehicle compositions can have a circulation half-life in a subject of about 6 hours, 12 hours, 18 hours, 24 hours, 30 hours, 36 hours, 42 hours, or 48 hours. In some embodiments the nanoparticles can comprise a circulation half-life of more than about 48 hours. In some embodiments circulation half-life can be enhanced by increasing the concentration of a hydrophobic monomer of the polymer, thereby increasing the forces necessary to disassemble the nanostructures.

In some cases, a level of disease can be determined in sequence or concurrent with a delivery vehicle composition regime. A level of disease on target lesions can be measured as a Complete Response (CR): Disappearance of all target lesions, Partial Response (PR): At least a 30% decrease in the sum of the longest diameter (LD) of target lesions taking as reference the baseline sum LD, Progression (PD): At least a 20% increase in the sum of LD of target lesions taking as reference the smallest sum LD recorded since the treatment started or the appearance of one or more new lesions, Stable Disease (SD): Neither sufficient shrinkage to qualify for PR nor sufficient increase to qualify for PD taking as references the smallest sum LD. In other cases, a non-target lesion can be measured. A level of disease of a non-target lesion can be Complete Response (CR): Disappearance of all non-target lesions and normalization of tumor marker level, Non-Complete Response: Persistence of one or more non-target lesions, Progression (PD): Appearance of one or more new lesions. Unequivocal progression of existing non-target lesions.

### Kits

Disclosed herein can be kits comprising delivery vehicle compositions. In some cases, a kit can include a therapeutic or prophylactic delivery vehicle composition containing an effective amount of a cargo in unit dosage form. In some cases, a kit comprises a sterile container which can contain a delivery vehicle composition including a cargo; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments. In some cases, a delivery vehicle composition can be dehydrated, stored and then reconstituted such that a substantial portion of an internal content is retained.

### EXAMPLES

### Example 1: Cell-Penetration Assay

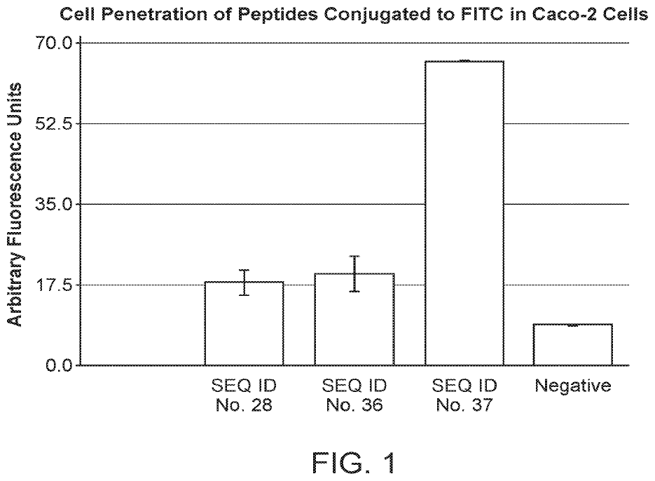

The peptides SEQ ID NO:28 (TVDNPASTTNKDKLFAV), SEQ ID NO: 36 (LIIYRDLISH) and Tat SEQ ID NO: 37 (GRKKRRQRRRPQ) were synthesized with a PEG2-FITC modification on their N-terminus for fluorescent imaging. Caco-2 cells were plated in a 24-well plate, incubated with 10 uM of each peptide in triplicates and incubated for 1 h at 37 C. Cells were washed three times with PBS and imaged using a Keyence BZ-X700 fluorescent microscope. Images were used to quantify fluorescence from each well. Data is shown in FIG. 1.

All three peptides were found to have a higher penetration than the negative control.

### Example 2: Peptide Screen: Fauchere Study and Hodges Study

Fauchere Study:

Candidate peptides were screened by Fauchere study for average hydropathy per residue scores below 0.5 (Fauchere study) at pH 7 in the absence of salt. Candidate peptides were used in the analysis. A Fauchere score was calculated by adding the Fauchere per residue score, as described in Table 1, of each amino acid residue of a peptide.

Fauchere Score=Sum of Fauchere per residue score. The sequence, MATKGGTVKA, for example corresponds to the sum of: 1.230+0.310+0.260+−0.990+0+0+0.260+1.220+−0.990+0.310=1.61.

The Fauchere score per residue corresponds to the Fauchere Score divided by the total number of amino acid residues. The Fauchere score per residue for MATKGGTVKA is: 1.61/10=0.161.

Hodges Study:

Candidate peptides were screened by Hodges study for average hydropathy per residue scores below 10 at pH 7 in the absence of salt. Candidate peptides were used in the analysis. A Hodges score was calculated by adding the Hodges per residue score, as described in Table 2, of each amino acid residue of a peptide.

Hodges score=Sum of Hodges per residue score. The sequence, MATKGGTVKA, for example corresponds to the sum of: 16.3+3.9+3.9+−1.1+0+0+3.9+14.4+−1.1+3.9=44.1.

The Hodges score per residue corresponds to the Hodges Score divided by the total number of amino acid residues. The Hodges score per residue for MATKGGTVKA is: 44.4/10=4.41.

### Example 3: Delivery Vehicle Preparation: Liposomal Vehicle with Surface Modification

DSPE-PEG2000 (N-(Methylpolyoxyethylene oxycarbonyl)-1,2-distearoyl-sn-glycero-3-phosphoethanolamine) with the terminal end group of PEG2000 modified with a maleimide can be used to conjugate a peptide with an added thiol in the form of a cysteine amino acid. Alternatively, another covalent conjugation method can be used, such as click or amide chemistry.

To generate the mucus penetrating delivery vehicle, MVL5(N1-[2-((1S)-1-[(3-aminopropyl)amino]-4-[di(3-amino-propyl)amino]butylcarboxamido)ethyl]-3,4-di[oleyloxy]-benzamide)/DOPE (dioleoylphosphatidylethanolamine)/DSPE-PEG2000/DSPE-PEG2000-peptide can be combined in chloroform at a 50/43/9/1% ratio. A separate control vehicle (MPP-Control) can be made with no DSPE-PEG2000-peptide and 10% mol DSPE-PEG2000. The DSPE-PEG2000 is present in a “brush” configuration at 10% mol ratio at which it PEG provides it with a mucus penetrating property. The peptide hangs on the PEG exposed to the surface. After mixing the lipid solutions in methanol: chloroform solution, the mixture can be dried in vacuum into a thin-film. The appropriate amount of sterile, high resistivity (18.2 MΩcm) water can be used to achieve a final concentration of 1 mM of lipid. The resulting mixture can be incubated at 37 degrees Celsius for 12 hours to form liposomes. Following the incubation, the liposome solution can be extruded with 20 passes through a 200 nm polycarbonate pore.

Using dynamic light scattering, nanoparticle size can be determined and the ideal near neutral zeta potential, which indicates that the surface can be sufficiently PEGylated, can be measured by laser Doppler anemometry.

### Example 4: Cargo Loading

EGFP DNA can be loaded into the cargo by diluting the DNA and delivery vehicle in a suitable solvent, such as OPTI-MEM or a mixture of water and ethanol, and adding the DNA to the delivery vehicle dropwise and letting the solution rest for 20 minutes. A charge ratio of +5 can be used for the carrier to DNA ratio.

### Example 5: Dynamic Light Scattering and Zeta Potential

The size and effective charge measurement of DNA vehicle nanoparticles can be measured using a Malvern Nanosizer ZS (Malvern Instruments). The nanoparticles can be prepared in light-scattering vials at a charge ratio of +5 suspended in 1 mL of the appropriate buffer and incubated at room temperature for 20 minutes.

### Example 6: In Vitro Transfection of Caco-2 Cells

Human Colorectal adenocarcinoma, Caco-2 cells (ATCC number: HTB-37) can be cultured in ATCC-formulated Eagle's Minimum Essential Medium supplemented with 10% fetal bovine serum (HyClone) and 1% Penicillin/Streptomyocin (Invitrogen). Cells can be kept at 37° C. in a humidified atmosphere containing 5% CO2 and can be reseeded every 72 h to maintain subconfluency. For transfection studies, cells can be seeded in 24 well-plates such that confluency at transfection can be 60-80%. EGFP-DNA nanostructures can be formed by diluting 1 μg of DNA and the appropriate amount of liposome solution to 250 μL each with Optimem (Invitrogen) and mixing. Nanostructures can be incubated for 20 minutes at room temperature before addition to cells. Cells can be subsequently washed once with PBS and then incubated with 200 μL of complex suspension (0.4 μg of DNA per well) for 6 h. After 6 h, the transfection medium can be removed, and the cells can be rinsed once with PBS and then incubated in supplemented DMEM for 18 h. Transfection efficiency can be measured using a fluorescent microscope and images analyzed to assess fluorescent intensity and number of cells positive for GFP. If the transfection efficiency of the peptide conjugated nanoparticle is higher than the fluorescent intensity of the nanoparticle then the peptide is considered to have cell penetration property.

### Example 7: Mucus Penetration

Fresh porcine intestines can be attained at an abattoir. Square sections of 2 cm×2 cm can be carved from the intestines and the nanoparticles carrying 4 micrograms of fluorescent-labelled DNA (such as Cy5-labelled 60-mer DNA from Integrated DNA Technologies) can be dropped on them. After 60 min of incubation, the intestinal slices can be embedded in OCT, cryofrozen and sectioned. Fluorescent microscopy can be used to quantify and determine the distance travelled by the nanoparticles in the mucus. If the peptide conjugated nanoparticle has same or more penetration in the mucus than the control vehicle than the peptide is considered mucus penetrating.

### Example 8: Large Screen Cell Penetration Assay

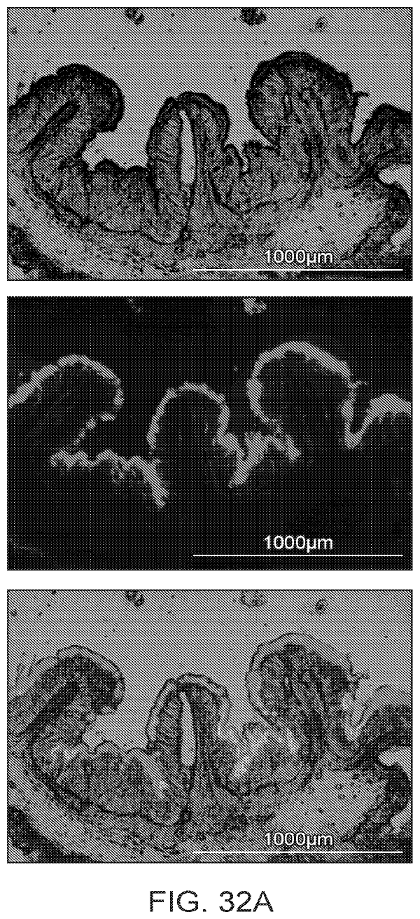

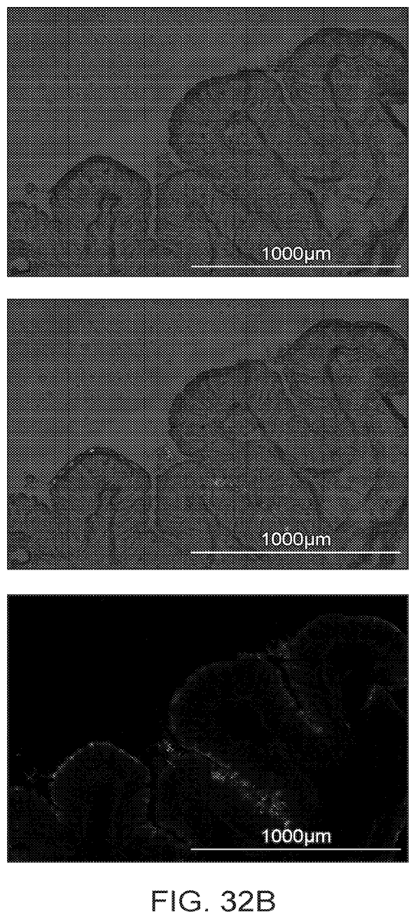

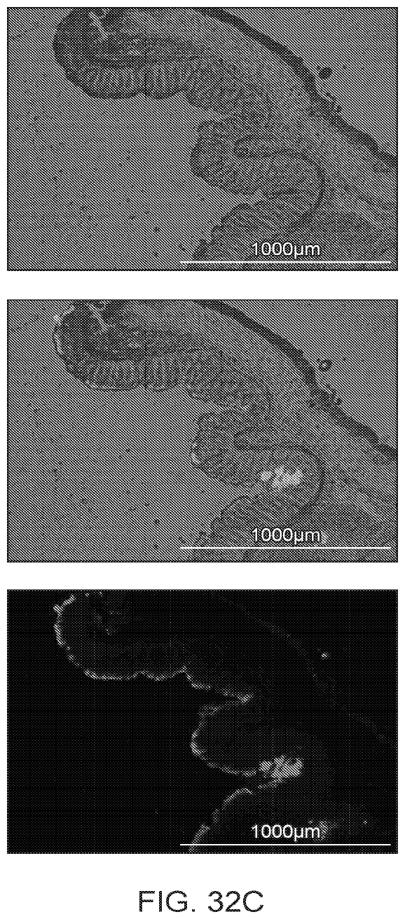

Peptides were synthesized and conjugated with a PEG2-FITC modification on their N-terminus for fluorescent imaging. Caco-2 cells were plated in a 24-well plate, incubated with 10 μM concentration of each peptide in triplicates and incubated for 1 h at 37° C. Cells were washed three times using 0.5 mg/mL of heparin sulfate in PBS wash. Cells were imaged using a BioTek Cytation 3 imager and the number and intensity of FITC positive cells was analyzed using the imager's software. Results are shown in FIG. 32.

### Example 9: Peptide Mucin Interaction Assay

DSPE-PEG2k-DBCO (Avanti Polar Lipids) was hydrated in water and agitated to form a transparent solution. Synthesized peptides with a lysine containing an azide on the N-terminus were conjugated to it. 2.5× moles of the azide peptides were added to the lipid mix and were left to react overnight. 10× moles of sodium azide were added to the reaction to quench it.

Thin film hydration was used to make a lipid base system composed of MVL5/DOPC/Chol (30/60/10% mol). Briefly, the lipids were dissolved in chloroform: methanol (9:1) and mixed. The lipids were then dried using a rotovap and hydrated in a HEPES-glucose buffer (10 mM HEPES, 230 mM Glucose, pH 7.4) for a final concentration of 1 mM. The lipid suspension was extruded through a 200 nm pore size filter for 20 passes using the NanoSizer MINI (T&T Scientific Corporation, Tennessee). An appropriate amount of DNA was added to the lipid base system for a charge ratio of +3 (assuming MVL5 has a charge ratio of +3 at neutral pH) and the solution was mixed thoroughly and let rest for 20 min. DSPE-PEG2k-DBCO conjugated peptides were added to the base system for a final lipid concentration of 0.08% mol. DSPE-SS-PEG2k was added to the base system for a final lipid concentration of 5% mol. The solutions were incubated for 1 h at 60° C.

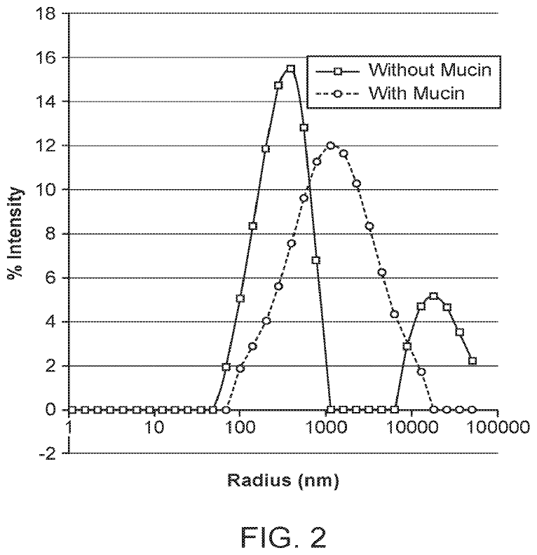

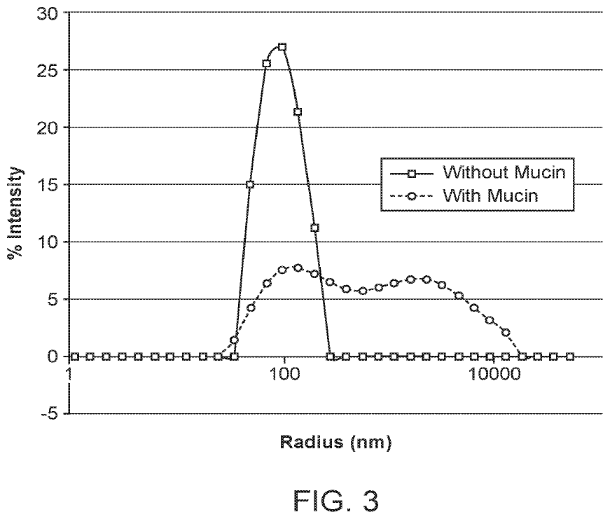

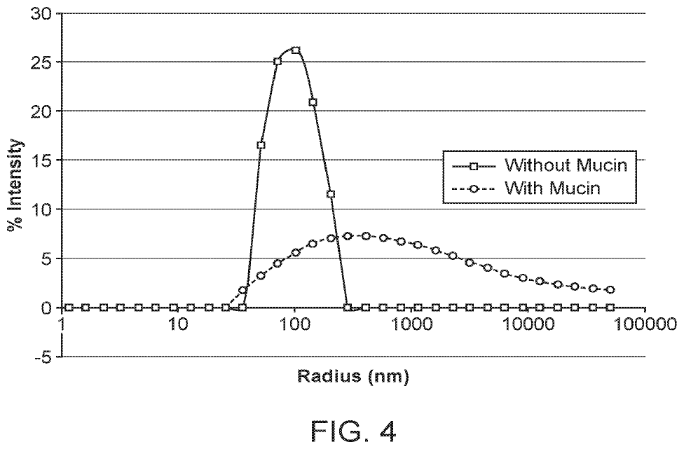

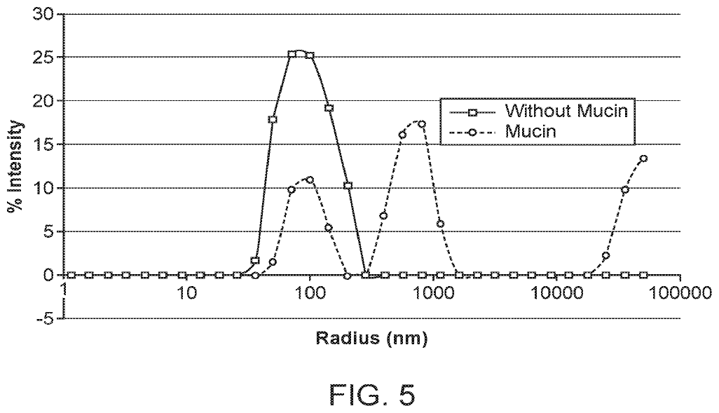

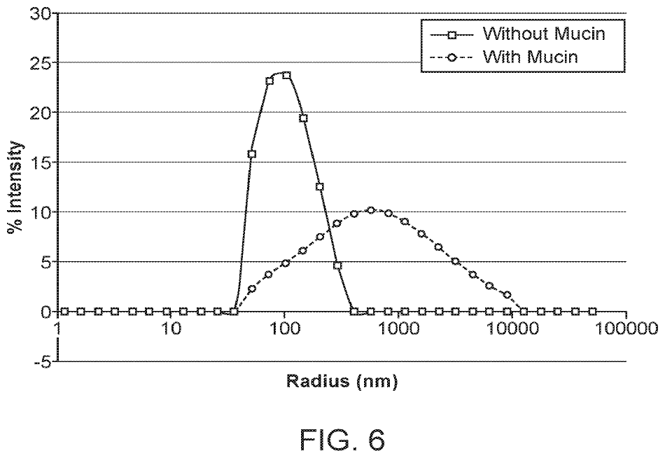

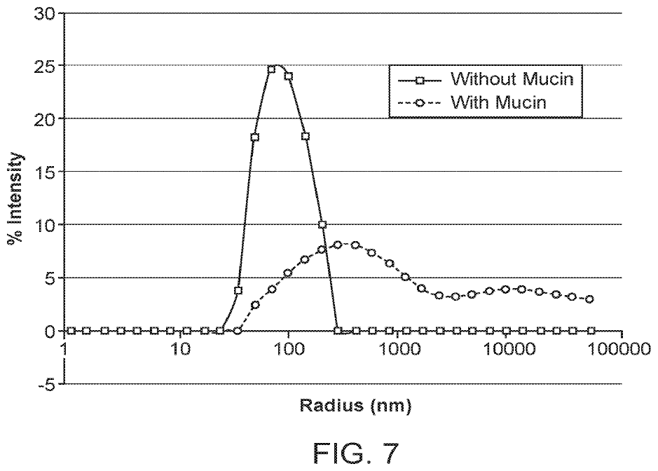

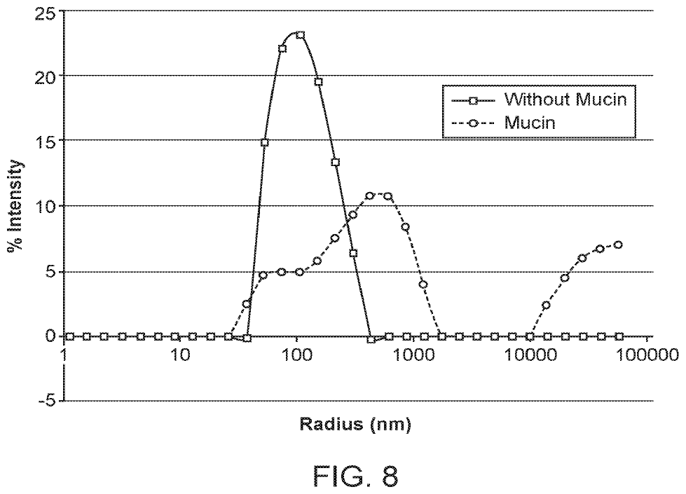

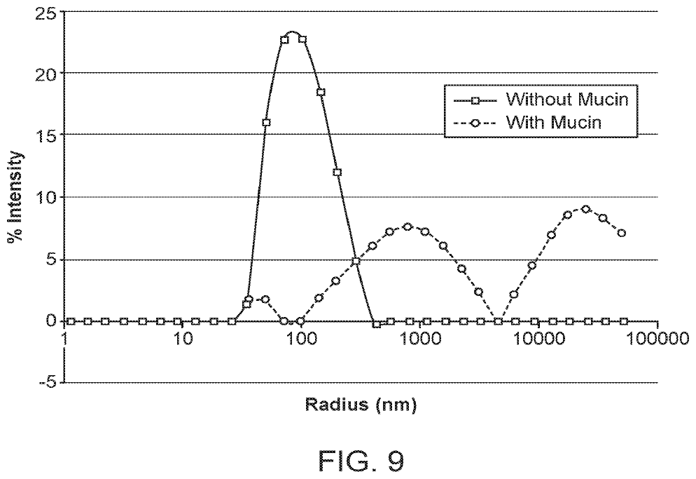

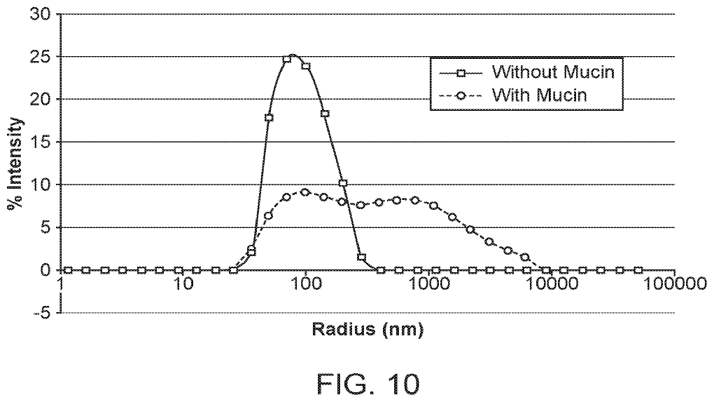

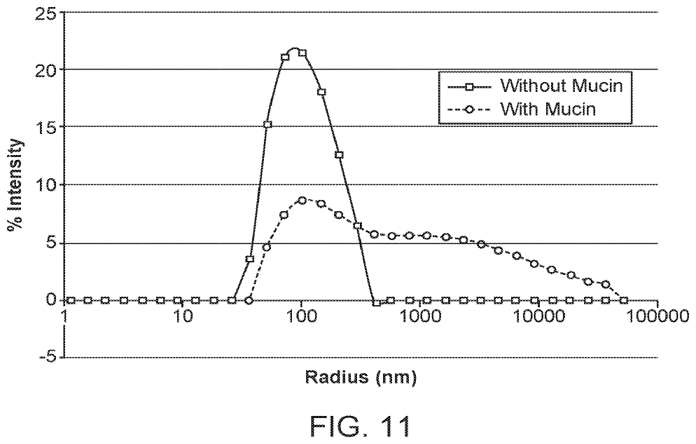

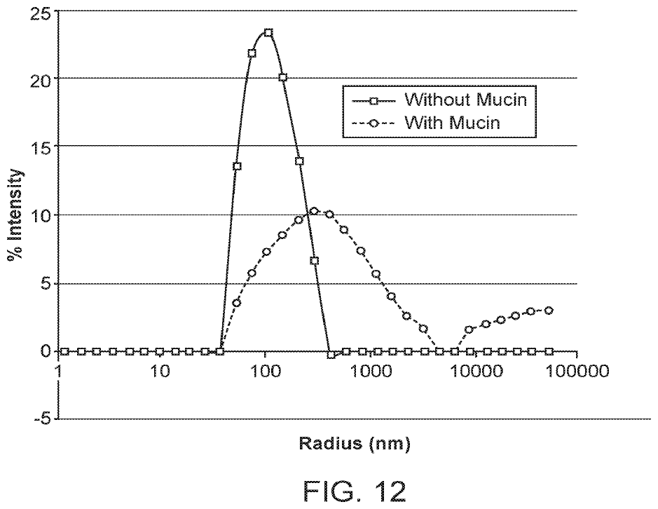

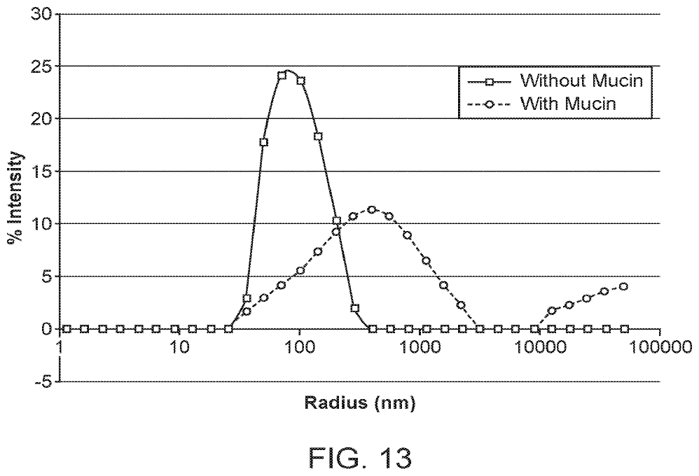

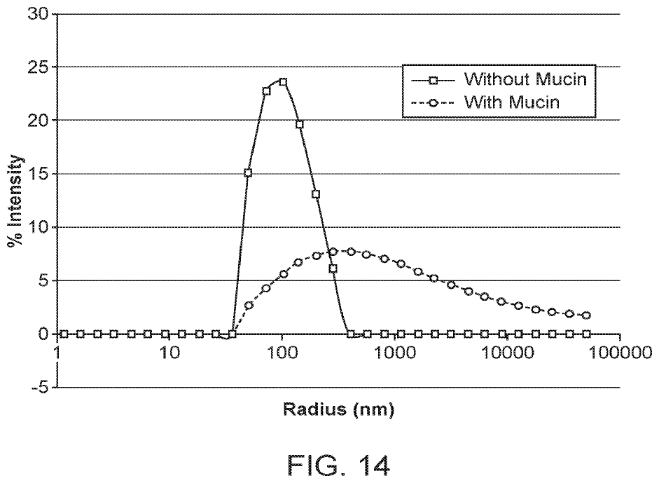

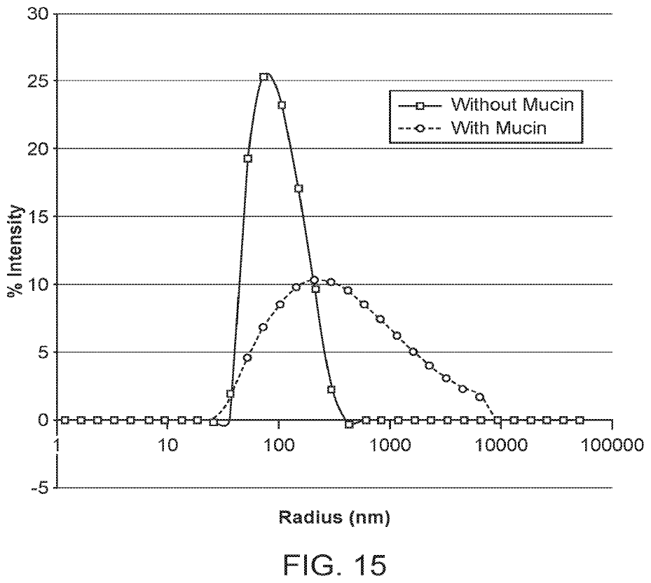

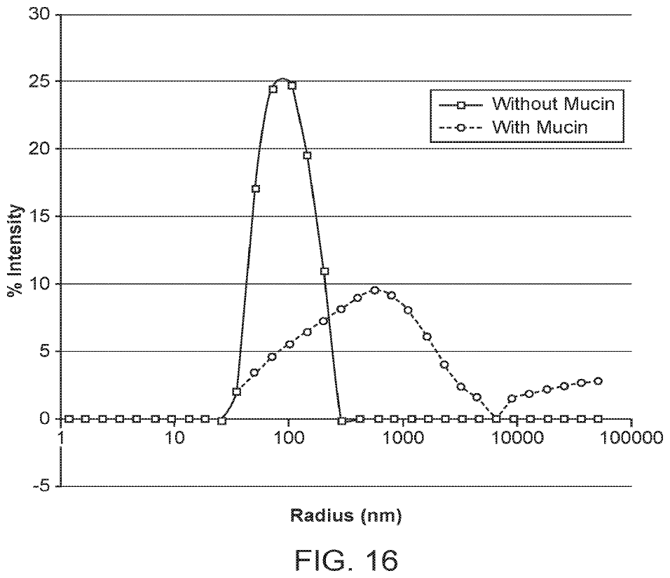

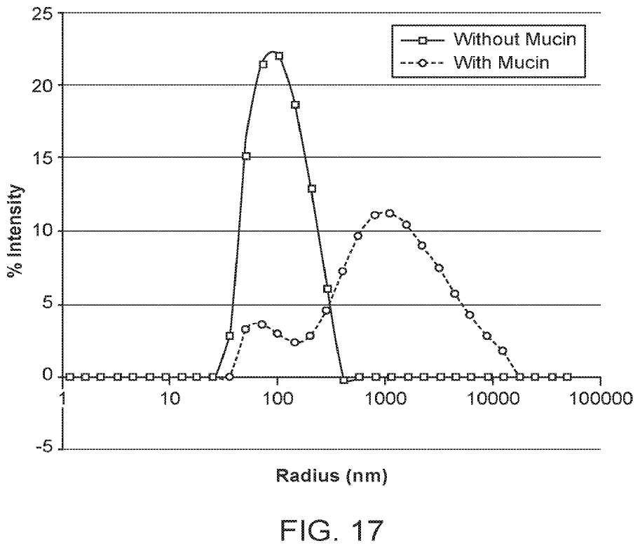

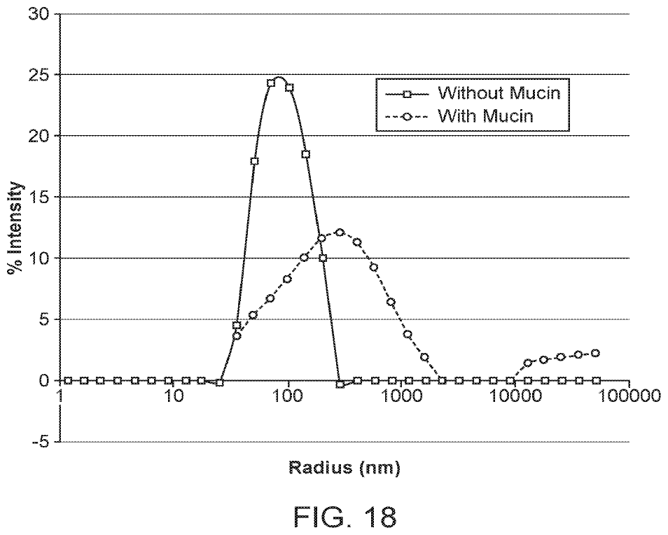

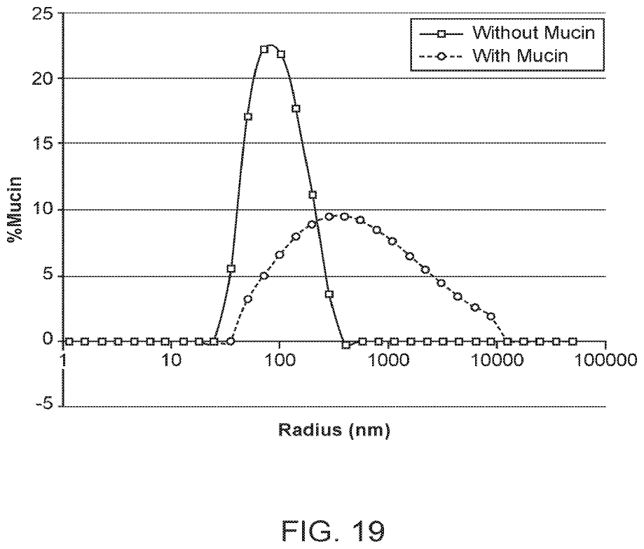

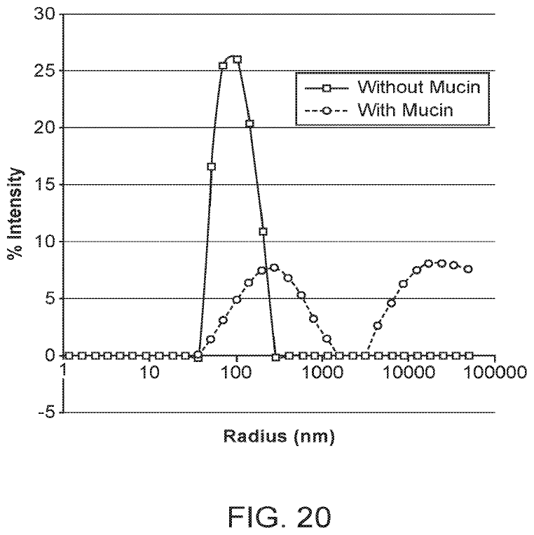

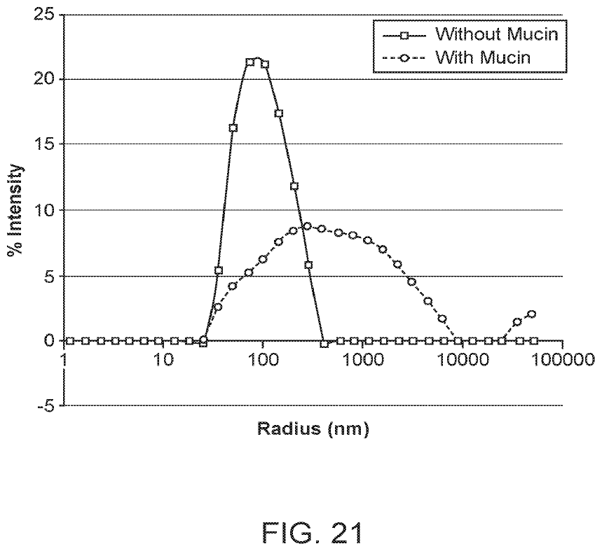

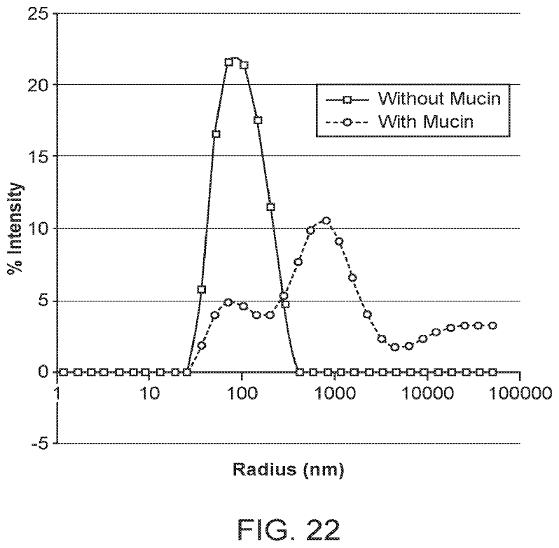

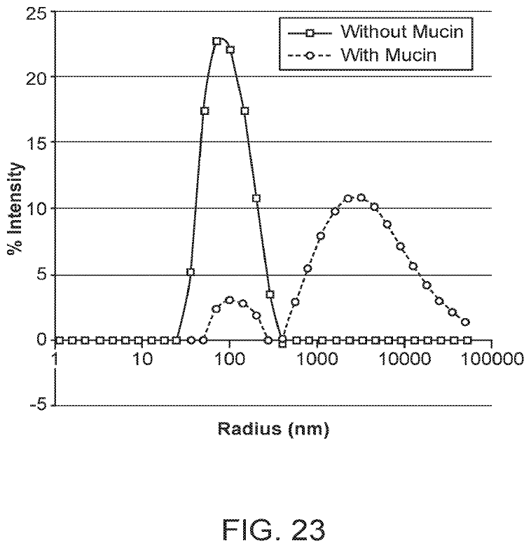

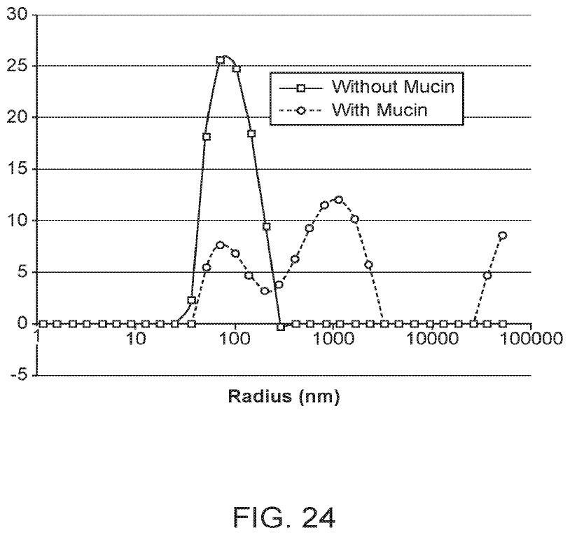

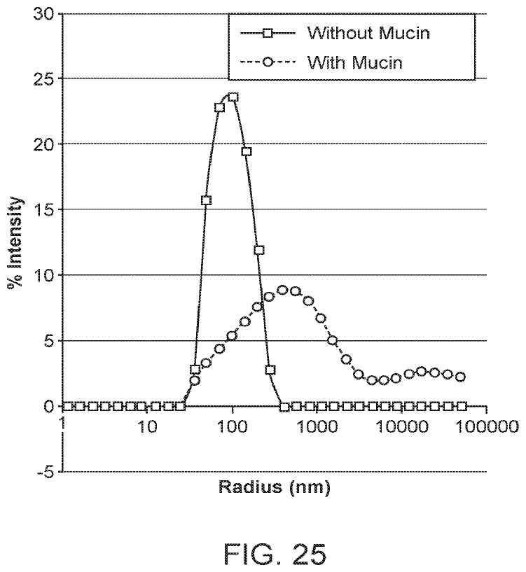

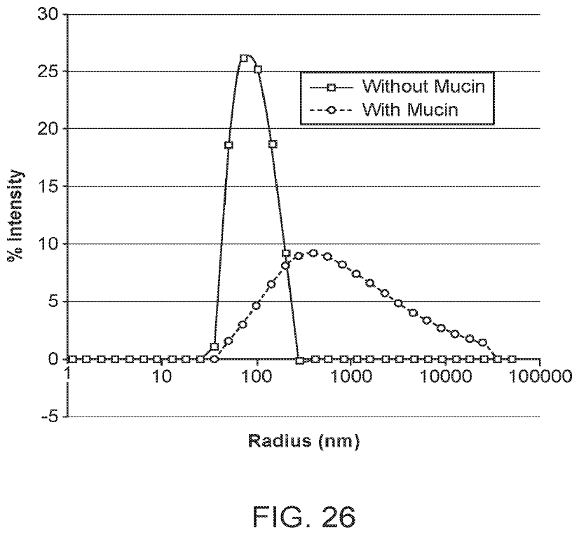

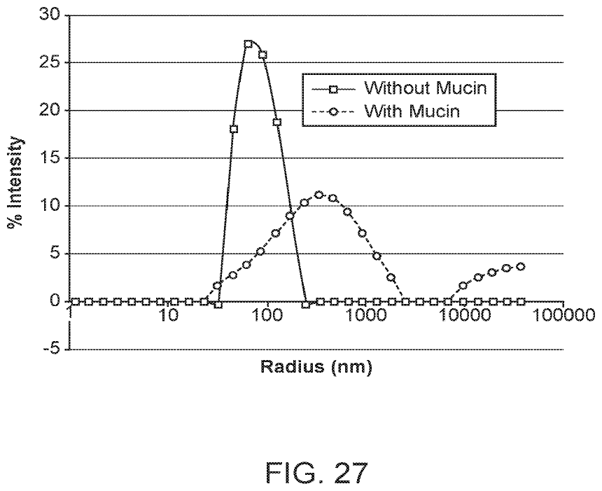

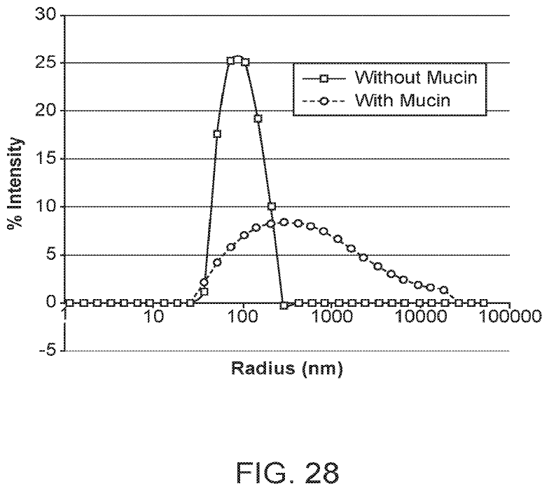

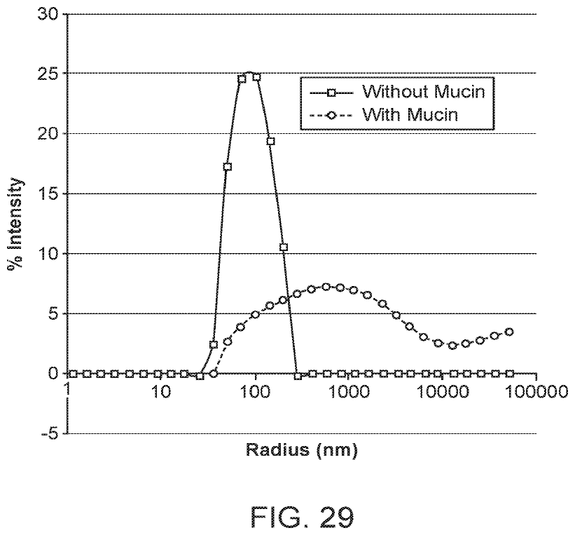

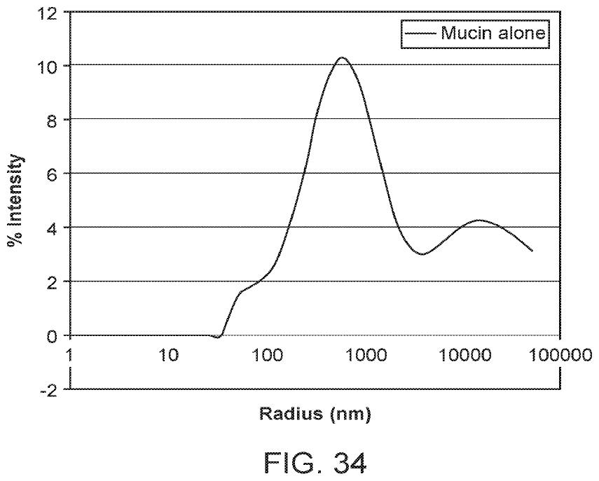

For each sample, purified 0.5 mg/mL mucin from porcine stomach (Sigma Aldrich) was added to the sample at a sample:mucin volume ratio of 5:2. Dynamic light scattering was used to measure changes in light scattering in the presence of and without mucin for each sample. A shift in light intensity peak was determined to be due to interaction with the mucin as demonstrated by the lipid base system without PEG (FIG. 2) having a shift whereas the system containing 5% mol DSPE-SS-PEG (FIG. 3) having no shift in peak in the presence of mucin. Data for each peptide tested is shown in FIGS. 1-29 and FIG. 34 shows DLS data of mucin alone. In particular, FIG. 2 shows base system (30/60/10 MVL5/DOPC/Chol) DLS Mucin Interaction study. Base system showed a shift in the intensity peak demonstrating mucin interaction. FIG. 3 shows base system containing 5% DSPE-SS-PEG. The system showed no peak shift in the presence of mucin demonstrating a lack of mucin interaction. FIG. 4 shows SEQ ID NO. 36 conjugated systems, where disappearance of the system peak was observed demonstrating mucin interaction. FIG. 5 shows SEQ ID NO. 1 conjugated systems where no peak shift was observed in the presence of mucin. Thus SEQ ID NO. 1 was found not to interact with mucin. FIG. 6 shows SEQ ID NO. 2 conjugated systems where peak shifting was observed in the presence of mucin. Thus SEQ ID NO. 2 was found to interact with mucin. FIG. 7 shows SEQ ID NO. 3 conjugated systems where peak shifting was observed in the presence of mucin. Thus SEQ ID NO. 3 was found to be interacting with mucin. FIG. 8 shows SEQ ID NO. 4 conjugated systems, where peak shifting was not observed in the presence of mucin. Thus SEQ ID NO. 4 was found not to interact with mucin.” FIG. 9 shows SEQ ID NO. 5 conjugated systems where peak shifting was not observed in the presence of mucin. Thus SEQ ID NO. 5 was found not to interact with mucin. FIG. 10 shows SEQ ID NO. 6 conjugated systems where peak shifting was not observed in the presence of mucin. Thus SEQ ID NO. 6 was found not to interact with mucin. FIG. 11 show SEQ ID NO. 7 conjugated systems where peak shifting was not observed in the presence of mucin. Thus SEQ ID NO. 7 was found not to interact with mucin. FIG. 12 shows SEQ ID NO. 8 conjugated systems where peak shifting was observed in the presence of mucin. Thus SEQ ID NO. 8 was found to interact with mucin. FIG. 13 shows SEQ ID NO. 9 conjugated systems where a shift in peak was observed in the presence of mucin. Thus SEQ ID NO. 9 was found to interact with mucin. FIG. 14 shows SEQ ID NO. 10 conjugated systems where peak shifting was observed in the presence of mucin. Thus, SEQ ID NO. 10 was found to be interacting with mucin. FIG. 15 shows SEQ ID NO. 12 conjugated systems where peak shifting was observed in the presence of mucin. Thus SEQ ID NO. 12 was found to be interacting with mucin. FIG. 16 show SEQ ID NO. 13 conjugated systems where peak shifting was observed in the presence of mucin. Thus SEQ ID NO. 13 was found to be interacting with mucin. FIG. 17 shows SEQ ID NO. 14 conjugated systems where no peak shift was observed in the presence of mucin demonstrating a lack of interaction with mucin. FIG. 18 shows that SEQ ID NO. 15 conjugated systems have a shift in peak in the presence of mucin, and were thus found to be mucus interacting. FIG. 19 shows that SEQ ID NO. 16 conjugated systems have a shift in peak in the presence of mucin, and were thus found to be mucin interacting. FIG. 20 shows that SEQ ID NO. 17 conjugated systems were found to have their peak shifted in the presence of mucin, and were thus found to be mucin interacting. FIG. 21 shows that SEQ ID NO. 19 conjugated systems were found to have their peak shifted in the presence of mucin and were thus found to be mucin interacting. FIG. 22 shows that SEQ ID NO. 20 conjugated systems had their peak not shifted in the presence of mucin and were thus not found to be mucin interacting. FIG. 23 shows that SEQ ID NO. 21 conjugated systems had their peak not shifted in the presence of mucin thus were found to not be mucin interacting. FIG. 24 shows that SEQ ID NO. 22 conjugated systems had their peak not shifted in the presence of mucin, and thus were found to not interact with mucin. FIG. 25 shows that SEQ ID NO. 23 conjugated systems had their peak shifted in the presence of mucin, and thus were found to be mucin interacting. FIG. 26 shows SEQ ID NO. 24 conjugated systems had their peak shifted in the presence of mucin, and thus were found to be mucin interacting. FIG. 27 shows that SEQ ID NO. 26 conjugated systems had their peak shifted in the presence of mucin, and thus were found to be mucin interacting. FIG. 28 shows that SEQ ID NO. 32 conjugated systems had their peak shifted in the presence of mucin and thus were found to be mucin interacting. FIG. 29 shows that SEQ ID NO. 34 conjugated systems had their peak shifted in the presence of mucin and thus were found to be mucin interacting.

A summary of table of peptides that were found to be mucus penetrating using this assay is shown below:

| Interacting with Mucin | Not Interacting with Mucin |
|---|---|
| Base system (control) | 5% mol DSPE-SS-PEG2k system (control) |
| SEQ ID NO. 36 | SEQ ID NO. 1 |
| SEQ ID NO. 2 | SEQ ID NO. 4 |
| SEQ ID NO. 3 | SEQ ID NO. 5 |
| SEQ ID NO. 8 | SEQ ID NO. 6 |
| SEQ ID NO. 9 | SEQ ID NO. 7 |
| SEQ ID NO. 10 | SEQ ID NO. 14 |
| SEQ ID NO. 12 | SEQ ID NO. 20 |
| SEQ ID NO. 13 | SEQ ID NO. 21 |
| SEQ ID NO. 15 | SEQ ID NO. 22 |
| SEQ ID NO. 16 | |
| SEQ ID NO. 17 | |
| SEQ ID NO. 19 | |
| SEQ ID NO. 23 | |
| SEQ ID NO. 24 | |
| SEQ ID NO. 26 | |
| SEQ ID NO. 32 | |
| SEQ ID NO. 34 | |

### Example 10: Hydropathy Scores

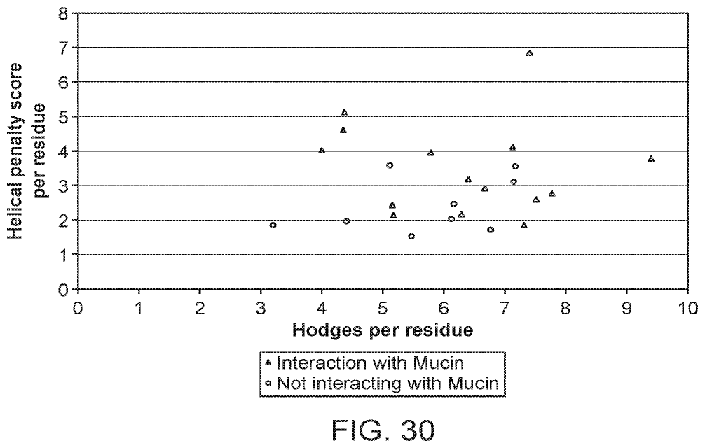

Various exemplary mucus-penetrating peptides of this disclosure were analyzed according to their hydropathy scores using the Hodges method. Furthermore, since the mucus is a hydrophobic environment, there may be a higher tendency for the peptides to form alpha helices thus hiding their residues internally. Thus, the average alpha helical penalty score per residue was also calculated using experimentally determined values from Pace and Scholtz (1998) A helix propensity scale based on experimental studies of peptides and proteins Biophys J. 1998 July; 75(1):422-7. In FIG. 30 it is shown that the peptides that did not interact with mucin (see above Table, SEQ ID Nos. 1, 4-7, 14, and 20-22) were found to have upper bounds for both hydropathy and alpha helical penalty (the higher the penalty, the less likely they are to form alpha helices). No such bounds were found for the mucus interacting peptides (see above Table, SEQ ID Nos. 36, 2-3, 8-10, 12-13, 15-17, 19, 23-24, 26, 32, and 34).

### Example 11: Ex-Vivo Studies

To encapsulate nucleic acid: Lipids were dissolved in ethanol or any organic solvent and heated above their phase transition temperature. Nucleic acid was dissolved in an aqueous buffer heated above the phase transition temperature of the lipids. The aqueous buffer pH was set at below the pKa of the bile salt and the cationic lipids. In this way, the lipids are strongly cationic when formulated with the nucleic acids. The lipids and nucleic acids were mixed using microfluidic channels. Alternatively, other forms of mixing may be used. The pH was raised to neutral and the sample was concentrated, and ethanol removed using dialysis or other methods that are known to the industry.

Protocol:

Materials: DODMA (Sigma Aldrich), DOPE (Avanti Polar Lipids), DMG-PEG 2000 (Avanti Polar Lipids), DiI (ThermoFisher Scientific),

Formulation

300 μg of plasmid DNA encoding for Gaussia Luciferase under a CMV promoter was dissolved in a final volume of 3 mL water. DODMA, DOPE, DMG-PEG2000 were mixed in ethanol according to their mole and cationic lipid:nucleic acid ratio. The cationic lipid: nucleotide molar ratio was kept constant at 12. When lipids were fluorescently labelled with DiI at 0.5% mol of the total lipid moles. Ethanol volume was raised to 1 mL. Samples were mounted into syringes on the Nanoassemblr Benchtop (Precision NanoSystems, BC). Samples were mixed using the NanoAssemblr Benchtop microfluidic chip system with a flow rate of 6 mL/min. Ethanol was removed using dialysis overnight.

The following formulations were made:

| Lipid formula | # | Mole ratios |
|---|---|---|
| DODMA/DOPE/DMG-PEG2000/DiI/ | 1 | 45/45/10/.5/ |
| SEQ ID NO. 1 | | .32 |
| DODMA/DOPE/DMG-PEG2000/DiI | 2 | 45/45/10/.5 |

*Where the formulation % mol do not equal 100, it is because of rounding errors.

Mucus Penetration

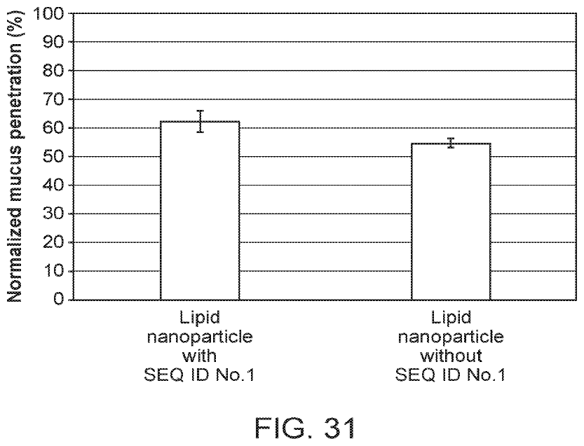

Purified mucus taken from fresh porcine intestine was mixed with PBS and applied onto the microporous membrane of a Transwell® Permeable Support (Corning Inc, NY). PBS was also added into the lower acceptor compartment. An adequate amount of DiI labeled lipid nanoparticle was applied onto the mucus and allowed to incubate. Every 30 minutes, samples were taken out of the acceptor compartment and their relative fluorescence intensity was taken by exciting at 510 nm and measuring emission at 565 nm. A non-mucus control experiment consisting of the same experiment but without mucus was conducted. After compensating for the fluorescence intensity lost in sample collection and normalizing the mucus sample fluorescence intensity with that of the non-mucus sample, the percent mucus penetration of the lipid nanoparticle was calculated. The data presented represents the mucus penetration after incubating for 90 minutes (see FIG. 31). SEQ ID NO: 1 coupled lipid nanoparticle showed similar or slightly higher mucus penetration than lipid nanoparticle without SEQ ID NO. 1 confirming the observation made from the mucin interaction assay.

### Example 12: In-Vivo Distribution Assay

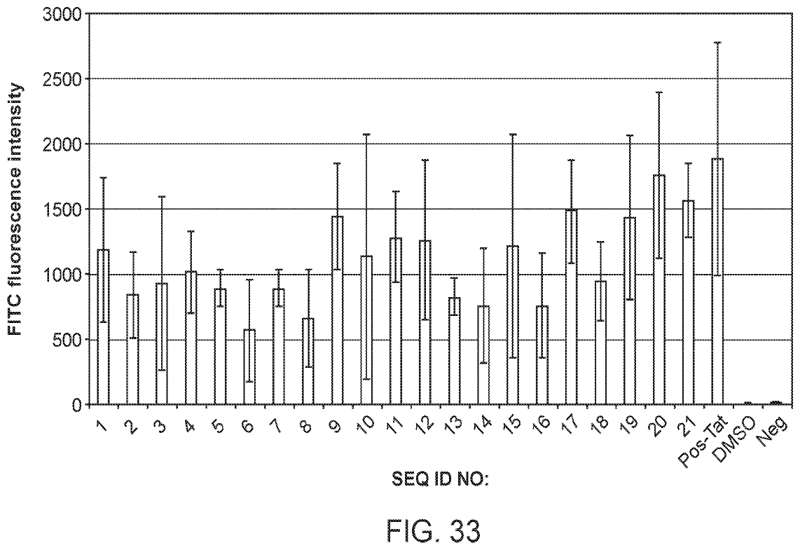

Three separate lipid nanoparticle formulations were prepared, one coupled to SEQ ID NO: 29, another coupled to transactivator of transcription peptide (TAT; SEQ ID NO:37) and the last without any coupling, all carried plasmid DNA and included 0.5% mol DiI and DiO labeling. 30 μg of DNA encapsulated in the DiI/DiO lipid nanoparticle are dosed intrarectally in BALB/c female mice (Charles River Laboratory, MA) anesthetized under 1-3% isofluorane. After 4 hours, the mice are euthanized under 25% CO2 and the colon is removed and embedded in optimal cutting temperature (OCT) media. The OCT media sections are flash frozen at −80 degrees Celsius and intestinal sections are taken in a cryostat. Epifluorescence images are taken of crypts under 531 nm excitation with 593 nm emission and overlaid with brightfield illumination. FIGS. 32A-32B. FIG. 32A shows images of lipid nanoparticle formulations without coupled peptide (top panel is a bright field image; middle panel is a dye channel image; and bottom panel is dye channel and bright field combined image). FIG. 32B shows images of lipid nanoparticle formulations with the TAT peptide (SEQ ID No. 37) (top panel is a bright field image; middle panel is a dye channel image; and bottom panel is dye channel and bright field combined image). FIG. 33C shows images of lipid nanoparticle formulations with the SEQ ID No. 29 (top panel is a bright field image; middle panel is a dye channel image; and bottom panel is dye channel and bright field combined image).

As demonstrated in the images, TAT reduced the distribution of the particle at the surface of the intestinal epithelial cells as compared to the base lipid nanoparticle, likely because it adhered to the mucus. SEQ ID NO; 29 had stronger signal and more spread out distribution at the surface of the intestinal epithelium. FIG. 33A-C

While some embodiments have been shown and described herein, such embodiments are provided by way of example only. Numerous variations, changes, and substitutions will now occur to those skilled in the art without departing from the invention. It should be understood that various alternatives to the embodiments of the invention described herein can be employed in practicing the invention.

1. A composition comprising a peptide, a cargo and a delivery vehicle, wherein the peptide is a mucus-penetrating peptide, the peptide is conjugated directly or indirectly to the delivery vehicle to form a peptide-delivery vehicle conjugate, the delivery vehicle comprises at least one mucus-penetrating feature and the delivery vehicle partially or fully encapsulates the cargo.

2. The composition of claim 1, wherein the peptide or a portion thereof is exposed on the surface of the peptide-delivery vehicle conjugate.

3. The composition of claim 1, wherein the peptide is selected from the group consisting of SEQ ID Nos. 1-35.

4. The composition of claim 1, wherein the average hydropathy of the amino acids of the peptide as measured by a Hodges score is less than or equal to 10 at pH 7.

5. The composition of claim 4, wherein the peptide comprises from 3 to 100 amino acids; and wherein the total number of amino acids with a Hodges score greater than 10 comprises no more than about 40% of the total number of amino acids in the peptide; and wherein the peptide comprises less than 5 pairs of adjacent amino acids where each amino acid of the pair has a Hodges score greater than 10.

6. The composition of claim 5, wherein the net charge of the peptide is less than about +2.

7. The composition of claim 5, wherein if the peptide comprises one or more cysteines, the one or more cysteines do not contain a free thiol.

8. The composition according to claim 1, wherein the composition is comprised within a nanoparticle.

9. The composition of claim 8, wherein the peptide is conjugated directly to the nanoparticle.

10. The composition of claim 8, wherein the nanoparticle has a diameter of no more than 500 nm.

11. (canceled)

12. (canceled)

13. The composition according to claim 9, wherein the nanoparticle comprises a lipid structure, wherein the lipid structure is selected from a liposome, a liposomal polyplex, a lipid nanoparticle and a lipoplex.

14. (canceled)

15. The composition of according to claim 1, wherein the at least one mucus-penetrating feature comprises one or more features selected from the group consisting of a surface modification to the delivery vehicle, a zwitterionic feature of the delivery vehicle, and a mucus-penetrating lipid composition of the delivery vehicle.

16. (canceled)

17. The composition of claim 15, wherein the surface modification is selected from one or more of polyethylene glycol, poly (2-alkyl-2-oxazoline), poly(2-ethyl-2-oxazoline), and poly(2-methyl-2-oxazaline), a salt thereof, a di block polymer and a tri block polymer thereof.

18. The composition according to claim 15, wherein the mucus-penetrating peptide is conjugated directly to the delivery vehicle.

19. (canceled)

20. (canceled)

21. The composition of claim 18, wherein the mucus-penetrating peptide is conjugated directly to a lipid structure or surface modification comprised by the delivery vehicle.

22. The composition according to claim 1, wherein the cargo comprises one or more of a nucleic acid, protein, a nanoparticle, a small chemical molecule, a dye, a drug, or a therapeutic molecule.

23. The composition of claim 22, wherein the nucleic acid encodes for a protein or a biologically active portion of a protein directed to treating a disease or condition.

24. The composition of claim 23, wherein the disease or condition is a disease or condition that affects the gastrointestinal tract, wherein the disease or condition is at least one of: congenital diarrhea disease, irritable bowel syndrome, chronic inflammatory bowel disease, microvillus inclusion syndrome, familial polyposis (FAP), attenuated FAP, colorectal cancer, or any combination thereof.

25-31. (canceled)

32. A method of making a composition comprising a mucus-penetrating conjugate, the method comprising:
(a) selecting a peptide with at least one cell-penetrating property and at least one mucus-penetrating property;
(b) selecting a delivery vehicle with at least one mucus-penetrating property; and
(c) conjugating, indirectly or directly, the peptide and the delivery vehicle.

33-63. (canceled)

64. A method of treating a disease or condition characterized by having at least one tissue targeted for therapy wherein the tissue comprises a layer of mucus, the method comprising administering a composition according to claim 1.

65-67. (canceled)


SEQUENCE LISTING (55 sequences)

SEQ ID NO: 1 - protein, 10 aa, Unknown
  Description of Unknown Mucus-Penetrating peptide
MATKGGTVKA

SEQ ID NO: 2 - protein, 10 aa, Unknown
  Description of Unknown Mucus-Penetrating peptide
MAKPAQGAKY

SEQ ID NO: 3 - protein, 10 aa, Unknown
  Description of Unknown Mucus-Penetrating peptide
MSVTGGKMAP

SEQ ID NO: 4 - protein, 12 aa, Unknown
  Description of Unknown Mucus-Penetrating peptide
TPKTMTQTYDFS

SEQ ID NO: 5 - protein, 12 aa, Unknown
  Description of Unknown Mucus-Penetrating peptide
NSGTMQSASRAT

SEQ ID NO: 6 - protein, 12 aa, Unknown
  Description of Unknown Mucus-Penetrating peptide
QAASRVENYMHR

SEQ ID NO: 7 - protein, 12 aa, Unknown
  Description of Unknown Mucus-Penetrating peptide
KTIEAHPPYYAS

SEQ ID NO: 8 - protein, 12 aa, Unknown
  Description of Unknown Mucus-Penetrating peptide
EPDNWSLDFPRR

SEQ ID NO: 9 - protein, 12 aa, Unknown
  Description of Unknown Mucus-Penetrating peptide
NYTTYKSHFQDR

SEQ ID NO: 10 - protein, 12 aa, Unknown
  Description of Unknown Mucus-Penetrating peptide
YPYDANHTRSPT

SEQ ID NO: 11 - protein, 12 aa, Unknown
  Description of Unknown Mucus-Penetrating peptide
DPATNPGPHFPR

SEQ ID NO: 12 - protein, 12 aa, Unknown
  Description of Unknown Mucus-Penetrating peptide
HPGSPFPPEHRP

SEQ ID NO: 13 - protein, 12 aa, Unknown
  Description of Unknown Mucus-Penetrating peptide
TSHTDAPPARSP

SEQ ID NO: 14 - protein, 7 aa, Unknown
  Description of Unknown Mucus-Penetrating peptide
RQSAGVL

SEQ ID NO: 15 - protein, 23 aa, Unknown
  Description of Unknown Mucus-Penetrating peptide
STSTVSTPVPPPVDDTTWLQSAS

SEQ ID NO: 16 - protein, 21 aa, Unknown
  Description of Unknown Mucus-Penetrating peptide
RQSAGVLGFAPTNIDDTSFHA

SEQ ID NO: 17 - protein, 8 aa, Unknown
  Description of Unknown Mucus-Penetrating peptide
RQWVGDRA

SEQ ID NO: 18 - protein, 10 aa, Unknown
  Description of Unknown Mucus-Penetrating peptide
RQSVLDSWGG

SEQ ID NO: 19 - protein, 11 aa, Unknown
  Description of Unknown Mucus-Penetrating peptide
RWQVGDRADGE

SEQ ID NO: 20 - protein, 23 aa, Unknown
  Description of Unknown Mucus-Penetrating peptide
VGDDSGGFSTTVSTEQNVPDPQV

SEQ ID NO: 21 - protein, 14 aa, Unknown
  Description of Unknown Mucus-Penetrating peptide
ADDLENVNEGMRIH

SEQ ID NO: 22 - protein, 28 aa, Unknown
  Description of Unknown Mucus-Penetrating peptide
LSTAADMQGVVTDGMASGLDKDYLKPDD

SEQ ID NO: 23 - protein, 12 aa, Unknown
  Description of Unknown Mucus-Penetrating peptide
PSSSSSSRIGDP

SEQ ID NO: 24 - protein, 15 aa, Unknown
  Description of Unknown Mucus-Penetrating peptide
DPVDTPNPTRRKPGK

SEQ ID NO: 25 - protein, 8 aa, Unknown
  Description of Unknown Mucus-Penetrating peptide
TYRFRGPD

SEQ ID NO: 26 - protein, 12 aa, Unknown
  Description of Unknown Mucus-Penetrating peptide
DATDRFHGPDAL

SEQ ID NO: 27 - protein, 26 aa, Unknown
  Description of Unknown Mucus-Penetrating peptide
DPKGDPKGVTVTVTVTVTGKGDPKPD

SEQ ID NO: 28 - protein, 17 aa, Unknown
  Description of Unknown Mucus-Penetrating peptide
TVDNPASTTNKDKLFAV

SEQ ID NO: 29 - protein, 17 aa, Unknown
  Description of Unknown Mucus-Penetrating peptide
TVDNDAPTKRASKLFAV

SEQ ID NO: 30 - protein, 7 aa, Unknown
  Description of Unknown Mucus-Penetrating peptide
EHGAMEI

SEQ ID NO: 31 - protein, 53 aa, Unknown
  Description of Unknown Mucus-Penetrating peptide
NSDSECPLSHDGYCLHDGVCMYIEALDKYACNCVVGYIGERCQYRDLKWWELR

SEQ ID NO: 32 - protein, 12 aa, Unknown
  Description of Unknown Mucus-Penetrating peptide
NIENSTLATPLS

SEQ ID NO: 33 - protein, 12 aa, Unknown
  Description of Unknown Mucus-Penetrating peptide
NSGTMQSASRAT

SEQ ID NO: 34 - protein, 12 aa, Unknown
  Description of Unknown Mucus-Penetrating peptide
TSHTDAPPARSP

SEQ ID NO: 35 - protein, 26 aa, Unknown
  Description of Unknown Mucus-Penetrating peptide
AEKVDPVKLNLTLSAAAEALTGLGDK

SEQ ID NO: 36 - protein, 10 aa, Unknown
  Description of Unknown Mucus-Penetrating peptide
LIIYRDLISH

SEQ ID NO: 37 - protein, 12 aa, Human immunodeficiency virus
GRKKRRQRRRPQ

SEQ ID NO: 38 - protein, 10 aa, Artificial Sequence
  Description of Artificial Sequence Synthetic peptide
  MOD_RES (1)..(1): Any amino acid
XGAKGAGVGL

SEQ ID NO: 39 - protein, 10 aa, Artificial Sequence
  Description of Artificial Sequence Synthetic peptide
  MOD_RES (1)..(1): Trp, Phe, n-Leu, Leu, Ile, Met, n-Val, Tyr, Val, Pro, Cys, Ala, Glu, Thr, Asp, Gln, Ser, Asn, Gly, Arg, His, Lys, or Orn
XGAKGAGVGL

SEQ ID NO: 40 - protein, 7 aa, Simian virus 40
PKKKRKV

SEQ ID NO: 41 - protein, 16 aa, Unknown
  Description of Unknown Nucleoplasmin bipartite NLS sequence
KRPAATKKAGQAKKKK

SEQ ID NO: 42 - protein, 9 aa, Unknown
  Description of Unknown C-myc NLS sequence
PAAKRVKLD

SEQ ID NO: 43 - protein, 11 aa, Unknown
  Description of Unknown C-myc NLS sequence
RQRRNELKRSP

SEQ ID NO: 44 - protein, 38 aa, Homo sapiens
NQSSNFGPMKGGNFGGRSSGPYGGGGQYFAKPRNQGGY

SEQ ID NO: 45 - protein, 42 aa, Unknown
  Description of Unknown IBB domain from importin-alpha sequence
RMRIZFKNKGKDTAELRRRRVEVSVELRKAKKDEQILKRRNV

SEQ ID NO: 46 - protein, 8 aa, Unknown
  Description of Unknown Myoma T protein sequence
VSRKRPRP

SEQ ID NO: 47 - protein, 8 aa, Unknown
  Description of Unknown Myoma T protein sequence
PPKKARED

SEQ ID NO: 48 - protein, 8 aa, Homo sapiens
PQPKKKPL

SEQ ID NO: 49 - protein, 12 aa, Mus musculus
SALIKKKKKMAP

SEQ ID NO: 50 - protein, 5 aa, Influenza virus
DRLRR

SEQ ID NO: 51 - protein, 7 aa, Influenza virus
PKQKKRK

SEQ ID NO: 52 - protein, 10 aa, Hepatitis delta virus
RKLKKKIKKL

SEQ ID NO: 53 - protein, 10 aa, Mus musculus
REKKKFLKRR

SEQ ID NO: 54 - protein, 20 aa, Homo sapiens
KRKGDEVDGVDEVAKKKSKK

SEQ ID NO: 55 - protein, 17 aa, Homo sapiens
RKCLQAGMNLEARKTKK